(19) United States
(12) Patent Application Publication
(10) Pub. No.: US 2006/0205071 A1
(43) Pub. Date: Sep. 14, 2006

(54) Methods for ex-vivo expanding stem/progenitor cells

(75) Inventors: Arik Hasson, Kiryat-Ono (IL); Frida Grynspan, Mevasseret Zion (IL)

Correspondence Address: Martin D Moynihan;Prtsi, PO Box 16446, Arlington, VA, 22215, US

(73) Assignee: Gamida-Cell Ltd., Jerusalem (IL)

(21) Appl. No.: 10/564,777

(22) PCT Filed: Jul. 15, 2004
(86) PCT No.: PCT/IL04/00643

§ 371 Date: Jan. 17, 2006

Related U.S. Application Data
(60) Provisional application No. 60/487,623, filed on Jul. 17, 2003.
(60) Provisional application No. 60/490,268, filed on Jul. 28, 2003.
(60) Provisional application No. 60/503,884, filed on Sep. 22, 2003.

(51) Int. Cl. C12N 5/08

(52) U.S. Cl. 435/366; 435/372

(57) ABSTRACT

Methods of ex-vivo expansion of fetal and/or adult progenitor, and umbilical cord blood, bone marrow or peripheral blood derived stem cells in bioreactors for bone marrow transplantation, transfusion medicine, regenerative medicine and gene therapy.

### FIELD AND BACKGROUND OF THE INVENTION

The present invention relates to methods of ex-vivo expansion and culture of progenitor and stem cells, to expanded populations of renewable progenitor and stem cells and to their uses. In particular, fetal and/or adult progenitor, and umbilical cord blood, bone marrow or peripheral blood derived stem cells can be expanded ex-vivo in bioreactors and grown in large numbers according to the methods of the present invention. Populations of stem and progenitor cells expanded according to the methods of the present invention can be used in bone marrow transplantation, transfusion medicine, regenerative medicine and gene therapy.

### Introduction

The ex vivo expansion of stem cells of hematopoietic (HSC) and other origin, is one of the most challenging objectives currently facing the field of cellular biotechnology. This rapidly growing area of tissue engineering has many potential applications in bone marrow transplantation, transfusion medicine, regenerative medicine or gene therapy. Over the last few years much progress has been made in understanding cellular differentiation: discovery of cytokines, isolation and identification of cellular subtypes and in the development of a variety of bioreactor and supporting scaffolds concepts. This, in turn, has led to clinical trials that provide a glimpse of the benefits that promise to be obtained from the use of expanded hematopoietic cells (Hoffman et al. 1993; Wagner 1993; Andrews et al. 1994; Purdy et al. 1995; Gehling et al. 1997; Bachier et al. 1999; Chabannon et al. 1999a; McNiece et al. 1999; Nielsen 1999; McNiece et al. 2000a; McNiece and Briddell 2001; Noll et al. 2002; Wolff 2002) MSC (Knutsen et al. 1998; Vilquin et al. 2002) or EPC (Chachques et al. 2002; Menasche 2002; Murohara 2003) in cellular therapy. Moreover, as the understanding of the complexity of hematopoietic, mesenchymal or endothelial stem cell transplantations in either clinical trials or in vivo animal models deepens, it is becoming clear that the number and quality of cells transplanted per body volume/weight is crucial. Higher number of cells results with better therapeutic outcome (Bensinger et al. 1996b; Chown et al. 1996; Shizuru et al. 1996; Chabannon et al. 1999b; Barker and Wagner 2002; Jaroscak et al. 2003a). In one example, it has been found that cord blood is a rich source of cells for HSC transplantation, but the low number of HSC cells collected in each cord blood unit limits common use of cord blood to children and adolescents weighing under 40 Kg, due to the minimum requirement for at least 2×10⁷ leukocytes per Kg. for successful transplantations (Kurtzberg et al. 1996; Wagner et al. 1996; Kapelushnik et al. 1998; Shpall et al. 2000; Jaroscak et al. 2003a). On the other hand, more purified populations affect the success of transplantation (Bensinger et al. 1996a; Negrin et al. 2000; Richel et al. 2000; Laughlin et al. 2001). Thus, highly sophisticated cultivation techniques and bioreactor concepts are needed to improve survival and efficacy of cellular therapy applications. Furthermore, in order to utilize cells in clinical trials and as biopharmaceutical product, highly controlled culturing conditions are required. Bioreactors, in which cells are isolated from the external surroundings, afford a means with which to accurately control and monitor those conditions.

### Cultivation of Stem and Progenitor Cells in Bioreactors

Patterns of growth and differentiation of stem cells are controlled both by cellular microenvironmental factors (epigenetic signals and development) and genetic factors (genetic development). When cultivating cells in vitro, it is essential to carefully consider the importance of chemical and physical variables such as composition of growth media, oxygen concentration, pH levels, and osmolarity, as well as the specific design and operation of the vessel in which the culture is to be maintained. It is even of greater importance when attempting to culture cells, such as stem and progenitor cells that are at the beginning of their ontogenetic development, as these cells are very sensitive to many stimuli, such as paracrine and autocrine signals, contact signals, and levels of Oxygen, carbonate, glucose and other nutrients. Attempts at overcoming the problems inherent in scaled-up ex-vivo expansion of stem cells (which tend to undergo initiation of differentiation and lose their pluripotential character when cultured in large volumes, or at higher densities; see, for example, Brott et al, Cytometry 2003; 53A:22-27, Collins et al Biotechnol and Bioengineer, 1998; 59:534-43) have yielded only partial results to date.

### Bioreactors

A bioreactor is a generalized term that essentially covers any kind of vessel that is capable of incubating cells while providing a degree of protection for the cells' environment. A bioreactor may be a static vessel such as a flask or culture bag in which the variables (such as composition of growth media, oxygen concentration, pH levels, and osmolarity) are not fully controlled and monitored. On the other hand there are fully automated electromechanical state-of-the-art bioreactors in which all the variables are monitored and controllable. Many inter-combinations between these examples are well known to one of ordinary skill in cellular biotechnology.

Three different traditional approaches for the cultivation of isolated hematopoietic stem or progenitor cells have been described in the literature: the static, the stirred and the immobilized culture. Static cultivation takes place in very simple culture systems such well plates, tissue-culture flasks or gas-permeable culture bags (Brugger et al. 1995; Alcom et al. 1996). As the former two systems do not allow cell cultivation on a clinical scale, the latter is actually the most-often used technique for stem cell expansion (Purdy et al. 1995; McNiece et al. 1999; McNiece et al. 2000a). All these systems have the advantage of being easy to handle, single-use devices, which enable an uncomplicated cell harvest. However, with all these systems, process control modulation is effected via control of the incubator environment, and there is no provision of continuous feeding. Thus, variations in culture conditions during cultivation (e.g., oxygen tension, pH, substrate, metabolite and cytokine concentrations) are critical factors in all three methods of static cultivation.

Stirred bioreactors are commonly used in animal cell culture, offering a homogenous environment, representative sampling, better access to process control and an increased oxygen transfer. Several of stirred techniques (spinner flasks and stirred vessel bioreactors) have been successfully implemented in the cultivation of hematopoietic cells (Zandstra et al. 1994; Collins et al. 1998a; Collins et al. 1998b; Noll et al. 2002).

The immobilization of stem and progenitor cells is an attempt to reach local high cell densities and to imitate the three-dimensional structure of the tissue (such as bone marrow) without the use of stromal feeder layer. In immobilized biocatalyst reactors, the cells may be immobilized in or on a carrier, immobilized by linkage among one another to form larger particles or confined within membrane barriers. Most of the reactors can be run in a batch, fed-batch or continuous mode. Immobilized bioreactors are well known in the art, such as the conventional reactors such as Continuous Stirred Tank Reactors (CSTR) and Packed Bed Reactors (PBR) as described in standard text books such as Ullmann's Encyclopedia Of Industrial Chemistry: Fifth edition, T. Campbell, R. Pfefferkom and J. F. Rounsaville Eds, VCH Publishers 1985, Vol A4, pp 141-170; Ullmann's Encyclopedia Of Industrial Chemistry: Fifth ed., B. Elvers, S. Hawkins and G. Schulz Eds, VCH Publishers, 1992, Vol B4, pp 381-433; J. B. Butt “Reaction Kinetics And Reactor Design” Prentice-Hall, Inc., 1980, pp 185-241.

A number of porous microcarriers with and without additional coating of components of an extra-cellular matrix hydrogel (e.g., collagen, fibronectin, laminin) have been investigated for use in immobilized bioreactors. Bagley et al. compared different porous materials and described a greater than sixfold expansion of colony forming cells in a long-term cultivation of CD34+ cells in tantalum-coated porous carriers, even without adding exogenous cytokines (Bagley et al. 1999). However, stem cell immobilization, especially on porous materials, requires the delicate and time-consuming detachment of the cells from the matrix prior to transplantation, a significant disadvantage compared to suspension culture.

Hollow fiber modules and the micro-encapsulation of progenitor cells have been used in hematopoietic culture, albeit with less success (Sardonini and Wu 1993). Furthermore, these approaches are not usually suitable for the clinical requirements, as the harvest of the cells is almost always impossible.

The most ambitious technique for stem cell expansion to date is the Aastrom-Replicell system (Aastrom Biosciences Inc., Ann Arbor, Mich., USA), which is an automated clinical system for the onsite expansion of stem cells in cancer therapy.

It consists of a grooved perfusion chamber for the retention of the hematopoietic cells, with the medium flow perpendicular to the channel grooves resulting in a continuous supply of fresh nutrients while metabolites are simultaneously removed (Sandstrom et al. 1995; Koller et al. 1998). This technique has already been used in a number of clinical studies (Chabannon et al. 1999a; Chabannon et al. 1999b). No incompatibility of the expanded cells was found, but the expansion of the early progenitor cells was rather inefficient (Chabannon et al. 1999a; Jaroscak et al. 2003a).

Local high cell densities, as they are realized in the pores of microcarriers or in the grooves of the Aastrom Replicell, have been considered crucial to making bone marrow MNC essentially stroma-independent, under conditions of long term cell maintenance and expansion (Koller et al. 1998). This might also be an important underlying factor contributing to the more efficient expansion of progenitors in the culture bags, where the cells accumulate in the wrinkles of the bag and reach local high cell densities (Purdy et al. 1995; McNiece et al. 1999; McNiece et al. 2000a).

Thus, bioreactors can be grouped according to general categories including: static bioreactors, stirred flask bioreactors, rotating wall vessel bioreactors, hollow fiber bioreactors and direct perfusion bioreactors. Within the bioreactors, the cells can be free, or immobilized, seeded on porous 3-dimensional scaffolds (hydrogel).

Rao et al. (US Patent Application No. 2003002363) disclosed a bioreactor for growth of hematopoietic stem cells cultivated in an inert, bio-compatible scaffold, using conventional stem cell culture medium. Using a static bioreactor having a small (100 cc) volume, they reported up to an 8.5 fold increase in CD34+ cells from cord blood after 7 days culture in the bioreactor. However, no provision for large volume medium or gas exchange was described, and thus scaling up to clinically useful volumes is not feasible due to the static nature of the bioreactor.

### Bioreactor Materials

Sensitivity to constructing material is unrelated to whether cells are anchorage-dependent or not, with material upkeep (sterilization, cleaning, and multiple using) significantly affecting culture survival (LaIuppa et al. 1997). This indicates that rather than in addition to cell-surface interactions, bioreactor materials may affect the culture by percolating toxins or binding essential media factors. This was demonstrated by the discovery that a small silicon seal inside the agitator shaft of a spinner flask may impair the ability of the culture to grow in suspension (Sardonini and Wu 1993; Zandstra et al. 1994).

### Growth Media

Cytokines are critical to all processes of hematopoiesis, such as proliferation, differentiation, adhesion and functionalities of the cells, while, in the absence of cytokines, HSC probably undergo programmed cell death, apoptosis (Cotter et al. 1994). The effects of hematopoietic cytokines are very complex and show both synergistic as well as antagonistic interactions. In the bone marrow, cytokines are produced predominantly from stromal cells (Linenberger et al. 1995; Lisovsky et al. 1996; Guerriero et al. 1997), although accessory and hematopoietic cells themselves have also been shown to secrete growth factors (such as Stem Cell Factor SCF, Linenberger et al. 1995).

Changes in the cytokine concentrations during cultivation can cause significant changes in the dynamics of proliferation and the differentiation of the cultivated cells. Therefore, the control of cytokine composition is an extremely important element of the bioprocess strategy. For the expansion of stem and progenitor cells, interleukin 6 (IL-6), stem cell factor (SCF), thrombopoietin (TPO) and flt3 ligand (FLt3) are thought to be of major significance and are mostly used in the expansion of hematopoietic stem and progenitor cells (Piciabello et al. 1997; Murray et al. 1999; Ramsfjell et al. 1999).

The number of cytokines known to influence hematopoiesis is steadily increasing but there are still growth factors in the stromal environment to be identified. This is borne out by the additional growth-supportive effects of stroma-conditioned medium on the proliferation of hematopoietic stem cells.

The choice of culture medium, especially the need to use serum, directly influences the differentiation of the cells and therefore the aims of cultivating HSC, MSC or EPC should be considered when determining the medium to be used (McAdams et al. 1996a). For example, serum normally contains TGF-b, which is known to inhibit the erythroid and megakaryocytic lineage, therefore promoting the granulocytic and macrophage differentiation (Dybedal and Jacobsen 1995). In stroma-containing culture, serum strengthens the adhesion of the cells and stabilizes the feeder layer. A further aspect which has to be considered in the use of animal serum (e.g., fetal bovine or horse) is clinical applicability, as the use of media containing components from animal sera requires significantly greater regulatory scrutiny than serum-free compositions (Sandstrom et al. 1996).

Because hematopoiesis in the bone marrow takes place under static conditions (McAdams et al. 1996a), with a continuous feed of nutrients and a simultaneous removal of waste products, several feeding strategies have been developed in the cultivation of hematopoietic cells.

Various methods have been developed for feeding cultures, ranging from feeding of cells cultured in culture bags once weekly or even less (McNiece et al. 2000b; McNiece and Briddell 2001; McNiece 2001) to half-medium exchange per week (in one feeding paradigm), and further to complete daily medium exchange in another scheme (Schwartz et al. 1991). Although continual feeding of fresh medium is theoretically beneficial for removal of waste products that may be growth-inhibitory (e.g., lactate, Patel et al. 2000), it could potentially eliminate key autocrine signals that may be important for self-regulating expansion signals emitted by the stem cell population, as well as prove costly if feeding relies on a continual supply of a costly additive.

### Expansion of Stem and Progenitor Cell Populations

While many methods for stimulating proliferation of stem and progenitor cell populations have been disclosed [see, for example, Czyz et al, Biol Chem 2003; 384:1391-409; Kraus et al., (U.S. Pat. No. 6,338,942, issued Jan. 15, 2002); Rodgers et al. (U.S. Pat. No. 6,335,195 issued Jan. 1, 2002); Emerson et al. (Emerson et al., U.S. Pat. No. 6,326,198, issued Dec. 4, 2001) and Hu et al. (WO 00/73421 published Dec. 7, 2000) and Hariri et al (US Patent Application No. 20030235909)] few provide for reliable, long-term expansion, without the accompanying differentiation that naturally occurs with growth of stem or progenitor cells in culture.

### Hematopoietic Cellular Differentiation

Much of the knowledge regarding the pathways and mechanisms underlying cellular differentiation has been extracted by careful studies on the hematopoietic system. Hematopoietic stem cells (HSCs) are responsible for maintaining normal production of blood cells (hematopoiesis), in the face of continuous cell loss to programmed cell death (apoptosis) and removal of aging cells by the reticulo-endothelial system. In the event of stress such as trauma, proper hematopoietic functioning allows release of cellular reservoirs from the marrow, downregulation of apoptosis and loss of mature cells, and enhanced proliferation of HSCs and progenitors. Such modulation of the hematopoietic system is achieved through the concerted actions of cytokines (which facilitate cell-cell and cell-matrix interactions), chemokines, and extracellular matrix (ECM) components. A single HSC can give rise to all types of hematopoietic cells, and is found in very low numbers predominantly in the bone marrow (although HSCs are also found in umbilical cord blood (UBC) and other tissues). Studies characterize human HSCs as small quiescent cells that express high levels of the surface glycoprotein CD34 (CD34+), and low or undetected levels of markers such as CD33, CD38, thy-1, and CD71, which designate a more mature progenitor population. CD34+CD38− cells (which represent <10% of the limited CD34+ cell population) can give rise to both lymphoid and myeloid cells in vitro, repopulate immune-compromised mice to high degrees, and appear critical to hematopoietic recovery of patients receiving autologous blood cell transplantation¹. In line with their ascribed role, noticeable levels of telomerase, an enzyme essential for genomic integrity and cellular proliferation, can be found in CD34+CD38− cells. Despite heightened interest in the use of these cells as therapeutic agents, population scarcity as well as poor ex vivo expansion abilities hindered their use in a clinical setting. Currently used methods of ex vivo expansion are growth of mononuclear cells, with or without prior selection for CD34 expression, with a combination of early and late growth factors; with or without serum, with or without a stromal cell layer, in stationary or rapid medium exchanged cultures, or utilizing bioreactors. In all the abovementioned systems, significant accumulation of intermediate and late progenitors is achieved, with little if any expansion of the CD34+CD38− subpopulation. Such failure in expansion of the early hematopoietic fraction is detrimental for any prospect of utilizing these expanded cultures in transplantation experiments. Current efforts are targeted at developing expansion techniques that un-couple proliferation from differentiation; such techniques may support the expansion of CD34+CD38− cells for a prolonged period without the concomitant progression of the differentiation program.

Up until recently, expansion of renewable stem cells has been achieved either by growing the stem cells over a feeder layer of fibroblast cells, or by growing the cells in the presence of the early acting cytokines thrombopoietin (TPO), interleukin-6 (IL-6), an FLT-3 ligand and stem cell factor (SCF) (Madlambayan G J et al. (2001) J Hematother Stem Cell Res 10: 481, Punzel M et al. (1999) Leukemia 13: 92, and Lange W et al. (1996) Leukemia 10: 943). While expanding stem cells over a feeder layer results in vast, substantially endless cell expansion, expanding stem cells without a feeder layer, in the presence of the early acting cytokines listed above, results in an elevated degree of differentiation (see Leslie N R et al. (Blood (1998) 92: 4798), Petzer A L et al. (1996) J Exp Med Jun 183: 2551, Kawa Y et al. (2000) Pigment Cell Res 8: 73). However, feeder-layer culture methods are poorly adaptable to large-scale expansion of stem cells, and unsuitable for growth in high volume bioreactors.

Recently, however, methods for feeder-layer free expansion of stem cells ex-vivo have been disclosed. PCT IL99/00444 to Peled et al., filed Aug. 17, 1999, which is incorporated by reference as if fully set forth by reference herein, and from which the present invention derives priority, disclosed methods of imposing proliferation yet restricting differentiation of stem and progenitor cells by treating the cells with chelators of transitional metals. While reducing the invention to practice, they uncovered that heavy metal chelators having a high affinity for copper, such as tetraethylpentamine (TEPA), greatly enhanced the fraction of CD34⁺ cell and their long-term clonability in cord-blood-derived, bone marrow-derived, and peripheral blood derived stem and progenitor cells, grown without a feeder layer. Facilitation of proliferation while inhibiting differentiation was also observed in erythroid progenitor cells, cultured mouse erythroleukemia cells, embryonal stem cells, and hepatocytes in primary hepatocyte culture treated with TEPA.

PCT IL03/00062, also to Peled et al., filed Jan. 23, 2003, which is incorporated by reference as if fully set forth herein, and from which the present invention derives priority, discloses a similar effective promotion of long term ex vivo stem cell proliferation, while inhibiting differentiation, using TEPA-Cu chelates as well as the chelator TEPA. Surprisingly, this effect of TEPA and TEPA-chelates was also demonstrated using as a starting population an un-selected peripheral mononuclear fraction. The results described there-in clearly show that stem and progenitor hematopoietic cells may be substantially expanded ex vivo, continuously over at least 12 weeks period, in a culture of mixed (mononuclear fraction) blood cells, with no prior purification of CD₃₄⁺ cells.

PCT IL 03/00064, also to Peled et al., filed Jan. 26, 2003, which is incorporated by reference as if fully set forth herein, and from which the present invention derives priority, teaches the ex-vivo expansion and inhibition of hematopoietic stem and progenitor cells using conditions and various molecules that interfere with CD38 expression and/or activity and/or with intracellular copper content, for inducing the ex-vivo expansion of hematopoietic stem cell populations. The small molecules and methods include linear polyamine chelators and their chelates, nicotinamide, a nicotinamide analog, a nicotinamide or a nicotinamide analog derivative or a nicotinamide or a nicotinamide analog metabolite, a PI 3-kinase inhibitor, conditions for reducing a capacity of the hematopoietic mononuclear cells in responding to retinoic acid, retinoids and/or Vitamin D and reducing the capacity of the cell in responding to signaling pathways involving PI 3-kinase.

Surprisingly, the inventors also showed that exposure of hepatocytes in primary culture to the small molecules, and conditions described hereinabove stimulated hepatocyte proliferation, greatly expanding the fraction of undifferentiated and immature hepatocytes (as determined by α-feto-protein expression, OC3 marker expression and oval cell morphology).

PCT IL 03/00681, also to Peled et al, filed Aug. 17, 2003, which is incorporated by reference as if fully set forth herein, and from which the present invention derives priority, discloses methods of ex-vivo expanding a population of hematopoietic stem cells present, even as a minor fraction, in hematopoietic mononuclear cells, without first enriching the stem cells, while at the same time, substantially inhibiting differentiation of the hematopoietic stem cells. Cells thus expanded can be used to efficiently provide ex-vivo expanded populations of hematopoietic stem cells without prior enrichment of the hematopoietic mononuclear cells for stem cells suitable for hematopoietic cell transplantation, for genetic manipulations for cellular gene therapy, as well as in additional application such as, but not limited to, adoptive immunotherapy, implantation of stem cells in an in vivo cis-differentiation and trans-differentiation settings, as well as, ex-vivo tissue engineering in cis-differentiation and trans-differentiation settings.

PCT IL 2004/000215, also to Peled et al., filed Mar. 4, 2004, which is incorporated by reference as if fully set forth herein, and from which the present invention derives priority, further demonstrated the self-renewal of stem/early progenitor cells, resulting in expansion and inhibition of differentiation in stem cells of hematopoietic origin and non-hematopoietic origin by exposure to low molecular weight inhibitors of PI 3-kinase, disruption of the cells' PI 3-K signaling pathways.

Israeli Patent Application No. 161903, filed May 10, 2004, also to Peled et al., which is incorporated by reference as if fully set forth herein, and from which the present invention derives priority, discloses the expansion of endodermal- and non-endodermally derived progenitor and stem cells for transplantation and the repopulation of endodermal organs.

Thus, methods are available for expansion and inhibition of differentiation of stem and progenitor cells, yielding populations of undifferentiated cells characterized by self-renewal, suitable for hematopoietic and other stem cell transplantation, for genetic manipulations for cellular gene therapy, adoptive immunotherapy, in vivo and ex-vivo cis-differentiation and trans-differentiation, organ repopulation, etc. However, there is a clear need for improved methods of growing larger quantities of stem and progenitor cells for the abovementioned clinical applications. Since large scale ex-vivo cell growth not only requires the development of new models in place of the traditional monolayer or micromass cell culture models, but also poses new technical challenges owing to the physicochemical requirements of large cell masses, it would be highly advantageous to have new methods combining the abovementioned methods with technologies for large-scale production in bioreactors.

### SUMMARY OF THE INVENTION

The present invention discloses methods of large-scale ex-vivo expansion and culture of progenitor and stem cells, expanded populations of renewable progenitor and stem cells and to their uses. In particular, fetal and/or adult progenitor, and umbilical cord blood, bone marrow or peripheral blood derived stem cells can be expanded ex-vivo and grown in large numbers according to the methods of the present invention, for example, in bioreactors. The novel methods disclosed herein may be used for scaling up of ex-vivo expansion of stem and progenitor cells, resulting in renewable populations of large numbers of stem and/or progenitor cells which can be used in bone marrow transplantation, transfusion medicine, organ repopulation, regenerative medicine and gene therapy.

While reducing the present invention to practice, it was unexpectedly found that ex-vivo expansion of stem and progenitor cells in bioreactors, using a unique culturing system, significantly improved the yield and fold increase of self-renewable stem and progenitor cells in both long and short term cultures, without the need for a feeder layer or stromal cells. Thus, it is expected that bioreactor-based ex-vivo expansion of renewable stem and progenitor cells can be used for therapeutic and clinical applications as is further detailed hereinunder.

According to one aspect of the present invention there is provided a method of ex-vivo expanding stem and/or progenitor cells, while at the same time, substantially inhibiting differentiation of the stem and/or progenitor cells, the method effected by: (a) obtaining a population of cells comprising stem and/or progenitor cells; (b) seeding the stem and/or progenitor cells into a bioreactor, and (c) culturing the stem and/or progenitor cells ex-vivo in the bioreactor under conditions allowing for cell proliferation and, at the same time, culturing the cells under conditions selected from the group consisting of: (i) conditions reducing expression and/or activity of CD38 in the cells; (ii) conditions reducing capacity of the cells in responding to signaling pathways involving CD38 in the cells; (iii) conditions reducing capacity of the cells in responding to retinoic acid, retinoids and/or Vitamin D in the cells; (iv) conditions reducing capacity of the cells in responding to signaling pathways involving the retinoic acid receptor, the retinoid X receptor and/or the Vitamin D receptor in the cells; (v) conditions reducing capacity of the cells in responding to signaling pathways involving PI 3-kinase; (vi) conditions wherein the cells are cultured in the presence of nicotinamide, a nicotinamide analog, a nicotinamide or a nicotinamide analog derivative or a nicotinamide or a nicotinamide analog metabolite; (vii) conditions wherein the cells are cultured in the presence of a copper chelator; (viii) conditions wherein the cells are cultured in the presence of a copper chelate; (ix) conditions wherein the cells are cultured in the presence of a PI 3-kinase inhibitor; thereby expanding the stem and/or progenitor cells while at the same time, substantially inhibiting differentiation of the stem and/or progenitor cells ex-vivo.

According to a further aspect of the present invention there is provided a method of transplanting ex-vivo expanded stem and/or progenitor cells into a recipient, the method effected by: (a) obtaining a population of cells comprising stem and/or progenitor cells; (b) seeding the stem and/or progenitor cells into a bioreactor; (c) culturing the stem and/or progenitor cells ex-vivo in the bioreactor under conditions allowing for cell proliferation and, at the same time, culturing the cells under conditions selected from the group consisting of: (i) conditions reducing expression and/or activity of CD38 in the cells; (ii) conditions reducing capacity of the cells in responding to signaling pathways involving CD38 in the cells; (iii) conditions reducing capacity of the cells in responding to retinoic acid, retinoids and/or Vitamin D in the cells; (iv) conditions reducing capacity of the cells in responding to signaling pathways involving the retinoic acid receptor, the retinoid X receptor and/or the Vitamin D receptor in the cells; (v) conditions reducing capacity of the cells in responding to signaling pathways involving PI 3-kinase; (vi) conditions wherein the cells are cultured in the presence of nicotinamide, a nicotinamide analog, a nicotinamide or a nicotinamide analog derivative or a nicotinamide or a nicotinamide analog metabolite; (vii) conditions wherein the cells are cultured in the presence of a copper chelator; (viii) conditions wherein the cells are cultured in the presence of a copper chelate; (ix) conditions wherein the cells are cultured in the presence of a PI 3-kinase inhibitor; (d) recovering the expanded stem and/or progenitor cells from the bioreactor, and (e) transplanting into the recipient the ex-vivo expanded stem and/or progenitor cells produced in steps (b)-(d).

According to further features in preferred embodiments of the invention described below the stem and/or progenitor cells are derived from a source selected from the group consisting of hematopoietic cells, umbilical cord blood cells, G-CSF mobilized peripheral blood cells, bone marrow cells, hepatic cells, pancreatic cells, intestinal cells, neural cells, oligodendrocyte cells, skin cells, keratinocytes, muscle cells, bone cells, chondrocytes and stromal cells.

According to further features in preferred embodiments of the invention described below the method further comprising the step of selecting a population of stem cells enriched for hematopoietic stem cells.

According to still further features in preferred embodiments of the invention described below the selection is affected via CD34.

According to further features in preferred embodiments of the invention described below the method further comprising the step of selecting a population of stem cells enriched for early hematopoietic stem/progenitor cells.

According to yet further features in preferred embodiments of the invention described below the selection is affected via CD133.

According to still further features in preferred embodiments of the invention described below step (c) is followed by a step comprising selection of stem and/or progenitor cells.

According to yet further features in preferred embodiments of the invention described below the selection is affected via CD 133 or CD 34.

According to yet further features in preferred embodiments of the invention described below the providing the conditions for cell proliferation is effected by providing the cells with nutrients and cytokines.

According to still further features in preferred embodiments of the invention described below the cytokines are selected from the group consisting of early acting cytokines and late acting cytokines.

According to further features in preferred embodiments of the invention described below the early acting cytokines are selected from the group consisting of stem cell factor, FLT3 ligand, interleukin-6, thrombopoietin and interleukin-3.

According to still further features in preferred embodiments of the invention described below the late acting cytokines are selected from the group consisting of granulocyte colony stimulating factor, granulocyte/macrophage colony stimulating factor and erythropoietin.

According to further features in preferred embodiments of the invention described below the late acting cytokine is granulocyte colony stimulating factor.

According to still further features in preferred embodiments of the invention described below the stem and/or progenitor cells are genetically modified cells.

According to yet further features in preferred embodiments of the invention described below the inhibitors of PI 3-kinase are wortmannin and/or LY294002.

According to still further features in preferred embodiments of the invention described below the bioreactor is selected from the group consisting of a static bioreactor, a stirred flask bioreactor, a rotating wall vessel bioreactor, a hollow fiber bioreactor and a direct perfusion bioreactor.

According to further features in preferred embodiments of the invention described below the static bioreactor is selected from the group consisting of well plates, tissue-culture flasks and gas-permeable culture bags.

According to yet further features in preferred embodiments of the invention described below the culturing the cells of step (c) is effected in suspension culture.

According to further features in preferred embodiments of the invention described below the culturing the cells of step (c) is effected on a porous scaffold.

According to still further features in preferred embodiments of the invention described below the porous scaffold is selected from the group consisting of poly (glycolic acid), poly (DL-lactic-co-glycolic acid), alginate, fibronectin, laminin, collagen, hyaluronic acid, Polyhydroxyalkanoate, poly 4 hydroxybutirate (P4HB) and polygluconic acid (PGA).

According to further features in preferred embodiments of the invention described below the porous scaffold comprises a hydrogel.

According to yet further features in preferred embodiments of the invention described below the seeding is static seeding or perfusion seeding.

According to still further features in preferred embodiments of the invention described below the culturing of the cells of steps (b) and (c) is effected without stromal cells or a feeder layer.

According to a still futher aspect of the present invention there is provided a conditioned medium isolated from the ex-vivo, bioreactor expanded stem and/or progenitor cell culture described hereinabove.

According to yet a further aspect of the present invention there is provided a method of preparing a stem and/or progenitor conditioned medium, and the conditioned medium prepared thereby, the method effected by: (a) establishing a stem and/or progenitor cells culture in a bioreactor as described hereinabove, thereby expanding the stem and/or progenitor cells while at the same time, substantially inhibiting differentiation of the stem and/or progenitor cells ex-vivo; and (b) when a desired stem and/or progenitor cell density has been achieved, collecting medium from the bioreactor, thereby obtaining the stem and/or progenitor cell conditioned medium.

The present invention successfully addresses the shortcomings of the presently known configurations by providing a method of propagating cells in a bioreactor, yet delaying their differentiation by interference with CD38 or PI 3-kinase expression, activity, and/or PI 3-kinase signaling.

The present invention further successfully addresses the shortcomings of the presently known configurations by enabling ex-vivo expansion of progenitor and stem cells in bioreactors, yielding large numbers of these cell populations for transplantation. Additional features and advantages of the methods of cell preparations and methods of treatment according to the present invention will become apparent to the skilled artisan by reading the following descriptions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-1C are a graphic representation of the ex-vivo expansion and inhibition of differentiation of stem cells in a static bioreactor. Hematopoietic stem/progenitor cells isolated from umbilical cord blood (UCB) mononuclear cells by magnetic activated cell sorting (MACS technology, Milteny, Bergisch-Gladbach, GmbH) were seeded in static bioreactors (gas permeable culture bags) at concentrations of 1×10⁴ cells/ml in MEM-alpha with 10% Fetal Calf Serum (FCS) containing 50 ng/ml of the following cytokines: SCF, TPO, Flt-3, IL-6, with (TEPA) or without (control) added copper chelator tetraethylenepentamine (TEPA, Aldrich, Milwaukee Wis., USA) (5 μM), and incubated for at least three weeks in a 5% CO₂ humidified incubator. FIGS. 1A and 1B show the fold expansion of indicative subpopulations of HSC at three weeks. Note the predominance of undifferentiated CD34⁺/CD38⁻ and CD34⁺/lin⁻ in cells in the TEPA expanded cultures. FIG. 1C shows the colonogenic potential of cells from Long Term Culture (LTC-CFC). Note the predominance of CFUs in the TEPA treated bioreactor cultures, also indicative of stem and early progenitor cells. CFUc frequency was calculated as number of CFUc per number of cells.

FIG. 2 is a schematic representation of the physical principles underlying the development of near-zero gravity (Σ F=0) conditions in the HARV bioreactor. Fg—gravitational force. Fc—centrifugal force. Fd—hydrodynamic drag force. ωs—settling rotation speed. Note that according to this model, the cells are in a state of free fall throughout the cultivation, making the mixing more efficient.

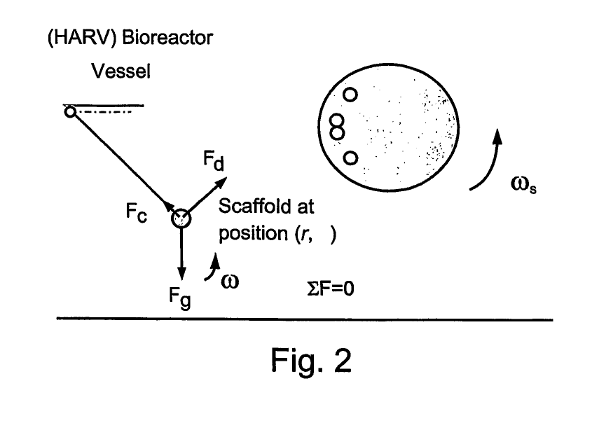

FIG. 3 is a graphic representation of the efficient expansion of hematopoietic stem cells (HSC) in large volume bioreactors. Total nucleated cells prepared on Ficoll-Hypaque gradient (1.077 g/mL; Sigma Inc, St Louis Mo., USA) from the leukocyte-rich fraction of human umbilical cord blood cultured in HSC conditions, as described hereinbelow were seeded into Teflon bags (n=19), spinner flasks (n=9), and a rotating wall vessel (HARV) bioreactor (n=9), and cultured with cytokines and TEPA, as described. Cells were seeded at 0.2-1.0×10⁴ cells/ml seeding density. Samples were analyzed for mean fold expansion at 3, 5, 7, 9, and 11 weeks. Mean fold expansion is calculated as the total number of cells (cells/ml X reactor volume) at each time point divided by the initial number of cells (seeding density X reactor volume), multiplied by the dilution factor of demi-population for feeding. Note the clear advantage of culturing in spinner flasks (>2 fold) and HARV (1.5 fold) bioreactors, most prominent at low seeding densities, compared with culturing in the static bioreactor (Teflon bags).

FIG. 4 is a graphic representation of the efficient expansion of mesechymal stem cells (MSC) in large volume bioreactors. Total nucleated cells prepared on Ficoll-Hypaque gradient (1.077 g/mL; Sigma Inc, St Louis Mo., USA) from the leukocyte-rich fraction of human umbilical cord blood cultured in MSC conditions, as described hereinbelow, were seeded into 250 ml culture flasks (n=9), spinner flasks(n=7), and a rotating wall vessel (HARV) bioreactor (n=6), and cultured with cytokines and TEPA, as described. Cells were seeded at 0.2-1.0×10⁴ cells/ml seeding density. Samples were analyzed for mean fold expansion at 3, 5, 7, 9, and 11 weeks. Mean fold expansion is calculated as the total number of cells (cells/ml X reactor volume) at each time point divided by the initial number of cells (seeding density X reactor volume), multiplied by the dilution factor of demi-population for feeding. Note the remarkable advantage of culturing mesenchymal stem cells in spinner flasks (4 fold) and HARV (>5 fold) bioreactors, most prominent at low seeding densities, compared with culturing in the static bioreactor (250 ml culture flask).

FIG. 5 is a graphic representation of the efficient expansion of endothelial stem cells (ESC) in large volume bioreactors. Total nucleated cells prepared on Ficoll-Hypaque gradient (1.077 g/mL; Sigma Inc, St Louis Mo., USA) from the leukocyte-rich fraction of human umbilical cord blood cultured in ESC conditions, as described hereinbelow were seeded into Teflon bags (n=8), spinner flasks (n=7), and a rotating wall vessel (HARV) bioreactor (n=6), and cultured with cytokines and TEPA, as described. Cells were seeded at 0.2-1.0×10⁴ cells/ml seeding density. Samples were analyzed for mean fold expansion at 3, 5, 7, 9, and 11 weeks. Mean fold expansion is calculated as the total number of cells (cells/ml X reactor volume) at each time point, divided by the initial number of cells (seeding density X reactor volume), multiplied by the dilution factor of demi-population for feeding. Note the remarkable advantage of culturing in spinner flasks (>2 fold) and HARV (>2.5 fold) bioreactors, most prominent at low seeding densities, compared with culturing in the static bioreactor (Teflon bags).

FIG. 6 is a graphic representation of the efficient expansion of the CD133+ fraction of hematopoietic stem cells (HSC) cultured in large volume bioreactors. Total nucleated cells prepared on Ficoll-Hypaque gradient (1.077 g/mL; Sigma Inc, St Louis Mo., USA) from the leukocyte-rich fraction of human umbilical cord blood cultured in HSC conditions, as described hereinbelow were seeded into Teflon bags (n=19), spinner flasks (n=9), and a rotating wall vessel (HARV) bioreactor (n=9), and cultured with cytokines and TEPA, as described. Cells were seeded at 0.2-1.0×10⁴ cells/ml seeding density. Samples were analyzed for CD133+ content, and fold expansion calculated at 3, 5 and 7 weeks. Mean CD133+ fold expansion is calculated as the total number of CD133+ cells (CD133+ cells/ml X reactor volume) at each time point divided by the initial number of cells (seeding density of CD133+ X reactor volume), multiplied by the dilution factor of demi-population for feeding. Note the clear advantage of culturing in spinner flasks (up to 1.5 fold) and HARV (up to 1.3 fold) bioreactors, most prominent at low seeding densities, compared with culturing in the static bioreactor (Teflon bags).

FIG. 7 is a graphic representation of the efficient expansion of the CD133+ fraction of hematopoietic stem cells (HSC) cultured in large volume bioreactors. Total nucleated cells prepared on Ficoll-Hypaque gradient (1.077 g/mL; Sigma Inc, St Louis Mo., USA) from the leukocyte-rich fraction of human umbilical cord blood cultured in HSC conditions, as described hereinbelow were seeded into Teflon bags (n=19), spinner flasks (n=9), and a rotating wall vessel (HARV) bioreactor (n=9), and cultured with cytokines and TEPA, as described. Cells were seeded at 0.2-1.0×10⁴ cells/ml seeding density. Samples were analyzed for CD133+ content, and mean % CD133+ calculated at 3, 5 and 7 weeks. Mean % CD133+ is calculated as the total number of CD133+ cells/ml divided by the total number of cells/ml X100 at each time point. Note the clear advantage of culturing in spinner flasks (up to 1.5 fold) and HARV (up to 1.3 fold) bioreactors, most prominent at low seeding densities, compared with culturing in the static bioreactor (Teflon bags).

FIG. 8 is a graphic representation of the efficient expansion of the CD133+/CD34− fraction of hematopoietic stem cells (HSC) cultured in large volume bioreactors. Total nucleated cells prepared on Ficoll-Hypaque gradient (1.077 g/mL; Sigma Inc, St Louis Mo., USA) from the leukocyte-rich fraction of human umbilical cord blood cultured in HSC conditions, as described hereinbelow were seeded into Teflon bags (n=19), spinner flasks (n=9), and a rotating wall vessel (HARV) bioreactor (n=9), and cultured with cytokines and TEPA, as described. Cells were seeded at 0.2-1.0×10⁴ cells/ml seeding density. Samples were analyzed for CD133 +/CD34 − content, indicating the fraction of immature, early stage stem cells in the culture. Mean % CD133 +/CD34 − was calculated at 3, 5 and 7 weeks. Mean % CD133+/CD34− is calculated as the total number of CD133+/CD34− cells/ml divided by the total number of cells/ml X100 at each time point. Note the clear advantage of culturing in spinner flasks (up to 3 fold) and HARV (up to 2.5 fold) bioreactors, most prominent at low seeding densities, compared with culturing in the static bioreactor (Teflon bags).

### DESCRIPTION OF THE PREFERRED EMBODIMENTS

The present invention discloses methods of large-scale ex-vivo expansion and culture of progenitor and stem cells, expanded populations of renewable progenitor and stem cells and to their uses. In particular, fetal and/or adult progenitor, and umbilical cord blood, bone marrow or peripheral blood derived stem cells can be expanded ex-vivo and grown in large numbers according to the methods of the present invention, for example, in bioreactors. The novel methods disclosed herein may be used for scaling up of ex-vivo expansion of stem and progenitor cells, resulting in renewable populations of large numbers of stem and/or progenitor cells. In one embodiment, the invention facilitates the efficient establishment of large scale ex-vivo expanded populations of stem and/or progenitor cells derived from cord blood, bone marrow or peripheral blood in bioreactors, suitable for bone marrow transplantation, transfusion medicine, organ repopulation, regenerative medicine and gene therapy. Additional applications may include, but are not limited to, ex-vivo trans-differentiation, ex vivo tissue engineering and ex-vivo production of endocrine hormones. The invention is particularly suited to bioreactor culture of stem and/or progenitor cells in a stromal cell free and/or feeder layer-free environment.

While reducing the present invention to practice, it was found that addition of a transition metal chelator (TEPA) to the culture medium of hematopoietic stem cells in short and long term culture in a static bioreactor dramatically increased the fraction of self-renewing, undifferentiated cells, when compared with cytokine-only cultures. Thus, the combination of bioreactor technology and improved methods of stem cell culture can provide previously unattainable numbers of clinically useful stem and/or progenitor cells, at greatly reduced cost.

The principles and operation of the present invention may be better understood with reference to the drawings and accompanying descriptions and examples.

Before explaining at least one embodiment of the invention in detail, it is to be understood that the invention is not limited in its application to the details of construction and the arrangement of the components set forth in the following description or illustrated in the Examples section. The invention is capable of other embodiments or of being practiced or carried out in various ways. Also, it is to be understood that the phraseology and terminology employed herein is for the purpose of description and should not be regarded as limiting.

Ex vivo engineering of living tissues is a rapidly developing area with the potential to impact significantly on a wide-range of biomedical applications. Major obstacles to the generation of functional tissues and their widespread clinical use are related to a limited understanding of the regulatory role of specific physicochemical culture parameters on tissue development, and the high manufacturing costs of the few commercially available engineered tissue products. By enabling reproducible and controlled changes of specific environmental factors, bioreactor systems provide both the technological means to reveal fundamental mechanisms of cell function in a 3D environment, and the potential to improve the quality of engineered tissues. In addition, by automating and standardizing tissue manufacture in controlled closed systems, bioreactors could reduce production costs, thus facilitating a wider use of engineered tissues.

While reducing the present invention to practice, it was found that stem and/or progenitor cells can be efficiently expanded ex-vivo in a bioreactor, providing a greater than 1000 fold increase in clonogenic potential of the seeded cells (CFU per 1000 cells seeded), as compared to cells receiving cytokines only, after 6-12 weeks growth. Further, it was uncovered, for the first time, that stem cells cultured in bioreactor conditions greatly exceeded the fold expansion and clonogenic potential of cells grown in other methods of culture. Thus, according to one aspect of the present invention, there is provided a method of ex-vivo expanding stem and/or progenitor cells, while at the same time, substantially inhibiting differentiation of the stem and/or progenitor cells, the method effected by: (a) obtaining a population of cells comprising stem and/or progenitor cells; (b) seeding the stem and/or progenitor cells into a bioreactor, and (c) culturing the stem and/or progenitor cells ex-vivo in the bioreactor under conditions allowing for cell proliferation and, at the same time, culturing the cells under conditions selected from the group consisting of: (i) conditions reducing expression and/or activity of CD38 in the cells; (ii) conditions reducing capacity of the cells in responding to signaling pathways involving CD38 in the cells; (iii) conditions reducing capacity of the cells in responding to retinoic acid, retinoids and/or Vitamin D in the cells; (iv) conditions reducing capacity of the cells in responding to signaling pathways involving the retinoic acid receptor, the retinoid X receptor and/or the Vitamin D receptor in the cells; (v) conditions reducing capacity of the cells in responding to signaling pathways involving PI 3-kinase; (vi) conditions wherein the cells are cultured in the presence of nicotinamide, a nicotinamide analog, a nicotinamide or a nicotinamide analog derivative or a nicotinamide or a nicotinamide analog metabolite; (vii) conditions wherein the cells are cultured in the presence of a copper chelator; (viii) conditions wherein the cells are cultured in the presence of a copper chelate; (ix) conditions wherein the cells are cultured in the presence of a PI 3-kinase inhibitor; thereby expanding the stem and/or progenitor cells while at the same time, substantially inhibiting differentiation of the stem and/or progenitor cells ex-vivo.

As used herein, the term “bioreactor” refers to any device in which biological and/or biochemical processes develop under monitored and controlled environmental and operating conditions, for example, pH, temperature, pressure, nutrient supply and waste removal. According to one embodiment of the invention, the basic classes of bioreactors suitable for use with the present invention include static bioreactors, stirred flask bioreactors, rotating wall bioreactors, hollow fiber bioreactors and direct perfusion bioreactors.

Static bioreactors differ from other types of bioreactors in the lack of provision for continuous feeding, and in the dependence on incubator environment for control of certain culture conditions. Static bioreactors commercially available include well plates, tissue culture flasks and gas-permeable culture bags. Suitable tissue culture flasks are well known in the art, for example, the CELLine dual-compartment static bioreactor (IBS, Integra Biosciences, Chur, Switzerland), which provides for separation between the medium compartment and the cell culture compartment via semi-permeable membrane. The Nunclon “Cell Factory” (Nalge-Nunc International, Naperville, Ill.) is a stackable, disposable modular tissue-culture flask which is easily seeded with cells and supplied with medium by gravity feed, prior to placement in an incubator.

The static bioreactor can be provided with low-shear mixing of gases and medium by rocker platforms within the incubators. Another suitable static bioreactor is the WAVE bioreactor system, based on the CELLBAG, from Wave Biotech LLC, (Bridgewater, N.J.). In the WAVE bioreactor, culture medium and cells only contact a presterile, disposable chamber called a Cellbag that is placed on a special rocking platform within an incubator after introduction of cells and medium, and adjustment of gases. The rocking motion of this platform induces waves in the culture fluid. These waves provide mixing and oxygen transfer, resulting in a favorable environment for cell growth that can easily support over 20×10⁶ cells/ml. The bioreactor requires no cleaning or sterilization, providing ease in operation and protection against cross-contamination.

Gas-permeable culture bags are also well known in the art. These simple single-use, disposable bioreactors are provided sterile, are sealed after filling with cells and medium, and can be incubated with or without rocking for mixing. Gas exchange is effected via a gas-permeable membrane integrated into the bag walls. Gas-permeable culture bags suitable for use in the present invention include, for example, the Optima and OrbiCell culture systems (Meta-Bios, Victoria, BC), and the LifeCell and SteriCell culture systems from Baxter (Nexell Inc, Irvine, Calif.). In one preferred embodiment, the static bioreactor is a VueLife® FEP Teflon bag (American Fluoroseal Corporation, Gaithersburg, Md.). When the VueLife® FEP Teflon bag is utilized, HSCs are incubated at 37° C. in a humidified atmosphere of 5% CO₂ in air. Isolated stem cells, from cord blood, bone marrow or other origin, are prepared and seeded into the culture bags at low initial concentration (preferably 1×10³-1×10⁵ cells/ml, more preferably 1×10⁴ cells/ml), and cultured for at least 3 weeks, with periodic replenishment of medium (“feeding”), at intervals of once per day to once a week, preferably once weekly. As described hereinbelow, stem cell proliferation and expansion is best achieved using a medium comprising a combination of nutrients and cytokines, and an effective concentration of a transition metal chelator such as TEPA. Harvest of the cells is effected by removing cells and culture medium, and optionally followed by separation and isolation of desired stem and/or progenitor cells, as described hereinbelow.

While reducing the present invention to practice, it was uncovered that culturing stem and/or progenitor cells with the transition metal chelator TEPA in a static bioreactor (VueLife® FEP Teflon bag, American Fluoroseal Corporation, Gaithersburg, Md.) produced a greatly expanded population of ex-vivo cultured stem and/or progenitor cells, having superior functional characteristics (colonogenic potential), as compared with cells cultured without TEPA. Thus, hematopoietic, mesenchymal and endothelial stem and/or progenitor cells can be efficiently expanded while inhibiting differentiation in a scaled up volume, in a static bioreactor.

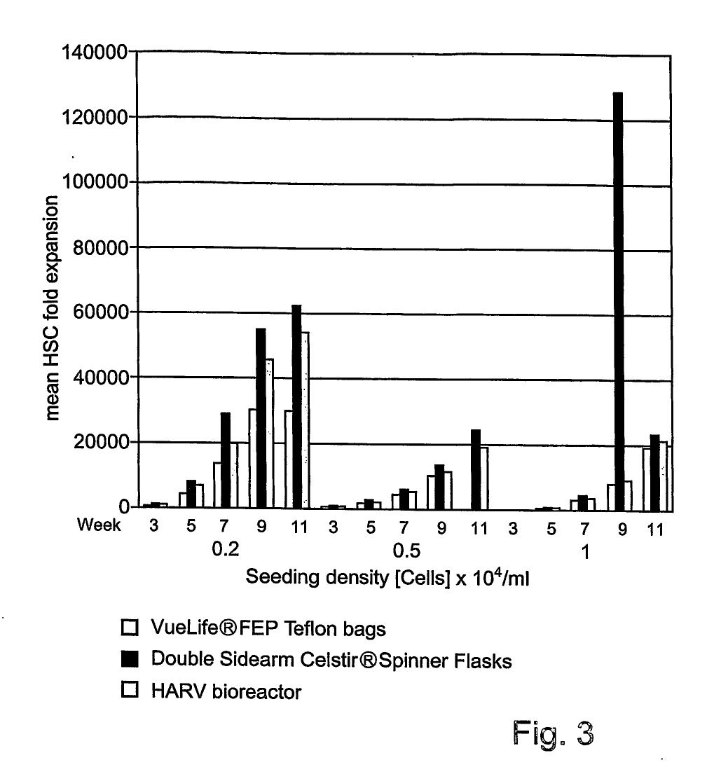

While further reducing the present invention to practice, a clear advantage, in terms of fold expansion of total hematopoietic, mesenchymal and endothelial stem cells, and specific increase in the percentage of CD133+ and CD133+/CD34− cells in culture was shown for the spinner flask and HARV bioreactors (FIGS. 3-8), as compared to cells grown in a static reactor, such as the VueLife FEB Teflon culture bag and 250 ml tissue culture flasks. While reducing the present invention to practice, it was uncovered that, in many of the bioreactor protocols tested, most significant effects on the expansion of hematopoietic, mesenchymal and endothelial stem cells, and particularly of the CD133+ fractions, was achieved at lower seeding densities. Thus, in one embodiment, the stem and/or progenitor cells are seeded in the bioreactors at cell density of about 0.05-1.5×10⁴ cell/ml. In a preferred embodiment, the cells are seeded at about 0.1-0.5×10⁴ cell/ml, and in a more preferred embodiment, about 0.2×10⁴ cell/ml.

Several types of bioreactors that portray different patterns of fluid dynamics and vessel geometry to improve mass transport are known in the art. Mechano-electrical bioreactors suitable for the cultivation of HSC, MSC or EPC or other stem cells have been described in the scientific literature (Koller et al. 1993a; Koller et al. 1993b; Zandstra et al. 1994; Koller et al. 1995; Collins et al. 1998a; Collins et al. 1998b; Kogler et al. 1998; Mantalaris et al. 1998; Chabannon et al. 1999a; Nielsen 1999; Leor et al. 2000; Banu et al. 2001; Mackin et al. 2001; Altman et al. 2002; Dar et al. 2002; Mandalam and Smith 2002; Noll et al. 2002; Sen et al. 2002a; Sen et al. 2002b; Wolff 2002; Jaroscak et al. 2003b) and have been disclosed in patents (such as in patents: US5728581, US5605822, WO9640876A1, WO02064755A2, US 5811301, WO9514078A1, WO02080995A1, WO974707, WO0046349, WO03004626A1, A2, A3, US6228635, WO0066712A3, WO0066712A2, US5985653, US5928945, US5843431, US5833979, US5824304, US5795790, US5776747, US5645043, US5635387, US5635386, WO9521911A1, US5646043, US5437994, US5605822, US5635386, US5646043, US5670351, US6326198, US5674750, US5925567, which are incorporated by reference as if fully set forth by reference herein).

Stirred flask or spinner flask bioreactors are particularly suitable for cells grown in suspension. Stirred bioreactors provide a homogeneous environment and are easy to operate, allowing sampling, monitoring and control of culture conditions. Typical operating modes include batch, fed-batch and perfusion mode (medium exchange with retention of cells by means of an external filtration module or of internal devices such as spin filters). HSCs do not require surface attachment to grow and have been successfully cultured in stirred bioreactors with improved performance, as mixing overcomes diffusion limitations of static culture systems. Stirred suspension culture systems are relatively simple and readily scalable. In addition, their relatively homogeneous nature makes them suited for the investigation of different culture parameters.

Spinner flasks are either plastic or glass bottles with a central magnetic stirrer shaft and side arms for the addition and removal of cells and medium, and gassing with CO₂ enriched air. Inoculated spinner flasks are placed on a stirrer and incubated under the culture conditions appropriate for the cell line. Cultures should be stirred at 10-250, preferably 30-100, and most preferably 50 revolutions per minute. Spinner and stirrer flask systems designed to handle culture volumes of 1-12 liters are commercially available, such as the Corning ProCulture System (Corning, Inc., Acton, Minn.), Techne Stirrer System (Techne Incorporated, Burlington, N.J.), cell culture (Bell-Flo) and bioreactor systems from Bellco Inc. (Vineland, N.J.), for example, Bellco Prod. No's. Z380482-3L capacity and Z380474-1L capacity. In a preferred embodiment, the spinner flasks are the Magna-Flex® Spinner Flasks (Wheaton Science Products, Millville, N.J.) and Double Sidearm Celstir® Spinner Flasks (Wheaton Science Products).

In one preferred embodiment, the spinner flask bioreactor (bottle) is an agitated flask constantly stirred at 50 rpm (Carrier et al. 1999). The cell constructs (or suspension) in the spinner flasks are subjected to turbulence providing not only a well-mixed environment for the cells, thus minimizing the stagnant layer at their surface, but also providing important mechanical conditioning of the stem cells. Such spinner flasks are typically equipped with probes for monitoring pH, temperatures, oxygen and CO₂ saturation, levels of metabolites such as glucose, nitrogen, amino acids, etc. in the medium, and are in fluid communication, optionally with the aid of a peristaltic pump, with fresh supplies of medium, gases, specific nutrients, and the like, and with waste removal, so that medium can be drawn off or replenished to maintain optimal conditions for stem cell expansion, at a predetermined rate.

Shear stress and turbulent eddies are sometimes a concern with stirred flask bioreactors. The dynamic laminar flow generated by a rotating fluid environment is an efficient method for reducing diffusional limitations of nutrients and wastes while minimizing levels of shear. Originally inspired by the surprising results of cell culture growth in a gravity-free environment, such as space, rotating wall vessels have been used for cell growth in-vitro with a variety of cell types (see, for example, Vunjak-Novalovic et al, J Orthop Res 1999; 17:130-38, Rhee, et al, In Vitro Cell Dev 2001; 37:127-40, Licato et al In Vitro Cell Dev, 2001; 37:121-26 and Pei, et al, FASEB J 2002; 16:1691-94). Thus, according to a further embodiment of the present invention, the bioreactor is a rotating wall vessel bioreactor. Suitable rotating wall vessel bioreactors are well known in the art, for example the HARV, Roller Cell and RCCS-1 from Synthecon (Synthecon Inc, Houston Tex.), and roller bottles of various types from Corning (Corning, Inc., Acton, Minn.). In a preferred embodiment, the RWV is a HARV from Synthecon (Synthecon Inc, Houston Tex.).

It has been shown that increase of medium exchange rates, using perfusion, leads to an extended ex vivo proliferation of human bone marrow cells. Thus, in another embodiment, the bioreactor is a perfusion chamber. Typically, “perfusion bioreactors” can be classified into two groups according to their feeding methods: while one type is fed continuously (continuous feed) the other is fed in pulses (pulse feed). Perfusion bioreactors are easily available to one of ordinary skill in the art, for example, the Corning CellCube (Corning, Inc., Acton, Minn.), and the WAVE Bioreactor with Floating Filter (WAVE Biotech, Bridgewater N.J.). U.S. Pat. No. 5,320,963 to Knaack et al., which is incorporated by reference as if fully set forth by reference herein, discloses a conical perfusion bioreactor having lamellar elements in the cell settling zone, designed for large scale culture of hematopoietic stem cells. U.S. Pat. No. 5,081,035 to Halberstadt et al, and PCT Publication WO9524464 to Bender, et al., which are incorporated by reference as if fully set forth by reference herein, disclose perfusion reactors suitable for the large scale culture of mammalian cells. Bioreactors combining the advantages of roating wall vessels and perfused culture bioreactors have been described in detail in U.S. Pat. No. 6,642,019 to Anderson, et al, and US Patent Application No. 2002146,816 to Deuser et al., which are incorporated by reference as if fully set forth by reference herein. Other such perfusion bioreactors have been described by Damen (Damen, B, 2003, available at adt.lib.swin.edu.au).

In one preferred embodiment, the perfusion bioreactor suitable for the methods of the present invention is the OPTICELL™ OPTICORE™ ceramic core S-51, S451 (flat surface area 23.8 m²), S-1251 (flat surface area 10.4 m²) or S-7 (Cellex Biosciences, Inc., Minneapolis; Minn.). Before seeding, the bioreactors are first sterilely perfused, preferably for 1-3 days, with sterile deionized water to remove any toxic substances adhering to the core. Thereafter, the core is perfused for a brief period (less than 24 hours) with sterile 25% (w/v) human serum albumin in order to coat the core with protein. The bioreactor core is then perfused for 4-24 hours with a sterile solution of an anticoagulant, preferably heparin sulfate, 100 U/mL 65 (Upjohn Co.) as a source of glycosaminoglycan and to prevent cell clumping during HSC inoculation. Following this preparation, the core is conditioned by perfusing it with sterile human HSC medium, preferably for about 12-36 hours, prior to inoculating the bioreactor with stem cells. Cell seeding, monitoring of environmental conditions, and replenishment of gas and nutrients are effected as described above for the stirred flask bioreactors.

In a further embodiment, the perfusion bioreactor is the Aastrom-Replicell system (Aastrom Biosciences Inc., Ann Arbor, Mich., USA), which is an automated clinical system for the onsite expansion of stem cells in cancer therapy.

The Aastrom-Replicell bioreactor has a grooved perfusion chamber for the retention of the hematopoietic cells, with the medium flow perpendicular to the channel grooves resulting in a continuous supply of fresh nutrients while metabolites are simultaneously removed (Sandstrom et al. 1995; Koller et al. 1998). This technique has already been used in a number of clinical studies (Chabannon et al. 1999a; Chabannon et al. 1999b). No incompatibility of the expanded cells was found, but the expansion of the early progenitor cells was rather inefficient (Chabannon et al. 1999a; Jaroscak et al. 2003a). However, none of the abovementioned studies employed the methods of HSC expansion described hereinbelow.

Local high cell densities, as they are realized in the pores of microcarriers or in the grooves of the Aastrom Replicell, have been considered crucial to making bone marrow mononuclear cells essentially stroma-independent, under conditions of long term cell maintenance and expansion (Koller et al. 1998). Without wishing to be limited to a single hypothesis, these cell densities may also be an important underlying factor contributing to the more efficient expansion of progenitors in the culture bags, where the cells accumulate in the wrinkles of the bag and reach local high cell densities (Purdy et al. 1995; McNiece et al. 1999; McNiece et al. 2000a).

Hollow fiber bioreactors can be used to enhance the mass transfer during culture of hematopoietic cells. Thus, according to a further embodiment of the present invention, the bioreactor may be a hollow fiber bioreactor. Hollow fiber bioreactors may have the stem and/or progenitor cells embedded within the lumen of the fibers, with the medium perfusing the extra-lumenal space or, alternatively, may provide gas and medium perfusion through the hollow fibers, with the cells growing within the extralumenal space. Such hollow fiber bioreactors suitable for use with the methods of the present invention have been disclosed in detail by Jauregui et al (U.S. Pat. Nos. 5,712,154 and 6,680,166) and Gloeckner et al (Biotech Prog 2001, 17:828-31), which are incorporated by reference as if fully set forth by reference herein, and are commercially available, such as the CellMax Systems supplied by Spectrum, Inc. (Rancho Dominguez, Calif.).

Additional methods of bioreactor cell culture suitable for use in the present invention include perfusion airlift bioreactors (see, for example, U.S. Pat. Nos. 5,342,781 to Su, and 4,806,484 to DeGiovanni et al, which are incorporated by reference as if fully set forth by reference herein), and packed bed bioreactors, as described in detail by Meissner et al. (Cytotechnology, 1999; 30:227-34) and Jelinek et al. (Eng Life Sci 2002; 2:15-18 and Exp Hematol 2000; 28:122-23), which are incorporated by reference as if fully set forth by reference herein. Airlift bioreactors suitable for use with the present invention are commercially available (for example, the Cytolift Glass Airlift Bioreactor, Kimble/Kontes Inc, Vineland, N.J.). In addition, growth parameters of the cell culture can be monitored in real time, and computational modeling of the growth parameters could potentially be integrated to predict the growth and development of cells in culture.

The immobilization of stem and progenitor cells is an attempt to reach local high cell densities and to imitate the three-dimensional structure of the bone marrow without the use of a stromal feeder layer. In immobilized biocatalyst reactors, the cells may be immobilized in or on a carrier, immobilized by linkage among one another to form larger particles or confined within membrane barriers.

Thus, according to one embodiment of the invention, culturing of the stem and/or progenitor cells is effected on a porous scaffold.

Much of the success of scaffolds in cell culture scale up depends on identifying an appropriate material to address the critical physical, mass transport, and biological design variables inherent to each. Hydrogels are an appealing scaffold material because they are structurally similar to the extracellular matrix of many tissues, can often be processed under relatively mild conditions, and may be delivered in a minimally invasive manner. Consequently, hydrogels have been utilized as scaffold materials for engineering tissue replacements, and expanding cell culture. The scaffold of the present invention may be made uniformly of a single polymer, co-polymer or blend thereof. However, it is also possible to form a scaffold according to the invention of a plurality of different polymers. There are no particular limitations to the number or arrangement of polymers used in forming the scaffold. Any combination which is biocompatible, may be formed into fibers, and degrades at a suitable rate, may be used. It is possible, for example, to apply polymers sequentially.

In a preferred embodiment, the biodegradable polymer is selected from the group consisting of poly (glycolic acid), poly (DL-lactic-co-glycolic acid), alginate, fibronectin, laminin, collagen, hyaluronic acid, polyhydroxyalkanoate, poly 4 hydroxybutirate (P4HB) and polygluconic acid (PGA). The fabrication and use of porous scaffolds for support of cells in scaled-up cultures is well known in the art (for a review, see Drury et al, Biomaterials 2003; 24:4337-51). U.S. Pat. No. 5,939,323 to Valentini et al, which is incorporated by reference as if fully set forth by reference herein, discloses hyaluronic acid derivatized scaffolds, and methods of forming them, for cell culture. U.S. Pat. No. 6,337,198 to Levene, which is incorporated by reference as if fully set forth by reference herein discloses the use of such biodegradable porous polymer scaffolds for cell and tissue growth. Aeschlimann et al (U.S. Pat. No. 6,630,457, which is incorporated by reference as if fully set forth by reference herein) later proposed the incorporation of functional side chain-derivatives of hyaluronic acid, and cross-linking of the scaffold polymer chains. Scaffolds can also be formed from synthetic peptide nanofiber material known as PuraMatrix, available from 3DM Inc (Cambridge Mass.).

Seeding of the cells on the scaffolds is also a critical step in the establishment of the bioreactor stem and/or progenitor cell culture. Since it has been observed that the initial distribution of cells within the scaffold after seeding is related to the cell densities subsequently achieved, methods of cell seeding require careful consideration. Thus, cells can be seeded in a scaffold by static loading, or, more preferably, by seeding in stirred flask bioreactors (scaffold is typically suspended from a solid support), in a rotating wall vessel, or using direct perfusion of the cells in medium in a bioreactor. Highest cell density throughout the scaffold is achieved by the latter (direct perfusion) technique.

Therapeutic compounds can also be incorporated into the scaffold material. Campbell et al (US Patent Application No. 20030125410) which is incorporated by reference as if fully set forth by reference herein, discloses methods for fabrication of 3D scaffolds for stem cell growth, the scaffolds having preformed gradients of therapeutic compounds such as analgesics, growth factors, cytokines, immune modulators, etc. The scaffold materials, according to Campbell et al, fall within the category of “bio-inks”. Such “bio-inks” are suitable for use with the bioreactors and methods of the present invention. Frondoza et al (U.S. Pat. No. 6,662,805, and US Patent Application No. 20010014475, which is incorporated by reference as if fully set forth by reference herein) have disclosed methods for the in-vitro preparation of implantable tissue replacements grown from stem and other cells, on microcarriers. According to the detailed description of their preparation and use, the microcarriers, or porous supports, can also incorporate hydrogels.

Typically the scaffold is formed by extruding a biocompatible polymer dissolved in a suitable solvent or melted to form a viscous solution from which a continuous fiber may be drawn. The solution is extruded under pressure and fed at a certain rate through an opening or openings in a dispenser of a predetermined size to form a fiber or fibers. A desired fiber thickness, typically from about <1 to about 100 microns, preferably from about 3 to about 30 microns, is formed and drawn by the actions of a moveable table having three degrees of freedom of movement that is controlled by using computer assisted design (CAD) software. The table is capable of motion in two or three planes. The rate of elongation and stretch of the fiber, if any, is similarly regulated by the programmed motion of the table in relation to the spinneret. Scaffold materials are readily available to one of ordinary skill in the art, usually in the form of a solution (suppliers are, for example, BDH, United Kingdom, and Pronova Biomedical Technology a.s. Norway). For a general overview of the selection and preparation of scaffolding materials, see the American National Standards Institute publication No. F2064-00 entitled Standard Guide for Characterization and Testing of Alginates as Starting Materials Intended for Use in Biomedical and Tissue Engineering Medical Products Applications”.

Preparation of scaffold material varies with the desired character of the scaffold. Scaffold material may comprise natural or synthetic organic polymers that can be gelled, or polymerized or solidified (e.g., by aggregation, coagulation, hydrophobic interactions, or cross-linking) into a 3-D open-lattice structure that entraps water or other molecules, e.g., to form a hydrogel. Structural scaffold materials may comprise a single polymer or a mixture of two or more polymers in a single composition. Additionally, two or more structural scaffold materials may be co-deposited so as to form a polymeric mixture at the site of deposition. Polymers used in scaffold material compositions may be biocompatible, biodegradable and/or bioerodible and may act as adhesive substrates for cells. In exemplary embodiments, structural scaffold materials are easy to process into complex shapes and have a rigidity and mechanical strength suitable to maintain the desired shape under in vivo conditions.

In certain embodiments, the structural scaffold materials may be non-resorbing or non-biodegradable polymers or materials. Such non-resorbing scaffold materials may be used to fabricate materials which are designed for long term or permanent implantation into a host organism. In exemplary embodiments, non-biodegradable structural scaffold materials may be biocompatible. Examples of biocompatible non-biodegradable polymers which are useful as scaffold materials include, but are not limited to, polyethylenes, polyvinyl chlorides, polyamides such as nylons, polyesters, rayons, polypropylenes, polyacrylonitriles, acrylics, polyisoprenes, polybutadienes and polybutadiene-polyisoprene copolymers, neoprenes and nitrile rubbers, polyisobutylenes, olefinic rubbers such as ethylene-propylene rubbers, ethylene-propylene-diene monomer rubbers, and polyurethane elastomers, silicone rubbers, fluoroelastomers and fluorosilicone rubbers, homopolymers and copolymers of vinyl acetates such as ethylene vinyl acetate copolymer, homopolymers and copolymers of acrylates such as polymethylmethacrylate, polyethylmethacrylate, polymethacrylate, ethylene glycol dimethacrylate, ethylene dimethacrylate and hydroxymethyl methacrylate, polyvinylpyrrolidones, polyacrylonitrile butadienes, polycarbonates, polyamides, fluoropolymers such as polytetrafluoroethylene and polyvinyl fluoride, polystyrenes, homopolymers and copolymers of styrene acrylonitrile, cellulose acetates, homopolymers and copolymers of acrylonitrile butadiene styrene, polymethylpentenes, polysulfones, polyesters, polyimides, polyisobutylenes, polymethylstyrenes, and other similar compounds known to those skilled in the art. Other biocompatible nondegradable polymers that are useful in accordance with the present disclosure include polymers comprising biocompatible metal ions or ionic coatings which can interact with DNA. Such metal ions include, but are not limited to gold and silver ions, Al, Fe, Mg, and Mn.

In other embodiments, the structural scaffold materials may be a “bioerodible” or “biodegradable” polymer or material. Such bioerodible or biodegradable scaffold materials may be used to fabricate temporary structures. In exemplary embodiments, biodegradable or bioerodible structural scaffold materials may be biocompatible. Examples of biocompatible biodegradable polymers which are useful as scaffold materials include, but are not limited to, polylactic acid, polyglycolic acid, polycaprolactone, and copolymers thereof, polyesters such as polyglycolides, polyanhydrides, polyacrylates, polyalkyl cyanoacrylates such as n-butyl cyanoacrylate and isopropyl cyanoacrylate, polyacrylamides, polyorthoesters, polyphosphazenes, polypeptides, polyurethanes, polystyrenes, polystyrene sulfonic acid, polystyrene carboxylic acid, polyalkylene oxides, alginates, agaroses, dextrins, dextrans, polyanhydrides, biopolymers such as collagens and elastin, alginates, chitosans, glycosaminoglycans, and mixtures of such polymers. In still other embodiments, a mixture of non-biodegradable and bioerodible and/or biodegradable scaffold materials may be used to form a biomimetic structure of which part is permanent and part is temporary.

In certain embodiments, the structural scaffold material composition is solidified or set upon exposure to a certain temperature; by interaction with ions, e.g., copper, calcium, aluminum, magnesium, strontium, barium, tin, and di-, tri- or tetra-functional organic cations, low molecular weight dicarboxylate ions, sulfate ions, and carbonate ions; upon a change in pH; or upon exposure to radiation, e.g., ultraviolet or visible light. In an exemplary embodiment, the structural scaffold material is set or solidified upon exposure to the body temperature of a mammal, e.g., a human being. The scaffold material composition can be further stabilized by cross-linking with a polyion.

In an exemplary embodiment, scaffold materials may comprise naturally occurring substances, such as, fibrinogen, fibrin, thrombin, chitosan, collagen, alginate, poly (N-isopropylacrylamide), hyaluronate, albumin, collagen, synthetic polyamino acids, prolamines, polysaccharides such as alginate, heparin, and other naturally occurring biodegradable polymers of sugar units.

In certain embodiments, structural scaffold materials may be ionic hydrogels, for example, ionic polysaccharides, such as alginates or chitosan. Ionic hydrogels may be produced by cross-linking the anionic salt of alginic acid, a carbohydrate polymer isolated from seaweed, with ions, such as calcium cations. The strength of the hydrogel increases with either increasing concentrations of calcium ions or alginate. For example, U.S. Pat. No. 4,352,883 describes the ionic cross-linking of alginate with divalent cations, in water, at room temperature, to form a hydrogel matrix. In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly (phosphazenes), poly (acrylic acids), and poly (methacrylic acids). Examples of acidic groups include carboxylic acid groups, sulfonic acid groups, and halogenated (preferably fluorinated) alcohol groups. Examples of polymers with basic side groups that can react with anions are poly (vinyl amines), poly (vinyl pyridine), and poly (vinyl imidazole). Polyphosphazenes are polymers with backbones consisting of nitrogen and phosphorous atoms separated by alternating single and double bonds. Each phosphorous atom is covalently bonded to two side chains. Polyphosphazenes that can be used have a majority of side chains that are acidic and capable of forming salt bridges with di- or trivalent cations. Examples of acidic side chains are carboxylic acid groups and sulfonic acid groups. Bioerodible polyphosphazenes have at least two differing types of side chains, acidic side groups capable of forming salt bridges with multivalent cations, and side groups that hydrolyze under in vivo conditions, e.g., imidazole groups, amino acid esters, glycerol, and glucosyl. Bioerodible or biodegradable polymers, i.e., polymers that dissolve or degrade within a period that is acceptable in the desired application (usually in vivo therapy), will degrade in less than about five years or in less than about one year, once exposed to a physiological solution of pH 6-8 having a temperature of between about 25.degree. C. and 38.degree. C. Hydrolysis of the side chain results in erosion of the polymer. Examples of hydrolyzing side chains are unsubstituted and substituted imidizoles and amino acid esters in which the side chain is bonded to the phosphorous atom through an amino linkage.

Methods for synthesis and the analysis of various types of polyphosphazenes are described in U.S. Pat. Nos. 4,440,921, 4,495,174, and 4,880,622. Methods for the synthesis of the other polymers described above are known to those skilled in the art. See, for example Concise Encyclopedia of Polymer Science and Polymeric Amines and Ammonium Salts, E. Goethals, editor (Pergamen Press, Elmsford, N.Y. 1980). Many polymers, such as poly (acrylic acid), alginates, and PLURONICS.TM., are commercially available. Water soluble polymers with charged side groups are cross-linked by reacting the polymer with an aqueous solution containing multivalent ions of the opposite charge, either multivalent cations if the polymer has acidic side groups, or multivalent anions if the polymer has basic side groups. Cations for cross-linking the polymers with acidic side groups to form a hydrogel include divalent and trivalent cations such as copper, calcium, aluminum, magnesium, and strontium. Aqueous solutions of the salts of these cations are added to the polymers to form soft, highly swollen hydrogels and membranes. Anions for cross-linking the polymers to form a hydrogel include divalent and trivalent anions such as low molecular weight dicarboxylate ions, terepthalate ions, sulfate ions, and carbonate ions. Aqueous solutions of the salts of these anions are added to the polymers to form soft, highly swollen hydrogels and membranes, as described with respect to cations. Also, a variety of polycations can be used to complex and thereby stabilize the polymer hydrogel into a semi-permeable surface membrane. An example of one polycation is poly-L-lysine. There are also natural polycations such as the polysaccharide, chitosan.

Hariri et al (US Patent Application No. 20040048796, which is incorporated by reference as if fully set forth by reference herein) teach the use of a collagen bio-fabric made from decellularized placental membranes as carriers and substrate for ex-vivo growth of stem and other cells. This collagen biofabric has high biological compatibility and the placental membranes are abundantly available. Such a bio-fabric can also be suitable for use in the methods of the present invention.

Growth and expansion of self-renewing of stem and/or progenitor cells in the conditions encountered in the bioreactor environment is influenced by numerous physicochemical parameters, such as oxygen tension, pH, osmolality, etc. Cells experience growth inhibition at high oxygen concentrations, and anoxia at lower concentrations. Bone marrow in-vivo oxygen tensions are normally 2-7%, and it has been demonstrated that a higher (15-20%) oxygen tension contributes to differentiation of hematopoietic stem cells. The role of oxygen tension in best illustrated in the direction of hematopoietic differentiation (McAdams et al. 1996a; McAdams et al. 1996b). Low oxygen concentrations promote proliferation of colony-forming cells, perhaps by augmenting the effects of growth factors such as Epo while lessening oxidative damage. Modulation of oxygen levels thus pose a serious challenge to cell culture efforts due to the combined necessity of accurate measurement as well as oxygen flow control. Low oxygen concentration (hypoxia) was recently found to favor renewal and proliferation of hematopoietic stem cells (Danet et al. 2003). Thus, in one embodiment, the oxygen tension of the bioreactor environment is about 1%-10%. In a preferred embodiment, the oxygen tension of the bioreactor environment is about 5%.

Optimum pH conditions vary with respect to different cell lineages. Low pH levels (<6.7) do not allow any hematopoietic proliferation, with erythroid differentiation specifically requiring a minimal level of pH 7.1. Optimal pH levels were found to be 7.2-7.4 for proliferation of GM-CSF, and 7.6 for erythroid cells (McAdams et al. 1996a). Also, pH 7.35-7.40 promotes differentiation, maturation and apoptosis of Mk cells, whereas lower pH (7.1) extends the expansion of the primitive Mk progenitor cells. The pH can have a further impact on growth and proliferation of stem and/or progenitor cells, corresponding closely to internal calcium concentrations that are essential for proper development. Thus, in one embodiment, the pH of the bioreactor is about 6.8-7.4.

Osmolality is another critical condition to be monitored and controlled, where possible, in the bioreactor. An optimal range for culturing of mononuclear and CD34+ cells was recently described between 0.31 and 0.32 mOsmol/kg (Noll et al. 2002). The CD34+ population shows extreme sensitivity to osmolality (beyond the linear effects seen on the MNC). In addition, Osmolality, like pH, can be an efficient modulator of lineage-specific differentiation, as progenitors of granulocytic and macrophages peak at hypotonic osmolalities (0.29 mOsmol/kg), while BFU-E proliferation is enhanced at hypertonic levels (0.34 mOsmol/kg).

Due to their anchorage-independent characteristics, hematopoietic cells of all lineages have been shown to grow exceedingly well in stirred culture systems (unlike fibroblasts and endothelial cells), potentially due to the elimination of diffusion and gradient limitations imposed by the static systems. In contrast, cellular sensitivity to shearing is most pronounced for hematopoietic cells, being able to withstand only about 30 revolutions per minute (Collins et al. 1998a). In addition, it is an accepted fact that stirring modulates the physical and metabolic characteristics of the culture, as changes in surface marker expression has been observed with such cultures (McDowell and Papoutsakis 1998). Interestingly, this provides yet an additional method for controlling the eventual fate of cells in the cultures.

As used herein, the phrase “stem cells” refers to pluripotent cells that, given the right growth conditions, may develop to any cell lineage present in the organism from which they were derived. The phrase, as used herein, refers both to the earliest renewable cell population responsible for generating cell mass in a tissue or body and the very early progenitor cells, which are somewhat more differentiated, yet are not committed and can readily revert to become a part of the earliest renewable cell population. Methods of ex-vivo culturing stem cells of different tissue origins are well known in the art of cell culturing. To this effect, see for example, the text book “Culture of Animal Cells—A Manual of Basic Technique” by Freshney, Wiley-Liss, N. Y. (1994), Third Edition, the teachings of which are hereby incorporated by reference.

As used herein, the phrase “embryonic stem (ES) cell” is defined as an undifferentiated pluripotent cell derived from the inner cell mass of blastocyst stage embryos which can grow indefinitely in culture while retaining a normal karyotype.

As used herein, the phrase “mesenchymal stem cell (MSC)” is defined as the formative pluripotential blast cell found inter alia in bone marrow, blood, dermis and periosteum that is capable of differentiating into more than one specific type of mesenchymal or connective tissue (i.e. the tissues of the body that support the specialized elements; e.g. adipose, osseous, stroma, cartilaginous, elastic and fibrous connective tissues) depending upon various influences from bioactive factors, such as cytokines. The potential to differentiate into cells such as osteoblasts and chondrocytes is retained after isolation and expansion in culture; differentiation occurs when the cells are induced in vitro under specific conditions or placed in vivo at the site of damaged tissue.

Epitopes on the surface of the human mesenchymal stem cells (hMSCs) are reactive with certain monoclonal antibodies known as SH2, SH3 and SH4 described in U.S. Pat. No. 5,486,359. These antibodies can be used as reagents to screen and capture the mesenchymal stem cell population from a heterogeneous cell population, such as exists, for example, in bone marrow.

As used herein, the phrase “endothelial stem cell (ESC)” or “endothelial progenitor cell” is defined as the stem or progenitor cell, found in various embryonic and adult tissues, including bone marrow, that is capable of neovascular engraftment, differentiating into endothelial cells, and giving rise to vascular structures such as arterioles, venules, lymphatics, etc.

Endothelial stem/progenitor cells have been characterized by a unique array of surface markers, such as CD34+, CD133+, KDR+ (Moore, J Clin Invest 2002; 109:313-15) and CD34+, CD133+, KDR+, Flk+, VE-cadherin+ (Reyes, et al J Clin Invest, 2002: 109:337-46).

As used herein the term “inhibiting” refers to slowing, decreasing, delaying, preventing or abolishing.

As used herein the term “differentiation” refers to relatively generalized or specialized changes during development. Cell differentiation of various lineages is a well-documented process and requires no further description herein. As used herein the term differentiation is distinct from maturation which is a process, although some times associated with cell division, in which a specific cell type mature to function and then dies, e.g., via programmed cell death.

The phrase “cell expansion” is used herein to describe a process of cell proliferation substantially devoid of cell differentiation. Cells that undergo expansion hence maintain their cell renewal properties and are oftentimes referred to herein as renewable cells, e.g., renewable stem cells.

As used herein the term “ex-vivo” refers to a process in which cells are removed from a living organism and are propagated outside the organism (e.g., in a test tube). As used herein, the term “ex-vivo”, however, does not refer to a process by which cells known to propagate only in-vitro, such as various cell lines (e.g., HL-60, MEL, HeLa, etc.) are cultured. In other words, cells expanded ex-vivo according to the present invention do not transform into cell lines in that they eventually undergo differentiation.

Providing the ex-vivo grown cells with conditions for ex-vivo cell proliferation include providing the cells with nutrients and preferably with one or more cytokines, as is further detailed hereinunder.

Ex-vivo expansion of the stem and/or progenitor cells, under conditions substantially inhibiting differentiation, has been described. PCT IL03/00064 to Peled et al, which is incorporated by reference as if fully set forth herein, teaches methods of reducing expression and/or activity of CD38 in cells, methods of reducing capacity of cells in responding to signaling pathways involving CD38 in the cells, methods of reducing capacity of cells in responding to retinoic acid, retinoids and/or Vitamin D in the cells, methods of reducing the capacity of cells in responding to signaling pathways involving the retinoic acid receptor, the retinoid X receptor and/or the Vitamin D receptor in the cells, methods of reducing the capacity of cells in responding to signaling pathways involving PI 3-kinase, conditions wherein cells are cultured in the presence of nicotinamide, a nicotinamide analog, a nicotinamide or a nicotinamide analog derivative or a nicotinamide or a nicotinamide analog metabolite and conditions wherein cells are cultured in the presence of a PI 3-kinase inhibitor.

In one embodiment of the invention, reducing the activity of CD38 is effected by providing the cells with an agent that inhibits CD38 activity (i.e., a CD38 inhibitor).

As used herein a “CD38 inhibitor” refers to an agent which is capable of down-regulating or suppressing CD38 activity in stem cells.

A CD38 inhibitor according to this aspect of the present invention can be a “direct inhibitor” which inhibits CD38 intrinsic activity or an “indirect inhibitor” which inhibits the activity or expression of CD38 signaling components (e.g., the cADPR and ryanodine signaling pathways) or other signaling pathways which are effected by CD38 activity.

According to presently known embodiments of this aspect of the present invention, nicotinamide is a preferred CD38 inhibitor.

Hence, in one embodiment, the method according to this aspect of the present invention is effected by providing the cells either with nicotinamide itself, or with a nicotinamide analog, a nicotinamide or a nicotinamide analog derivative or a nicotinamide or a nicotinamide analog metabolite.

As used herein, the phrase “nicotinamide analog” refers to any molecule that is known to act similarly to nicotinamide. Representative examples of nicotinamide analogs include, without limitation, benzamide, nicotinethioamide (the thiol analog of nicotinamide), nicotinic acid and α-amino-3-indolepropionic acid.

The phrase “a nicotinamide or a nicotinamide analog derivative” refers to any structural derivative of nicotinamide itself or of an analog of nicotinamide. Examples of such derivatives include, without limitation, substituted benzamides, substituted nicotinamides and nicotinethioamides and N-substituted nicotinamides and nicotinthioamides.

The phrase “a nicotinamide or a nicotinamide analog metabolite” refers to products that are derived from nicotinamide or from analogs thereof such as, for example, AND, NADH and NADPH.

Alternatively, a CD38 inhibitor according to this aspect of the present invention can be an activity neutralizing antibody which binds for example to the CD38 catalytic domain, thereby inhibiting CD38 catalytic activity. It will be appreciated, though, that since CD38 is an intracellular protein measures are taken to use inhibitors which may be delivered through the plasma membrane. In this respect a fragmented antibody such as a Fab fragment (described hereinunder) is preferably used.

The term “antibody” as used in this invention includes intact molecules as well as functional fragments thereof, such as Fab, F(ab′)₂, and Fv that are capable of binding to macrophages. These functional antibody fragments are defined as follows:
Fab, the fragment which contains a monovalent antigen-binding fragment of an antibody molecule, can be produced by digestion of whole antibody with the enzyme papain to yield an intact light chain and a portion of one heavy chain;
Fab′, the fragment of an antibody molecule that can be obtained by treating whole antibody with pepsin, followed by reduction, to yield an intact light chain and a portion of the heavy chain; two Fab′ fragments are obtained per antibody molecule;
(Fab′)_{2,} the fragment of the antibody that can be obtained by treating whole antibody with the enzyme pepsin without subsequent reduction; F(ab′)₂ is a dimer of two Fab′ fragments held together by two disulfide bonds;
Fv, defined as a genetically engineered fragment containing the variable region of the light chain and the variable region of the heavy chain expressed as two chains; and
Single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988, incorporated herein by reference).

Antibody fragments according to the present invention can be prepared by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins recipient antibody in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Inmunol. 13 65-93 (1995).

Alternatively, the method according to this aspect of the present invention can be effected by providing the ex-vivo cultured stem cells with an agent that downregulates CD38 expression.

An agent that downregulates CD38 expression refers to any agent which affects CD38 synthesis (decelerates) or degradation (accelerates) either at the level of the mRNA or at the level of the protein. For example, a small interfering polynucleotide molecule which is designed to down regulate the expression of CD38 can be used according to this aspect of the present invention.

An example for a small interfering polynucleotide molecule which can downregulate the expression of CD38 is a small interfering RNA or siRNA, such as, for example, the morpholino antisense oligonucleotides described by in Munshi et al. (Munshi C B, Graeff R, Lee H C, J Biol Chem 2002 Dec. 20; 277(51):49453-8), which includes duplex oligonucleotides which direct sequence specific degradation of mRNA through the previously described mechanism of RNA interference (RNAi) (Hutvagner and Zamore (2002) Curr. Opin. Genetics and Development 12:225-232).

As used herein, the phrase “duplex oligonucleotide” refers to an oligonucleotide structure or mimetics thereof, which is formed by either a single self-complementary nucleic acid strand or by at least two complementary nucleic acid strands. The “duplex oligonucleotide” of the present invention can be composed of double-stranded RNA (dsRNA), a DNA-RNA hybrid, single-stranded RNA (ssRNA), isolated RNA (i.e., partially purified RNA, essentially pure RNA), synthetic RNA and recombinantly produced RNA.

Preferably, the specific small interfering duplex oligonucleotide of the present invention is an oligoribonucleotide composed mainly of ribonucleic acids.

Instructions for generation of duplex oligonucleotides capable of mediating RNA interference are provided in www.ambion.com.

Hence, the small interfering polynucleotide molecule according to the present invention can be an RNAi molecule (RNA interference molecule).

Alternatively, a small interfering polynucleotide molecule can be an oligonucleotide such as a CD38-specific antisense molecule or a rybozyme molecule, further described hereinunder.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art.

Oligonucleotides used according to this embodiment of the present invention are those having a length selected from a range of 10 to about 200 bases preferably 15-150 bases, more preferably 20-100 bases, most preferably 20-50 bases.

The oligonucleotides of the present invention may comprise heterocyclic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder. Such modifications can oftentimes facilitate oligonucleotide uptake and resistivity to intracellular conditions.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos.: ,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No: 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No: 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No: 6,303,374.

It is not necessary for all positions in a given oligonucleotide molecule to be uniformly modified, and in fact more than one of the aforementioned modifications may be incorporated in a single compound or even at a single nucleoside within an oligonucleotide.

As described hereinabove, the oligonucleotides of the present invention are preferably antisense molecules, which are chimeric molecules. “Chimeric antisense molecules” are oligonucleotides, which contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target polynucleotide. An additional region of the oligonucleotide may serve as a substrate for enzymes capable of cleaving RNA:DNA or RNA:RNA hybrids. An example for such includes RNase H, which is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense molecules of the present invention may be formed as composite structures of two or more oligonucleotides, modified oligonucleotides, as described above. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein fully incorporated by reference.

The oligonucleotides of the present invention can further comprise a ribozyme sequence. Ribozymes are being increasingly used for the sequence-specific inhibition of gene expression by the cleavage of mRNAs. Several rybozyme sequences can be fused to the oligonucleotides of the present invention. These sequences include but are not limited ANGIOZYME specifically inhibiting formation of the VEGF-R (Vascular Endothelial Growth Factor receptor), a key component in the angiogenesis pathway, and HEPTAZYME, a rybozyme designed to selectively destroy Hepatitis C Virus (HCV) RNA, (Rybozyme Pharmaceuticals, Incorporated—WEB home page).

Further alternatively, a small interfering polynucleotide molecule, according to the present invention can be a DNAzyme.

DNAzymes are single-stranded catalytic nucleic acid molecules. A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M Curr Opin Mol Ther 2002;4:119-21).

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al. , 20002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

Alternatively, as described hereinabove, retinoid receptor superfamily inhibitors (e.g., antagonists, siRNA molecules, antisense molecules, antibodies, etc.) which downregulate or suppress retinoid receptor activity and/or expression can be used to down regulate CD38 expression.

Briefly, retinoid receptors such as RAR, RXR and VDR have been reported to be involved in the regulation of gene expression pathways associated with cell proliferation and differentiation and in particular in the regulation of CD38 expression. Hence, preferred agents that downregulate CD38 expression according to the present invention include RAR antagonists, RXR antagonists and VDR antagonists or, alternatively, antagonists for reducing the capacity of the stem cells in responding to retinoic acid, retinoid and/or Vitamin D.

As used herein the term “antagonist” refers to an agent that counteracts or abrogates the effects of an agonist or a natural ligand of a receptor. Further features relating to such antagonists are detailed hereinunder.

In one preferred embodiment, reducing the capacity of the stem cells in responding to the above antagonists and/or signaling pathways of the above receptors and kinase is by ex-vivo culturing the stem cells in a presence of an effective amount of at least one retinoic acid receptor antagonist, at least one retinoid X receptor antagonist and/or at least one Vitamin D receptor antagonist, preferably, for a time period of 0.1-50%, preferably, 0.1-25%, more preferably, 0.1-15%, of an entire ex-vivo culturing period of the stem cells or for the entire period. In this respect it was surprisingly uncovered that an initial pulse exposure to an antagonist is sufficient to exert cell expansion long after the antagonist was removed from the culturing set up.

Many antagonists to RAR, RXR and VDR are presently known, some of which are listed hereinafter.

The retinoic acid receptor antagonist used in context of the different aspects and embodiments of the present invention can be: AGN 194310; AGN 109; 3-(4-Methoxy-phenylsulfanyl)-3-methyl-butyric acid; 6-Methoxy-2,2-dimethyl-thiochroman-4-one,2,2-Dimethyl-4-oxo-thiochroman-6-yltrifluoromethane-sulfonate; Ethyl 4-((2,2 dimethyl-4-oxo-thiochroman-6-yl)ethynyl)-benzoate; Ethyl 4-((2,2-dimethy 1-4-triflouromethanensulfonyloxy -(2H)-thiochromen-6-yl)ethynyl)-benzoate(41); Thiochromen-6-yl]-ethynyl]-benzoate(yl); (p-[(E)-2-[3′4′-Dihydro-4,4′-dimethyl-7′-(heptyloxy)-2′H-1-benzothiopyran-6′yl] propenyl] benzoic acid 1′1′-dioxide; 2E,4E,6E-[7-(3,5-Di-t-butyl-4-n-butoxyphenyl)-3-methyl]-octa-2,4,6-trienoic acid; 2E,4E,6E-[7-(3,5-Di-t-butyl-4-n-propoxyphenyl)-3-methyl]-octa-2,4,6-trienoic acid; 2E,4E,6E-[7-(3,5-Di-t-butyl-4-n-pentoxyphenyl)-3-methyl]-octa-2,4,6-trienoic acid; 2E,4E,6E-[7-(3,5-Di-t-butyl-4-n-hexoxyphenyl)-3-methyl]-octa-2,4,6-trienoic acid; 2E,4E,6E-[7-(3,5-Di-t-butyl-4-n-heptoxyphenyl)-3-methyl]-octa-2,4,6-trienoic acid; 2E,4E,6E-[7-(3,5-Di-t-butyl-4-n-octoxyphenyl)-3-methyl]-octa-2,4,6-trienoic acid; (2E,4E,6E)-7-[3-t-butyl-5-(1-phenyl-vinyl)-phenyl]-3-methyl-octa-2,4,6-trienoic acid; 2E,4E,6E-[7-(3,5-Di-t-butyl-4{[4,5-.sup.3 H.sub.2]-n-pentoxy}phenyl)-3-methyl]-octa-2,4,6-trienoic acid; (2E,4E)-(1 RS,2RS)-5-[2-(3,5-di-tert.butyl-2-ethoxy-phenyl)-cyclopropyl]-3-methyl-penta-2,4-dienoic acid ethyl ester; (2E,4E)-(1RS,2RS)-5-[2-(3,5-di-tert.butyl-2-ethoxy-phenyl)-cyclopropyl]-3-methyl-penta-2,4-dienoic acid; (2E,4E)-(1RS,2RS)-5-[2-(3,5-di-tert.butyl-2-butoxy-phenyl)-cyclopropyl]-3-methyl-penta-2,4-dienoic acid; (2E,4E,6Z)-7-[3,5-di-tert.butyl-2-ethoxyphenyl]3-methyl-2,4,6-octatrienoic acid; (2E,4E,6Z)-7-[3,5-di-tert.butyl-2-butyloxyphenyl]-3-methyl-2,4,6-octatrienoic acid; 4-(5,6,7,8-tetrahydro-5,5,8,8-tetramethyl-2-naphthalene-carboxamido) benzoic acid; (2E,4E)-3-methyl-5-[(1S,2S)-2-(5,5,8,8-tetramethyl-5,6,7,8-tetrahydro-naphthalen-2-yl)-cyclopropyl]-penta-2,4-dienoic acid; p-[(E)-2-[3′4′-Dihydro-4′,4′-dimethyl-7′-(heptyloxy)-2′H-1-benzothiopyran-6′-yl]propenyl]benzoic acid; 1′,1′-dioxide, 4-(7,7,10,10-Tetramethyl-1-pyridin-3-ylmethyl-4,5,7,8,9,10-hexahydro-1H-naphto[2,3-g]indol-3-yl)-benzoic acid; (2E,4E,6Z)-7-[3,5-di-tert.butyl-2-methoxyphenyl]-3-methyl-2,4,6-octatrienoic acid; (2E,4E,6Z)-7-[3,5-di-tert.butyl-2-ethoxyphenyl]-3-methyl-2,4,6-octatrienoic acid; (2E,4E,6Z)-7-[3,5-di-tert.butyl-2-hexyloxyphenyl]-3-methyl-2,4,6-octatrienoic acid; (2E,4E,6Z)-7-[3,5-di-tert.butyl-2-octyloxyphenyl]-3-methyl-2,4,6-octatrienoic acid; and (2E,4E)-(1RS,2RS)-5-[2-(3,5-di-tert-butyl-2-butoxy-phenyl)-cyclopropyl]-3-methyl-penta-2,4-dienoic acid (2E,4E,6Z)-7-(3-n-propoxy-5,6,7,8-tetrahydro-5,5,8,8-tetramethylnaphthalene-2-yl)-3-methylocta-2,4,6-trienoic acid, and 4-(5H-2,3(2,5 dimethyl-2,5-hexano)-5-n-propyldibenzo[b,e][1,4]diazepin-11-yl)benzoic acid, and 4-(5H-2,3-(2,5-dimethyl-2,5-hexano)-5methyl-8-nitrodibenzo[b,e][1,4]diazepin-11-yl)benzoic acid, and 4-{[4-(4-Ethylphenyl)2,2-dimethyl-(2H)-thiochromen-6-yl]ethynyl}benzoic acid, and 4-[4-2methyl-1,2-dicarba-closo-dodecaboran-1-yl-phenylcarbamoyl]benzoic acid, and 4-[4,5,7,8,9,10-hexahydro-7,7,10,10-tetramethyl-1-(3-pyridylmethyl)-anthra[1,2-b]pyrrol-3-yl]benzoic acid, and (3-pyridylmethyl)-]5-thiaanthra[2,1-b]pyrrol-3-yl)benzoic acid, and (3-pyridylmethyl)-anthra[2ml-d]pyrazol-3-yl]benzoic acid.

The retinoid X receptor antagonist used in context of the different aspects and embodiments of the present invention can be: LGN100572, 1-(3-hydroxy-5,6,7,8-tetrahydro-5,5,8,8-tetramethylnaphthalene-2-yl)ethanone, 1-(3-propoxy-5,6,7,8-tetrahydro-5,5,8,8-tetramethylnaphthalene-2-yl)ethanone, 3-(3-propoxy-5,6,7,8-tetrahydro-5,5,8,8-tetramethylnaphthalene-2-yl)but-2-enenitrile, 3-(3-propoxy-5,6,7,8-tetrahydro-5,5,8,8-tetramethylnaphthalene-2-yl)but-2-enal, (2E,4E,6E)-7-3[-propoxy-5,6,7,8-tetrahydro 5,5,8,8-tetramethyl-2-naphthalene-2-yl]-3-methylocta-2,4,6-trienoic acid, 4-[3,5,5,8,8-pentamethyl-5,6,7,8-tetrahydro-2-naphthyl)carbonyl] benzoic acid, 4-[1-(3,5, 5,8,8-pentamethyl-5,6,7,8-tetrahydro-2-naphthyl)ethenyl] benzoic acid, 4-[1(3,5,5,8,8-pentamethyl-5,6,7,8-tetrahydro-2-naphthyl)cyclopropyl] benzoic acid, 4-[1-(3,5,5,8,8-pentamethyl-5,6,7,8-tetrahydro-2-naphthyl)ethenyl] benzenete trazole, 2-[1-(5,5,8,8-tetramethyl-5,6,7,8-tetrahydro-2-naphthyl) ethenyl]pyridine-5-carboxylic acid, 2-[1-(3,5,5,8,8-pentamethyl-5,6,7,8-tetrahydro-2-naphthyl)ethyl]pyridine-5-carboxylic acid, ethyl-2-[1-(3,5,5,8, 8-pentamethyl-5,6,7,8-tetrahydro-2-naphthyl)ethenyl]pyridine-5-carboxylate, 5-[1-3,5,5,8,8-pentamethyl-5,6,7,8-tetrahydro-2-naphthyl)ethenyl]pyridine-2-carboxylic acid, 2-[1-(3,5,5,8,8-pentamethyl-5,6,7,8-tetrahydro-2-naphthyl) cyclopropyl]pyridine-5-carboxylic acid, methyl 2-[1-(3,5,5,8,8-pentamethyl-5,6,7,8-tetrahydro-2-naphthyl)cyclopropyl]pyridine-5-carboxylate, 4-[1-(3,5, 5,8,8-pentamethyl-5,6,7,8-tetrahydro-2-naphthyl)ethenyl]-N-(4-hydroxyphenyl) benzamide, 2-[1-(3,5,5,8,8-Pentamethyl-5,6,7,8-tetrahydro-2-naphthyl) ethenyl] pyridine-5-carboxylic acid, 2-[1-(3,5,5,8,8-Pentamethyl-5, 6,7,8-tetrahydro-2-naphthyl)cyclopropyl]pyridine-5-carboxylic acid, 4-[(3,5, 5,8,8-pentamethyl-5,6,7,8-tetrahydro-2-naphthyl)carbonyl]benzoic acid butyloxime, 4-[(3,5,5,8,8-pentamethyl-5,6,7,8-tetrahydro-2-naphthyl) carbonyl]benzoic acid propyloxime, 4-[(3,5,5,8,8-pentamethyl-5,6,7,8-terrahydro-2-naphthyl)carbonyl]benzoic acid cyanoimine, 4-[(3,5,5,8,8-pentamethyl-5,6,7,8-tetrahydro-2-naphthyl)carbonyl]benzoic acid allyloxime, 4-[(3,5,5,8,8-pentamethyl-5,6,7,8-tetrahydro-2-naphthyl)carbonyl]benzoic acid 4-(3-methylbut-2-enoic acid)oxime, and 4-[(3,5,5,8,8-pentamethyl-5,6,7,8-tetrahydro-2-naphthyl)carbonyl]benzoic acid 1-aminoethyloxime (2E,4E,6Z)-7-(3-n-propoxy-5,6,7,8-tetrahydro-5,5,8,8-tetramethylnaphthalene-2-yl)-3-methylocta-2,4,6-trienoic acid, and 4-(5H-2,3(2,5 dimethyl-2,5-hexano)-5-n-propyldibenzo[b,e][1,4]diazepin-11-yl)benzoic acid, and 4-(5H-2,3-(2,5-dimethyl-2,5-hexano)-5m.

The Vitamin D receptor antagonist used in context of the different aspects and embodiments of the present invention can be: 1 alpha, 25-(OH)-D3-26,23 lactone; 1alpha, 25-dihydroxyvitamin D (3); the 25-carboxylic ester ZK159222; (23S)- 25-dehydro-1 alpha-OH-D (3); (23R)-25-dehydro-1 alpha-OH-D (3); 1 beta, 25 (OH)₂ D₃; 1 beta, 25(OH)₂-3-epi-D₃; (23S) 25-dehydro-1 alpha(OH) D3-26,23-lactone; (23R) 25-dehydro-1 alpha(OH)D3-26,23-lactone and Butyl-(5Z,7E,22E-(1S,7E,22E-(1S,3R,24R)-1,3,24-trihydroxy-26,27-cyclo-9,10-secocholesta-5,7,10(19),22-tetraene-25-carboxylate).

The above listed antagonists are known for their high affinity towards their respective cognate receptors. However, it may be possible for these molecules to be active towards other receptors.

Each of the agents described hereinabove may reduce the expression or activity of CD38 individually. However, the present invention aims to also encompass the use of any subcombination of these agents.

It will be appreciated that protein agents (e.g., antibodies) of the present invention can be expressed from a polynucleotide encoding same and provided to ex-vivo cultured stem cells employing an appropriate gene delivery vehicle/method and a nucleic acid construct as is further described hereinunder.

Examples of suitable constructs include, but are not limited to pcDNA3, pcDNA3.1 (±), pGL3, PzeoSV2 (±), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

As the method of ex-vivo expanding a population of stem cells, while at the same time, substantially inhibiting differentiation of the stem cells ex-vivo, according to this aspect of the present invention, is effected by modulating CD38 expression and/or activity, either at the protein level, using RAR, RXR or VDR antagonists or a CD38 inhibitor such as nicotinamide and analogs thereof, or at the at the expression level via genetic engineering techniques, as is detailed hereinabove, there are further provided, according to the present invention, several preferred methods of ex-vivo expanding a population of stem cells, while at the same time, substantially inhibiting differentiation of the stem cells ex-vivo.

Still alternatively, according to the present invention, as described hereinabove, inhibitors of activity or expression of PI 3-kinase are used to down regulate CD38 expression.

It will be appreciated, in the context of the present invention, that Hori et al (PNAS USA 2002;99:16105-10) reported that treatment of mouse embryonic stem cells with inhibitors of phosphoinositide 3-kinase caused differentiation of the stem cells, producing cells that resembled pancreatic β cells, which were implanted into diabetic mice for restoration of pancreas function. Thus, the prior art teaches away from the methods of the present invention.

In stark contrast, PCT IL2004/000215 to Peled et al., which is incorporated by reference as if fully set forth herein, discloses the use of inhibitors of PI 3-K activity or expression for ex-vivo expansion of stem and/or progenitor cells while inhibiting differentiation thereof.

Thus, in still another particular embodiment of this aspect of the present invention, culturing the stem and/or progenitor cells ex-vivo under conditions allowing for cell proliferation and at the same time inhibiting differentiation is effected by culturing the cells in conditions reducing the capacity of the cells in responding to signaling pathways involving PI 3-kinase, or in conditions wherein the cells are cultured in the presence of the PI 3-kinase inhibitors.

All the methodologies described herein with respect to the inhibition of expression apply also to inhibition of expression of PI 3-kinase. These methodologies include, for example, the use of polynucleotides, such as small interfering RNA molecules, antisense ribozymes and DNAzymes, as well as intracellular antibodies.

Inhibition of PI 3-kinase activity can be effected by known PI 3-kinase inhibitors, such as wortmannin and LY294002 and the inhibitors described in, for example, U.S. Pat. No. 5,378,725, which is incorporated herein by reference. In one particular, the ex-vivo expanding a population of stem cells, while at the same time, substantially inhibiting differentiation of the stem cells ex-vivo is effected by providing the stem cells with ex-vivo culture conditions for ex-vivo cell proliferation and, at the same time, for reducing a capacity of the stem cells in responding to retinoic acid, retinoids and/or Vitamin D, thereby expanding the population of stem cells while at the same time, substantially inhibiting differentiation of the stem cells ex-vivo. In still another particular embodiment of this aspect of the present invention, the ex-vivo expanding a population of stem cells, while at the same time, substantially inhibiting differentiation of the stem cells ex-vivo is effected by obtaining adult or neonatal umbilical cord whole white blood cells or whole bone marrow cells sample and providing the cells in the sample with ex-vivo culture conditions for stem cells ex-vivo cell proliferation and with a PI 3-kinase inhibitor, thereby expanding a population of a renewable stem cells in the sample.

In one preferred embodiment, concomitant with treating the cells with conditions which allow for ex-vivo the stem cells to proliferate, the cells are short-term treated or long-term treated to reduce the expression and/or activity of PI 3-kinase.

In one embodiment of the invention, reducing the activity of PI 3-kinase is effected by providing the cells with an modulator of PI 3-kinase that inhibits PI 3-kinase catalytic activity (i.e., a PI 3-kinase inhibitor).

As used herein a “modulator capable of downregulating PI 3-kinase activity or gene expression” refers to an agent which is capable of down-regulating or suppressing PI 3-kinase activity in stem cells.

An inhibitor of PI 3-kinase activity according to this aspect of the present invention can be a “direct inhibitor” which inhibits PI 3-kinase intrinsic activity or an “indirect inhibitor” which inhibits the activity or expression of PI 3-kinase signaling components (e.g., the Akt and PDK1 signaling pathways) or other signaling pathways which are effected by PI 3-kinase activity.

According to presently known embodiments of this aspect of the present invention, wortmannin and LY294002 are preferred PI 3-kinase inhibitors.

Hence, in one embodiment, the method according to this aspect of the present invention is effected by providing known PI 3-kinase inhibitors, such as wortmannin, LY294002, and active derivatives thereof, as described in, for example, U.S. Pat. Nos. 5,378,725, 5,480,906, 5,504,103, and in International Patent Publications WO 03072557, and WO 9601108, which are incorporated herein by reference, and by the specific PI 3-kinase inhibitors disclosed in US Patent Publication 20030149074 to Melese et al., also incorporated herein by reference.

Phosphatidylinositol 3-kinase inhibitors are well known to those of skill in the art. Such inhibitors include, but are not limited to Ly294002 (Calbiochem Corp., La Jolla, Calif.) and wortmannin (Sigma Chemical Co., St. Louis Mo.) which are both potent and specific PI3K inhibitors. The chemical properties of Ly294002 are described in detail in J. Biol., Chem., (1994) 269: 5241-5248. Briefly, Ly294002, the quercetin derivative, was shown to inhibit phosphatidylinositol 3-kinase inhibitor by competing for phosphatidylinositol 3-kinase binding of ATP. At concentrations at which LY294002 fully inhibits the ATP-binding site of PI3K, it has no inhibitory effect against a number of other ATP-requiring enzymes including PI4-kinase, EGF receptor tyrosine kinase, src-like kinases, MAP kinase, protein kinase A, protein kinase C, and ATPase.

LY294002 is very stable in tissue culture medium, is membrane permeable, has no significant cytotoxicity, and at concentrations at which it inhibits members of PI3K family, it has no effect on other signaling molecules.

Phosphatidylinositol 3-kinase, has been found to phosphorylate the 3-position of the inositol ring of phosphatidylinositol (PI) to form phosphatidylinositol 3-phosphate (PI-3P) (Whitman et al. (1988) Nature, 322: 664-646). In addition to PI, this enzyme also can phosphorylate phosphatidylinositol 4-phosphate and phosphatidylinositol 4,5-bisphosphate to produce phosphatidylinositol 3,4-bisphosphate and phosphatidylinositol 3,4,5-trisphosphate (PIP3), respectively (Auger et al. (1989) Cell, 57: 167-175). PI 3-kinase inhibitors are materials that reduce or eliminate either or both of these activities of PI 3-kinase. Identification, isolation and synthesis of such inhibitors is disclosed in U.S. Pat. No: 6,413,773 to Ptasznik et al.

The phrase “active derivative” refers to any structural derivative of wortmannin or LY294002 having a PI 3-kinase downregulatory activity, as measured, for example, by catalytic activity, binding studies, etc, in vivo or in vitro.

Alternatively, a modulator downregulating PI 3-kinase activity or gene expression according to this aspect of the present invention can be an activity neutralizing anti-PI 3-kinase antibody which binds, for example to the PI 3-kinase catalytic domain, or substrate binging site, thereby inhibiting PI 3-kinase catalytic activity. It will be appreciated, though, that since PI 3-kinase is an intracellular protein measures are taken to use modulators which may be delivered through the plasma membrane. In this respect a fragmented antibody such as a Fab fragment (described hereinunder), or a genetically engineered ScFv is preferably used.

A modulator that downregulates PI 3-kinase expression refers to any agent which affects PI 3-kinase synthesis (decelerates) or degradation (accelerates) either at the level of the mRNA or at the level of the protein. For example, downregulation of PI 3-kinase expression can be achieved using oligonucleotide molecules designed to specifically block the transcription of PI 3-kinase mRNA, or the translation of PI 3-kinase transcripts at the ribosome, can be used according to this aspect of the present invention. In one embodiment, such oligonucleotides are antisense oligonucleotides.

Design of antisense molecules which can be used to efficiently inhibit PI 3-kinase expression must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof Sequences suitable for use in construction and synthesis of oligonucleotides which specifically bind to PI 3-kinase mRNA, genomic DNA, promoter and/or other control sequences of PI 3-kinase are available in published PI 3-kinase nucleotide sequences, including, but not limited to, GenBank Accession Nos: AF327656 (human gamma catalytic subunit); NM006219 (human beta subunit); NM002647 (human class III); NM181524 (human p85 alpha subunit); U86453 (human p110 delta isoform); and S67334 (human p110 beta isoform).

The prior art teaches of a number of delivery strategies which can be used to efficiently deliver oligonucleotides into a wide variety of cell types (see, for example, Luft (1998) J Mol Med 76(2): 75-6; Kronenwett et al. (1998) Blood 91(3): 852-62; Rajur et al. (1997) Bioconjug Chem 8(6): 935-40; Lavigne et al. (1997) Biochem Biophys Res Commun 237(3): 566-71 and Aoki et al. (1997) Biochem Biophys Res Commun 231(3): 540-5).

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. (1999) Biotechnol Bioeng 65(1): 1-9].

Such algorithms have been successfully employed to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al. (1998) Nature Biotechnology 16, 1374-1375). Examples of antisense molecules which have been demonstrated capable of down-regulating the expression of PI 3-kinase are the PI 3-kinase specific antisense oligonucleotides described by Mood et al (Cell Signal 2004; 16:631-42), incorporated herein by reference. The production of PI 3-kinase-specific antisense molecules is disclosed by Ptasznik et al (U.S. Pat. No.: 6,413,773), incorporated herein by reference.

Reducing the capacity of the cells in responding to retinoic acid, retinoids and/or Vitamin D, or to retinoic acid, retinoid X and/or Vitamin D receptor signaling may be effected, for example, by the administration of chemical inhibitors, including receptor antagonists. In another particular, the method of ex-vivo expanding a population of stem cells, while at the same time, substantially inhibiting differentiation of the stem cells ex-vivo is effected by providing the stem cells with ex-vivo culture conditions for ex-vivo cell proliferation and, at the same time, for reducing a capacity of the stem cells in responding to signaling pathways involving the retinoic acid receptor, retinoid-X receptor and/or Vitamin D receptor, thereby expanding the population of stem cells while at the same time, substantially inhibiting differentiation of the stem cells ex-vivo. Reducing the capacity of the cells to respond to retinoic acid, retinoid X and/or Vitamin D receptor signaling events, includes treating the cells with antagonists supplied continuously or for a short-pulse period, and is effected by a diminution or abrogation of cellular signaling pathways through their respective, cognate receptors.

Final concentrations of the antagonists may be, depending on the specific application, in the micromolar or millimolar ranges. For example, within about 0.1 μM to about 100 mM, preferably within about 4 μM to about 50 mM, more preferably within about 5 μM to about 40 mM.

Final concentrations of the nicotinamide or the analogs, derivatives or metabolites thereof and of the PI 3-kinase inhibitor are preferably, depending on the specific application, in the millimolar ranges. For example, within about 0.1 mM to about 20 mM, preferably within about 1 mM to about 10 mM, more preferably within about 5 mM to about 10 mM.

In still another particular embodiment of this aspect of the present invention, culturing the stem and/or progenitor cells ex-vivo under conditions allowing for cell proliferation and at the same time inhibiting differentiation is effected by culturing the cells in the presence of a copper chelator. PCT IL99/00444 to Peled, et al, which is incorporated by reference as if fully set for herein, discloses the use of transition metal chelators, having high affinity for copper, for efficient ex-vivo expansion of stem and/or progenitor cells, while substantially inhibiting differentiation thereof.

Final concentrations of the chelator may be, depending on the specific application, in the micromolar or millimolar ranges. For example, within about 0.1 μM to about 100 mM, preferably within about 4 μM to about 50 mM, more preferably within about 5 μM to about 40 mM.

According to a preferred embodiment of the invention the chelator is a polyamine chelating agent, such as, but not limited to ethylendiamine, diethylenetriamine, triethylenetetramine, triethylenediamine, tetraethylenepentamine, aminoethylethanolamine, aminoethylpiperazine, pentaethylenehexamine, triethylenetetramine-hydrochloride, tetraethylenepentamine-hydrochloride, pentaethylenehexamine-hydrochloride, tetraethylpentamine, captopril, penicilamine, N,N′-bis(3-aminopropyl)-1,3-propanediamine, N,N,Bis (2 animoethyl) 1,3 propane diamine, 1,7-dioxa-4,10-diazacyclododecane, 1,4,8,11-tetraaza cyclotetradecane-5,7-dione, 1,4,7-triazacyclononane trihydrochloride, 1-oxa-4,7,10-triazacyclododecane, 1,4,8,12-tetraaza cyclopentadecane or 1,4,7,10-tetraaza cyclododecane, preferably tetraethylpentamine. The above listed chelators are known in their high affinity towards Copper ions.

In yet another particular embodiment of this aspect of the present invention, culturing the stem and/or progenitor cells ex-vivo under conditions allowing for cell proliferation and at the same time inhibiting differentiation is effected by culturing the cells in the presence of a copper chelate. PCT IL03/00062 to Peled, et al, which is incorporated by reference as if fully set for herein, discloses the use of copper chelates, complexes of copper and heavy metal chelators having high affinity for copper, for efficient ex-vivo expansion of stem and/or progenitor cells, while substantially inhibiting differentiation thereof.

The copper chelate, according to the present invention, is used in these and other aspects of the present invention, in the context of expanding a population of stem and/or progenitor cells, while at the same time reversibly inhibiting differentiation of the stem and/or progenitor cells. Providing the cells with the copper chelate maintains the free copper concentration available to the cells substantially unchanged.

The copper chelate according to the present invention is oftentimes capable of forming an organometallic complex with a transition metal other than copper. As metals other than copper are typically present in the cells (e.g., zinc) or can be administered to cells during therapy (e.g., platinum), it was found that copper chelates that can also interact with other metals are highly effective. Representative examples of such transition metals include, without limitation, zinc, cobalt, nickel, iron, palladium, platinum, rhodium and ruthenium.

The copper chelates of the present invention comprise copper ion (e.g., Cu⁺¹, Cu⁺²) and one or more chelator(s). As is discussed hereinabove, preferred copper chelators include polyamine molecules, which can form a cyclic complex with the copper ion via two or more amine groups present in the polyamine.

Hence, the copper chelate used in the context of the different aspects and embodiments of the present invention preferably includes a polyamine chelator, namely a polymeric chain that is substituted and/or interrupted with 1-10 amine moieties, preferably 2-8 amine moieties, more preferably 4-6 amine moieties and most preferably 4 amine moieties.

The phrases “amine moiety”, “amine group” and simply “amine” are used herein to describe a —NR′R″ group or a —NR′— group, depending on its location within the molecule, where R′ and R″ are each independently hydrogen, alkyl, cycloalkyl, aryl, heteroaryl or heterocyclic, as these terms are defined hereinbelow.

The polyamine chelator can be a linear polyamine, a cyclic polyamine or a combination thereof.

A linear polyamine, according to the present invention, can be a polyamine that has a general formula I:
HX—Am—(Y₁B₁) . . . . (YnBn)n—ZH Formula I
wherein m is an integer from 1 to 10; n is an integer from 0 to 20; X and Z are each independently selected from the group consisting of an oxygen atom, a sulfur atom and a —NH group; Y₁ and Yn are each independently selected from the group consisting of an oxygen atom, a sulfur atom and a —NH group; A is an alkylene chain having between 1 and 10 substituted and/or non-substituted carbon atoms; and B₁, and Bn are each independently an alkylene chain having between 1 and 20 substituted and/or non-substituted carbon atoms, provided that at least one of X, Z, Y₁ and Yn is a —NH group and/or at least one of the carbon atoms in the alkylene chains is substituted by an amine group.

Hence, the linear polyamine, according to the present invention, is preferably comprised of one or more alkylene chains (Am, B₁ . . . . Bn, in Formula I), is interrupted by one or more heteroatoms such as S, O and N (Y₁ . . . . Yn in Formula I), and terminates with two such heteroatoms (X and Z in Formula I).

Alkylene chain A, as is described hereinabove, includes 1-10 substituted or non-substituted carbon atoms and is connected, at least at one end thereof, to a heteroatom (e.g., X in Formula I). Whenever there are more than one alkylene chains A (in cases where m is greater than one), only the first alkylene chain A is connected to X. However, m is preferably 1 and hence the linear polyamine depicted in Formula I preferably includes only one alkylene chain A.

Alkylene chain B, as is described hereinabove, includes between 1 and 20 substituted or non-substituted carbon atoms. The alkylene chain B is connected at its two ends to a heteroatom (Y₁ . . . . Yn and Z in Formula I).

The preferred linear polyamine delineated in Formula I comprises between 1 and 20 alkylene chains B, denoted as B₁ . . . . Bn, where “B₁ . . . . Bn” is used herein to describe a plurality of alkylene chains B, namely, B₁, B₂, B₃, . . . ., Bn−1 and Bn, where n equals 0-20. These alkylene chains can be the same or different. Each of B₁ . . . . Bn is connected to the respective heteroatom Y₁ . . . . Yn, and the last alkylene chain in the structure, Bn, is also connected to the heteroatom Z.

It should be noted that herein throughout, whenever an integer equals 0 or whenever a component of a formula is followed by the digit 0, this component is absent from the structure. For example, if n in Formula I equals 0, there is no alkylene chain B and no heteroatom Y are meant to be in the structure.

Preferably, n equals 2-10, more preferably 2-8 and most preferably 3-5. Hence, the linear polyamine depicted in Formula I preferably includes between 3 and 5 alkylene chains B, each connected to 3-5 heteroatoms Y.

The linear polyamine depicted in Formula I must include at least one amine group, as this term is defined hereinabove, preferably at least two amine groups and more preferably at least four amine groups. The amine group can be present in the structure as the heteroatoms X, Z or Y₁ . . . . Yn, such that at least one of X, Z and Y₁ . . . . Yn is a —NH— group, or as a substituent of one or more of the substituted carbon atoms in the alkylene chains A and B₁ . . . . Bn. The presence of these amine groups is required in order to form a stable chelate with the copper ion, as is discussed hereinabove.

The alkylene chain A preferably has a general Formula II: 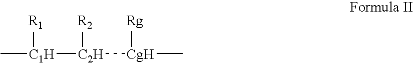
wherein g is an integer that equals 0 or 3-10.

Hence, the alkylene chain A is comprised of a plurality of carbon atoms C₁, C₂, C₃ . . . ., Cg—1 and Cg, substituted by the respective R₁, R₂, R₃. . . . Rg—1 and Rg groups. Preferably, the alkylene chain A includes 2-10 carbon atoms, more preferably, 2-6 and most preferably 24 carbon atoms.

As is defined hereinabove, in cases where g equals 0, the component CgH(Rg) is absent from the structure and hence the alkylene chain A comprises only 2 carbon atoms.

R₁, R₂ and Rg are each a substituent attached to the carbon atoms in A. Each of R₁, R₂ and Rg can independently be a substituent such as, but not limited to, hydrogen, alkyl, alkenyl, alkynyl, aryl, cycloalkyl, heteroalicyclic, heteroaryl, halo, amino, alkylamino, arylamino, cycloalkylamino, heteroalicyclic amino, heteroarylamino, hydroxy, alkoxy, aryloxy, azo, C-amido, N-amido, ammonium, thiohydroxy, thioalkoxy, thioaryloxy, sulfonyl, sulfinyl, N-sulfonamide, S-sulfonamide, phosphonyl, phosphinyl, phosphonium, carbonyl, thiocarbonyl, C-carboxy, O-carboxy, C-thiocarboxy, O-thiocarboxy, N-carbamate, O-carbamate, N-thiocarbamate, O-thiocarbamate, urea, thiourea, borate, borane, boroaza, silyl, siloxy, silaza, aquo, alcohol, peroxo, amine oxide, hydrazine, alkyl hydrazine, aryl hydrazine, nitric oxide, cyanate, thiocyanate, isocyanate, isothiocyanate, cyano, alkylnitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, carboxylic acid, alkyl carboxylic acid, aryl carboxylic acid, sulfate, sulfite, bisulfite, thiosulfate, thiosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, phosphate, thiophosphate, phosphite, pyrophosphite, triphosphate, hydrogen phosphate, dihydrogen phosphate, guanidino, S-dithiocarbamate, N-dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraarylborate, tetraalkyl borate, tartarate, salicylate, succinate, citrate, ascorbate, saccharirate, amino acid, hydroxamic acid and thiotosylate.

Whenever R₁, R₂ or Rg is hydrogen, its respective carbon atom in a non-substituted carbon atom.

As used herein, the term “alkyl” is a saturated aliphatic hydrocarbon including straight chain and branched chain groups. Preferably, the alkyl group has 1 to 20 carbon atoms. More preferably, it is a medium size alkyl having 1 to 10 carbon atoms. Most preferably, it is a lower alkyl having 1 to 4 carbon atoms. The alkyl group may be substituted or non-substituted. When substituted, the substituent group can be, for example, cycloalkyl, aryl, heteroaryl, heteroalicyclic, hydroxy, alkoxy, aryloxy, thiohydroxy, thioalkoxy, thioaryloxy, cyano, halo, carbonyl, thiocarbonyl, O-carbamate, N-carbamate, O-thiocarbarnate, N-thiocarbamate, C-amido, N-amido, C-carboxy, O-carboxy, nitro, sulfonamide, silyl, guanidine, urea or amino, as these terms are defined hereinbelow.

The term “alkenyl” describes an alkyl group which consists of at least two carbon atoms and at least one carbon-carbon double bond.

The term “alkynyl” describes an alkyl group which consists of at least two carbon atoms and at least one carbon-carbon triple bond.

The term “cycloalkyl” describes an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, aryl, heteroaryl, heteroalicyclic, hydroxy, alkoxy, aryloxy, thiohydroxy, thioalkoxy, thioaryloxy, cyano, halo, carbonyl, thiocarbonyl, C-carboxy, O-carboxy, O-carbarnate, N-carbamate, C-amido, N-amido, nitro, or amino, as these terms are defined hereinabove or hereinbelow.

The term “aryl” describes an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, halo, trihalomethyl, alkyl, hydroxy, alkoxy, aryloxy, thiohydroxy, thiocarbonyl, C-carboxy, O-carboxy, O-carbamate, N-carbamate, O-thiocarbamate, N-thiocarbamate, C-amido, N-amido, sulfinyl, sulfonyl or amino, as these terms are defined hereinabove or hereinbelow.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, cycloalkyl, halo, trihalomethyl, hydroxy, alkoxy, aryloxy, thiohydroxy, thiocarbonyl, sulfonamide, C-carboxy, O-carboxy, sulfinyl, sulfonyl, O-carbamate, N-carbamate, O-thiocarbamate, N-thiocarbamate, C-amido, N-amido or amino, as these terms are defined hereinabove or hereinbelow.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, alkyl, cycloalkyl, aryl, heteroaryl, halo, trihalomethyl, hydroxy, alkoxy, aryloxy, thiohydroxy, thioalkoxy, thioaryloxy, cyano, nitro, carbonyl, thiocarbonyl, C-carboxy, O-carboxy, O-carbamate, N-carbamate, O-thiocarbamate, N-thiocarbamate, sulfinyl, sulfonyl, C-amido, N-amido or amino, as these terms are defined hereinabove or hereinbelow.

The term “halo” describes a fluorine, chlorine, bromine or iodine atom.

The term “amino”, as is defined hereinabove with respect to an “amine” or an “amino group”, is used herein to describe an —NR′R″, wherein R′and R″are each independently hydrogen, alkyl, cycloalkyl, aryl, heteroaryl or heterocyclic, as these terms are defined hereinabove.

Hence, the terms “alkylamino”, “arylamino”, “cycloalkylamino”, “heteroalicyclic amino” and “heteroarylamino” describe an amino group, as defined hereinabove, wherein at least one of R′ and R″ thereof is alkyl, aryl, cycloalkyl, heterocyclic and heteroaryl, respectively.

The term “hydroxy” describes an —OH group.

An “alkoxy” describes both an —O-alkyl and an —O-cycloalkyl group, as defined herein.

An “aryloxy” describes both an —O-aryl and an —O-heteroaryl group, as defined herein.

The term “azo” describes a —N═N group.

A “C-amido” describes a —C(═O)—NR′R″ group, where R′ and R″ are as defined hereinabove.

An “N-amido” describes a R′C(═O)—NR″— group, where R′ and R″ are as defined hereinabove.

An “ammonium” describes an —N⁺HR′R″ group, where R′ and R″ are as defined hereinabove.

The term “thiohydroxy” describes a —SH group.

The term “thioalkoxy” describes both a —S-alkyl group and a 13 S-cycloalkyl group, as defined hereinabove.

The term “thioaryloxy” describes both a —S-aryl and a —S-heteroaryl group, as defined hereinabove.

A “sulfinyl” describes a —S(═O)—R group, where R can be, without limitation, alkyl, cycloalkyl, aryl and heteroaryl as these terms are defined hereinabove.

A “sulfonyl” describes a —S(═O)₂—R group, where R is as defined hereinabove.

A “S-sulfonamido” is a —S(═O)₂—NR′R″ group, with R′ and R″ as defined hereinabove.

A “N-sulfonamido” is an R′(S═O)₂—NR″— group, with R′ and R″ as defined hereinabove.

A “phosphonyl” is a —O—P(═O)(OR′)—R″ group, with R′ and R″ as defined hereinabove.

A “phosphinyl” is a —PR′R″ group, with R′ and R″ as defined hereinabove.

A “phosphonium” is a —P⁺R′R″R′″, where R′ and R″ are as defined hereinabove and R′″ is defined as either R′ or R″.

The term “carbonyl” describes a —C(═O)—R group, where R is hydrogen, alkyl, cycloalkyl, aryl, heteroaryl (bonded through a ring carbon) or heteroalicyclic (bonded through a ring carbon) as defined hereinabove.

A “thiocarbonyl” describes a —C(═S)—R group, where R is as defined hereinabove with respect to the term “carbonyl”.

A “C-carboxy” describes a —C(═O)—O—R groups, where R is as defined hereinabove with respect to the term “carbonyl”.

An “O-carboxy” group refers to a RC(═O)—O— group, where R is as defined hereinabove with respect to the term “carbonyl”.

A “carboxylic acid” is a C-carboxy group in which R is hydrogen.

A “C-thiocarboxy” is a —C(═S)—O—R groups, where R is as defined hereinabove with respect to the term “carbonyl”.

An “O-thiocarboxy” group refers to an R—C(═S)—O— group, where R is as defined hereinabove with respect to the term “carbonyl”.

The term “O-carbamate” describes an —OC(═O)—NR′R″ group, with R′ and R″ as defined hereinabove.

A “N-carbamate” describes a R′—O—C(═O)—NR″— group, with R′ and R″ as defined hereinabove.

An “O-thiocarbamate” describes an —O—C(═S)—NR′R″ group, with R′ and R″ as defined hereinabove.

A “N-thiocarbamate” describes a R′OC(═S)NR″— group, with R′ and R″ as defined hereinabove.

The term “urea” describes a —NR′—C(═O)—NR′R″ group, with R′, R″ and R′″ as defined hereinabove.

The term “thiourea” describes a —NR′—C(═S)—NR′R″ group, with R′, R″ and R′″ as defined hereinabove.

The term “borate” describes an —O—B—(OR)₂ group, with R as defined hereinabove.

The term “borane” describes a —B—R′R″ group, with R′ and R″ as defined hereinabove.

The term “boraza” describes a —B(R′)(NR″R′″) group, with R′, R″ and R′″ as defined hereinabove.

The term “silyl” describes a —SiR′R″R′″, with R′, R″ and R′″ as defined herein.

The term “siloxy” is a —Si—(OR)₃, with R as defined hereinabove.

The term “silaza” describes a —Si—(NR′R″)₃, with R′ and R″ as defined herein.

The term “aquo” describes a H₂O group.

The term “alcohol” describes a ROH group, with R as defined hereinabove.

The term “peroxo” describes an —OOR group, with R as defined hereinabove.

As used herein, an “amine oxide” is a —N(═O)R′R″R′″ group, with R′, R″ and R′″ as defined herein.

A “hydrazine” is a —NR′—NR″R′″ group, with R′, R″ and R′″ as defined herein.

Hence, “alkyl hydrazine” and “aryl hydrazine” describe a hydrazine where R′ is an alkyl or an aryl, respectively, and R″ and R′″ are as defined hereinabove.

The term “nitric oxide” is a —N═O group.

The term “cyano” is a —C≡N group.

A “cyanate” is an —O—C≡N group.

A “thiocyanate” is a “—S—C≡N group.

An “isocyanate” is a —N═C═O group.

An “isothiocyanate” is a —N═C═S group.

The terms “alkyl nitrile” and “aryl nitrile” describe a —R—C≡N group, where R is an alkyl or an aryl, respectively.

The terms “alkyl isonitrile” and “aryl isonitrile” describe a R—N≡C— group, where R is an alkyl or aryl, respectively.

A “nitrate” or “nitro” is a —NO₂ group.

A “nitrite” is an —O—N═O group.

An “azido” is a N₃⁺ group.

An “alkyl sulfonic acid” and an “aryl sulfonic acid” describe a 13 R—SO₂—OH group, with R being an alkyl or an aryl, respectively.

An “alkyl sulfoxide”, an “aryl sulfoxide” and an “alkyl aryl sulfoxide” describe a —R′S(═O)R″ group, where R′ and R″ are each an alkyl, R′ and R″ are each an aryl and where R′ is and alkyl and R″ is an aryl, respectively.

An “alkyl sulfenic acid” and “aryl sulfenic acid” describe a —R—S—OH group, where R is an alkyl or an aryl, respectively.

An “alkyl sulfinic acid” and “aryl sulfinic acid” describe a —R—S(═O)—OH group where R is an alkyl or an aryl, respectively.

As used herein, the terms “alkyl carboxylic acid” and “aryl carboxylic acid” describe a —R—C(═O)—OH group, where R is an alkyl or an aryl, respectively.

An “alkyl thiol carboxylic acid” and an “aryl thiol carboxylic acid” describe a —R—C(═O)—SH group, where R is an alkyl or an aryl, respectively.

An “alkyl thiol thiocarboxylic acid” and an “aryl thiol thiocarboxylic acid” describe a —R—C(═S)—SH group, where R is an alkyl or an aryl, respectively.

A “sulfate” is a —O—SO₂—OR′ group, with R′ as defined hereinabove.

A “sulfite” group is a —O—S(═O)—OR′ group, with R′ as defined hereinabove.

A “bisulfite” is a sulfite group, where R′ is hydrogen.

A “thiosulfate” is an —O—SO₂—SR′ group, with R′ as defined hereinabove.

A “thiosulfite” group is an —O—S(═O)—SR′ group, with R′ as defined hereinabove.

The terms “alkyl/aryl phosphine” describe a —R—PH₂ group, with R being an alkyl or an aryl, respectively, as defined above.

The terms “alkyl and/or aryl phosphine oxide” describe a —R′—PR″₂(═O) group, with R′ and R″ being an alkyl and/or an aryl, as defined hereinabove.

The terms “alkyl and/or aryl phosphine sulfide” describe a —R′—PR″₂(═S) group, with R′ and R″ being an alkyl and/or an aryl, as defined hereinabove.

The terms “alkyl/aryl phosphonic acid” describe a —R′—P(═O)(OH)₂ group, with R′ being an alkyl or an aryl as defined above.

The terms “alkyl/aryl phosphinic acid” describes a —R′—P(OH)₂ group, with R′ being an alkyl or an aryl as defined above.

A “phosphate” is a —O—P(═O)(OR′)(OR″) group, with R′ and R″ as defined hereinabove.

A “hydrogen phosphate” is a phosphate group, where R′ is hydrogen.

A “dihydrogen phosphate” is a phosphate group, where R′ and R″ are both hydrogen.

A “thiophosphate” is a —S—P(═O)(OR′)₂ group, with R′ as defined hereinabove.

A “phosphite” is an —O—P (OR′)₂ group, with R′ as defined hereinabove.

A “pyrophosphite” is an —O—P—(OR′)—O—P(OR″)₂ group, with R′ and R″ as defined hereinabove.

A “triphosphate” describes an —OP(═O)(OR′)—O—P(═O)(OR″)—O—P(═O)(OR′″)₂ with R′, R″ and R′″ are as defined hereinabove.

As used herein, the term “guanidine” describes a —R′NC(═N)—NR″R′″ group, with R′, R″ and R′″ as defined herein.

The term “S-dithiocarbamate” describes a —SC(═S)—NR′R″ group, with R′ and R″ as defined hereinabove.

The term “N-dithiocarbamate” describes an R′SC(═S)—NR″— group, with R′ and R″ as defined hereinabove.

A “bicarbonate” is an —O—C(═O)—O^{- group.}

A “carbonate” is an —O—C(═O)—OH group.

A “perchlorate” is an —O—Cl(═O)₃ group.

A “chlorate” is an —O—Cl(═O)₂ group.

A “chlorite” is an —O—Cl(═O) group.

A “hypochlorite” is an —OCl group.

A “perbromate” is an —O—Br(═O)₃ group.

A “bromate” is an —O—Br(═O)₂ group.

A “bromite” is an —O—Br(═O) group.

A “hypobromite” is an —OBr group.

A “periodate” is an —O—I(═O)₃ group.

A “iodate” is an —O—I(=O)₂ group.

The term “tetrahalomanganate” describes MnCl_{4,} MnBr₄ and MnI_{4.}

The term “tetrafluoroborate” describes a —BF₄ group.

A “tetrafluoroantimonate” is a SbF₆ group.

A “hypophosphite” is a —P(OH)₂ group.

The term “metaborate” describes the group 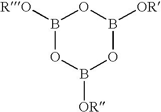
where R′, R″ and R′″ are as defined hereinabove.

The terms “tetraalkyl/tetraaryl borate” describe a R′B— group, with R′ being an alkyl or an aryl, respectively, as defined above.

A “tartarate” is an —OC(═O)—CH(OH)—CH(OH)—C(═O)OH group.

A “salycilate” is the group

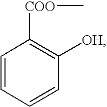

A “succinate” is an —O—C(═O)—(CH₂)₂—COOH group.

A “citrate” is an —O—C(═O)—CH₂—CH(OH)(COOH)—CH₂—COOH group.

An “ascorbate” is the group

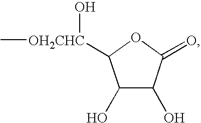

A “saccharirate” is an oxidized saccharide having two carboxylic acid group.

The term “amino acid” as used herein includes natural and modified amino acids and hence includes the 21 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids which are linked via a peptide bond or a peptide bond analog to at least one addition amino acid as this term is defined herein.

A “hydroxamic acid” is a —C(═O)—NH—OH group.

A “thiotosylate” is the group

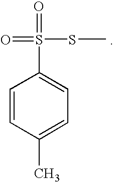

Similarly, each of the alkylene chains B₁ . . . . Bn independently has a general formula III: 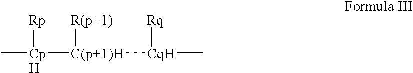
wherein p is an integer that equals 0 or g+1 and q is an integer from g+2 to g+20.

Hence, each of the alkylene chains B₁ . . . . Bn is comprised of a plurality of carbon atoms Cp, Cp+1, Cp+2 . . . . , Cq−1 and Cq, substituted by the respective Rp, Rp+1, Rp+2 . . . . , Rq−1 and Rq groups. Preferably, each of the alkylene chains B₁ . . . . Bn includes 2-20 carbon atoms, more preferably 2-10, and most preferably 2-6 carbon atoms.

As is defined hereinabove, in cases where p equals 0, the component —CpH(Rp)— is absent from the structure. In cases where p equals g+1, it can be either 1 or 4-11. The integer q can be either 2 or 5-20.

Each of the substituents Rp, Rp+1 . . . . Rn can be any of the substituents described hereinabove with respect to R₁, R₂ and Rg.

Hence, a preferred linear polyamine according to the present invention includes two or more alkylene chains. The alkylene chains are interrupted therebetween by a heteroatom and each is connected to a heteroatom at one end thereof. Preferably, each of the alkylene chains include at least two carbon atoms, so as to enable the formation of a stable chelate between the heteroatoms and the copper ion.

The linear polyamine delineated in Formula I preferably includes at least one chiral carbon atom. Hence, at least one of C₁, C₂ and Cg in the alkylene chain A and/or at least one of Cp, Cp+1 and Cq in the alkylene chain B is chiral.

A preferred linear polyamine according to the present invention is tetraethylenepentamine. Other representative examples of preferred linear polyamines usable in the context of the present invention include, without limitation, ethylendiamine, diethylenetriamine, triethylenetetramine, triethylenediamine, aminoethylethanolamine, pentaethylenehexamine, triethylenetetramine, N,N′-bis(3-aminopropyl)-1,3-propanediamine, and N,N′-Bis(2-animoethyl)- 1,3 propanediamine.

In cases where the polyamine chelator is a cyclic polyamine, the polyamine can have a general formula IV: 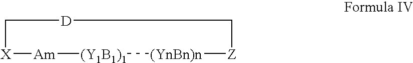
wherein m is an integer from 1 to 10; n is an integer from 0 to 20; X and Z are each independently selected from the group consisting of an oxygen atom, a sulfur atom and a —NH group; Y₁ and Yn are each independently selected from the group consisting of an oxygen atom, a sulfur atom and a —NH group; A is an alkylene chain having between 1 and 10 substituted and/or non-substituted carbon atoms; B₁ and Bn are each independently an alkylene chain having between 1 and 20 substituted and/or non-substituted carbon atoms; and D is a bridging group having a general formula V:
U—W—V Formula V
whereas U and V are each independently selected from the group consisting of substituted hydrocarbon chain and non-substituted hydrocarbon chain; and W is selected from the group consisting of amide, ether, ester, disulfide, thioether, thioester, imine and alkene, provided that at least one of said X, Z, Y₁, and Yn is a —NH group and/or at least one of said carbon atoms in said alkylene chains is substituted by an amine group.

Optionally, the cyclic polyamine has one of the general formulas VI-X: 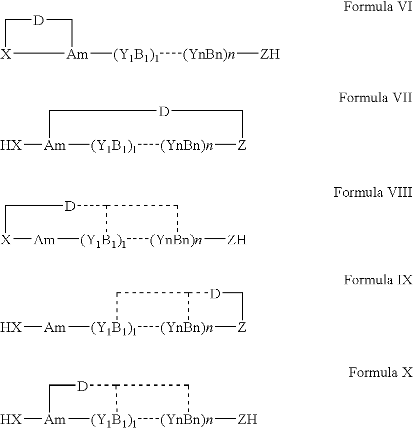
wherein m, n, X, Y₁, Yn, Z, A, B and D are as described above and further wherein should the bridging group D is attached at one end to A (Formulas VI, VII and X), U or V are being attached to one carbon atom in the alkylene chain and should D is attached at one end to B1 or Bn (Formulas VIII, IX and X), U or V are being attached to one carbon atom in the alkylene chain.

Hence, a preferred cyclic polyamine according to the present invention includes two or more alkylene chains, A, B₁ . . . . Bn, as is detailed hereinabove with respect to the linear polyamine. The alkylene chains can form a cyclic structure by being connected, via the bridging group D, between the ends thereof, namely between the heteroatoms X and Z (Formula IV). Optionally, the alkylene chains can form a conformationally restricted cyclic structure by being connected, via the bridging group D, therebetween (Formula X). Further optionally, a conformationally restricted cyclic structure can be formed by connecting one alkylene chain to one terminal heteroatom (X or Z, Formulas VI-IX).

As is described hereinabove, in cases where the cyclic structure is formed by connecting one alkylene chain to one terminal heteroatom, as is depicted in Formulas VI-IX, the bridging group D connects a terminal heteroatom, namely X or Z, and one carbon atom in the alkylene chains A and B₁ . . . . Bn. This carbon atom can be anyone of C₁, C₂, Cg, Cp, Cp+1 and Cq described hereinabove.

As is further described hereinabove, the cyclic structure is formed by the bridging group D, which connects two components in the structure. The bridging group D has a general formula U—W—V, where each of U and V is a substituted or non-substituted hydrocarbon chain.

As used herein, the phrase “hydrocarbon chain” describes a plurality of carbon atoms which are covalently attached one to another and are substituted, inter alia, by hydrogen atoms. The hydrocarbon chain can be saturated, unsaturated, branched or unbranched and can therefore include one or more alkyl, alkenyl, alkynyl, cycloalkyl and aryl groups and combinations thereof.

The length of the hydrocarbon chains, namely the number of carbon atoms in the chains, is preferably determined by the structure of the cyclic polyamine, such that on one hand, the ring tension of the formed cyclic structure would be minimized and on the other hand, an efficient chelation with the copper ion would be achieved.

When the hydrocarbon chain is substituted, the substituents can be any one or combinations of the substituents described hereinabove with respect to R₁, R₂ and Rg in the linear polyamine.

The two hydrocarbon chains are connected therebetween by the group W, which can be amide, ether, ester, disulfide, thioether, thioester, imine and alkene.

As used herein, the term “ether” is an —O— group.

The term “ester” is a —C(═O)—O— group.

A “disulfide” is a —S—S— group.

A “thioether” is a —S— group.

A “thioester” is a —C(═O)—S— group.

An “imine” is a —C(═NH)— group.

An “alkene” is a —CH═CH— group.

The bridging group D is typically formed by connecting reactive derivatives of the hydrocarbon chains U and V, so as to produce a bond therebetween (W), via well-known techniques, as is described, for example, in U.S. Pat. No. 5,811,392.

As is described above with respect to the linear polyamine, the cyclic polyamine must include at least one amine group, preferably at least two amine groups and more preferably at least four amine groups, so as to form a stable copper chelate.

A preferred cyclic polyamine according to the present invention is cyclam (1,4,8,11-tetraazacyclotetradecane).

As is described hereinabove, the polyamine chelator of the present invention can further include a multimeric combination of one or more linear polyamine(s) and one or more cyclic polyamine(s). Such a polyamine chelator can therefore be comprised of any combinations of the linear and cyclic polyamines described hereinabove.

Preferably, such a polyamine chelator has a general Formula XI:
{(E₁)_{f}—[Q₁—(G₁)_{g}]}ₕ—{(E₂)ᵢ—[Q₂—(G₂)ⱼ]}ₖ—. . . . . . . —{(Eₙ)ₗ—[Qₙ—(Gₙ)ₒ]}_{t +L} Formula XI
wherein n is an integer greater than 1; each of f, g, h, i, j, k, l, o and t is independently an integer from 0 to 10; each of E₁, E₂ and En is independently a linear polyamine, as is described hereinabove; each of G_{1,} G₂ and Gn is independently a cyclic polyamine as is described hereinabove; and each of Q₁, Q₂ and Qn is independently a linker linking between two of said polyamines, provided that at least one of said Q₁, Q₂ and Qn is an amine group and/or at least one of said linear polyamine and said cyclic polyamine has at least one free amine group.

Each of E₁, E₂ and En in Formula XI represent a linear polyamine as is described in detail hereinabove, while each of G₁, G₂ and Gn represents a cyclic polyamine as is described in detail hereinabove.

The polyamine described in Formula XI can include one or more linear polyamine(s), each connected to another linear polyamine or to a cyclic polyamine.

Each of the linear or cyclic polyamines in Formula XI is connected to another polyamine via one or more linker(s), represented by Q₁, Q₂ and Qn in Formula XI.

Each of the linker(s) Q₁, Q₂ and Qn can be, for example, alkylene, alkenylene, alkynylene, arylene, cycloalkylene, hetroarylene, amine, azo, amide, sulfonyl, sulfinyl, sulfonamide, phosphonyl, phosphinyl, phosphonium, ketoester, carbonyl, thiocarbonyl, ester, ether, thioether, carbamate, thiocarbamate, urea, thiourea, borate, borane, boroaza, silyl, siloxy and silaza.

As used herein, the term “alkenylene” describes an alkyl group which consists of at least two carbon atoms and at least one carbon-carbon double bond.

The term “alkynylene” describes an alkyl group which consists of at least two carbon atoms and at least one carbon-carbon triple bond.

The term “cycloalkylene” describes an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane.

The term “arylene” describes an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted.

The term “heteroarylene” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted.

As used in the context of the linker of the present invention, the term “amine” describes an —NR′—, wherein R′ can be hydrogen, alkyl, cycloalkyl, aryl, heteroaryl or heterocyclic, as these terms are defined hereinabove.

As is further used in the context of the linker of the present invention, the term “azo” describes a —N═N— group.

The term “amide” describes a —C(═O)—NR′— group, where R′ is as defined hereinabove.

The term “ammonium” describes an —N⁺HR′— group, where R′ is as defined hereinabove.

The term “sulfinyl” describes a ′S(═O)— group.

The term “sulfonyl” describes a —S(═O)₂— group.

The term “sulfonamido” describes a —S(═O)₂—NR′— group, with R′ as defined hereinabove.

The term “phosphonyl” describes a —O—P(═O)(OR′)—group, with R′ as defined hereinabove.

The term “phosphinyl” describes a —PR′— group, with R′ as defined hereinabove.

The term “phosphonium” is a —P⁺R′R″, where R′ and R″ are as defined hereinabove.

The term “ketoester” describes a —C(═O)—C(═O)—O— group.

The term “carbonyl” describes a —C(═O)— group.

The term “thiocarbonyl” describes a —C(═S)— group.

The term “carbamate” describes an —OC(═O)—NR′— group, with R′ as defined hereinabove.

The term “thiocarbamate” describes an —OC(═S)—NR— group, with R′ as defined hereinabove.

The term “urea” describes an —NR′—C(═O)—NR″— group, with R′ and R″ and as defined hereinabove.

The term “thiourea” describes a —NR′—C(═S)—NR′— group, with R′ and R″ as defined hereinabove.

The term “borate” describes an —O—B—(OR)— group, with R as defined hereinabove.

The term “borane” describes a —B—R—′— group, with R as defined hereinabove.

The term “boraza” describes a —B (NR′R″)— group, with R′ and R″ as defined hereinabove.

The term “silyl” describes a —SiR′R″—, with R′ and R″ as defined herein.

The term “siloxy” is a —Si—(OR)₂—, with R as defined hereinabove.

The term “silaza” describes a —Si—(NR′R″)₂—, with R′ and R″ as defined herein.

It should be noted that all the terms described hereinabove in the context of the linker of the present invention are the same as described above with respect to the substituents. However, in distinction from the substituent groups, which are connected to a component at one end thereof, the linker groups are connected to two components at two sites thereof and hence, these terms have been redefined with respect to the linker.

As has been mentioned hereinabove, according to the presently most preferred embodiment of the present invention, the polyamine chelator is tetraethylenepentamine (TEPA). However, other preferred polyamine chelators include, without limitation, ethylendiamine, diethylenetriamine, triethylenetetramine, triethylenediamine, aminoethylethanolamine, aminoethylpiperazine, pentaethylenehexamine, triethylenetetramine, captopril, penicilamine, N,N′-bis(3-aminopropyl)-1,3-propanediamine, N,N′-Bis(2-animoethyl)-1,3-propanediamine, 1,7-dioxa-4,10-diazacyclododecane, 1,4,8,11 -tetraazacyclotetradecane-5,7-dione, 1,4,7-triazacyclononane, 1-oxa-4,7,10-triazacyclododecane, 1,4,8,12-tetraazacyclopentadecane and 1,4,7,10-tetraazacyclododecane.

The above listed preferred chelators are known in their high affinity towards copper ions. However, these chelators are further beneficially characterized by their substantial affinity also towards other transition metals, as is described by Ross and Frant [22], which is incorporated by reference as if fully set forth herein.

All the polyamine chelators described hereinabove can be either commercially obtained or can be synthesized using known procedures such as described, for example, in: T. W. Greene (ed.), 1999 (“Protective Groups in Organic Synthesis” 3ed Edition, John Wiley & Sons, Inc., New York 779 pp); or in: R. C. Larock and V. C. H. Wioley, “Comprehensive Organic Transformations—A Guide to Functional Group Preparations”, (1999) 2^{nd} Edition.

The copper chelate can be provided to the cell culture medium. The final concentrations of copper chelate may be, depending on the specific application, in the micromolar or millimolar ranges, for example, within about 0.1 μM to about 100 μM, preferably within about 4 μM to about 50 mM, more preferably within about 5 μM to about 40 μM. As is described hereinabove, the copper chelate is provided to the cells so as to maintain the free copper concentration of the cells substantially unchanged during cell expansion.

The stem and/or progenitor cells used in the present invention can be of various origin. According to a preferred embodiment of the present invention, the stem and/or progenitor cells are derived from a source selected from the group consisting of hematopoietic cells, umbilical cord blood cells, G-CSF mobilized peripheral blood cells, bone marrow cells, hepatic cells, pancreatic cells, neural cells, oligodendrocyte cells, skin cells, embryonal stem cells, muscle cells, bone cells, mesenchymal cells, chondrocytes and stroma cells. Methods of preparation of stem cells from a variety of sources are well known in the art, commonly selecting cells expressing one or more stem cell markers such as CD34, CD133, etc, or lacking markers of differentiated cells. Selection is usually by FACS, or immunomagnetic separation, but can also be by nucleic acid methods such as PCR (see Materials and Experimental Methods hereinbelow). Embryonic stem cells and methods of their retrieval are well known in the art and are described, for example, in Trounson A O (Reprod Fertil Dev (2001) 13: 523), Roach M L (Methods Mol Biol (2002) 185: 1), and Smith A G (Annu Rev Cell Dev Biol (2001) 17:435). Adult stem cells are stem cells, which are derived from tissues of adults and are also well known in the art. Methods of isolating or enriching for adult stem cells are described in, for example, Miraglia, S. et al. (1997) Blood 90: 5013, Uchida, N. et al. (2000) Proc. Natl. Acad. Sci. USA 97: 14720, Simmons, P. J. et al. (1991) Blood 78: 55, Prockop D J (Cytotherapy (2001) 3: 393), Bohmer R M (Fetal Diagn Ther (2002) 17: 83) and Rowley S D et al. (Bone Marrow Transplant (1998) 21: 1253), Stem Cell Biology Daniel R. Marshak (Editor) Richard L. Gardner (Editor), Publisher: Cold Spring Harbor Laboratory Press, (2001) and Hematopoietic Stem Cell Transplantation. Anthony D. Ho (Editor) Richard Champlin (Editor), Publisher: Marcel Dekker (2000).

Ianus et al. (J Clin. Invest 2003;1 11:843-850) demonstrated that nucleated bone marrow cells from GFP-transgenic mice, when implanted into wild type mice, produced pancreatic islet cells expressing GFP. However, the bone marrow cell fraction implanted was not expanded ex-vivo prior to implantation. PCT IL03/00681 to Peled, et al, which is incorporated by reference as if fully set for herein, discloses the use of molecules such as copper chelators, copper chelates and retinoic acid receptor (RAR) antagonists which are capable of repressing differentiation and stimulating and prolonging proliferation of hematopoietic stem cells when the source of cells includes the entire fraction of mononuclear blood cells, namely non-enriched stem cells. Thus, in one embodiment of the present invention, the population of cells comprising stem and/or progenitor cells is unselected mononuclear cells.

As used herein, the phrase “hematopoietic mononuclear cells” refers to the entire repertoire of white blood cells present in a blood sample, usually hematopoietic mononuclear cells which comprise a major fraction of hematopoietic committed cells and a minor fraction of hematopoietic stem and progenitor cells. In a healthy human being, the white blood cells comprise a mixture of hematopoietic lineages committed and differentiated cells (typically over 99% of the mononuclear cells are lineages committed cells) including, for example: Lineage committed progenitor cells CD34⁺CD33⁺ (myeloid committed cells), CD34⁺CD3⁺ (lymphoid committed cells) CD34⁺CD41⁺ (megakaryocytic committed cells) and differentiated cells −CD34⁻CD33⁺ (myeloids, such as granulocytes and monocytes), CD34⁻CD3⁺, CD34^{-CD19}⁺ (T and B cells, respectively), CD34^{-CD41}⁺ (megakaryocytes), and hematopoietic stem and early progenitor cells such as CD34⁺ Lineage negative (Lin), CD34 −Lineage negative CD34⁺CD38⁻ (typically less than 1%).

The phrase “hematopoietic mononuclear cells which comprise a major fraction of hematopoietic committed cells and a minor fraction of hematopoietic stem and progenitor cells” is used herein to describe any portion of the white blood cells fraction, in which the majority of the cells are hematopoietic committed cells, while the minority of the cells are hematopoietic stem and progenitor cells, as these terms are further defined hereinunder.

Hematopoietic mononuclear cells are typically obtained from a blood sample by applying the blood sample onto a Ficoll-Hypaque layer and collecting, following density-cushion centrifugation, the interface layer present between the Ficoll-Hypaque and the blood serum, which interface layer essentially entirely consists of the white blood cells present in the blood sample.

Presently, hematopoietic stem cells are obtained by further enrichment of the hematopoietic mononuclear cells obtained by differential density centrifugation as described above. This further enrichment process is typically performed by immuno-separation such as immunomagnetic-separation or FACS and results in a cell fraction that is enriched for hematopoietic stem cells (for detailed description of enrichment of hematopoietic stem cells, see Materials and Experimental Procedures in the Examples section hereinbelow).

Hence, using hematopoietic mononuclear cells as a direct source for obtaining expanded population of hematopoietic stem cells circumvents the need for stem cell enrichment prior to expansion, thereby substantially simplifying the process in terms of both efficiency and cost.

According to one aspect of the present invention, there is provided a conditioned medium isolated from expanded stem and/or progenitor cells cultured according to the methods of the present invention in a bioreactor. Such cultured medium can comprise growth factors, cytokines, cellular metabolites and secreted biomolecules useful in controlling/enhancing growth in subsequent cultures of stem, progenitor or cells at various stages of differentiation, from diverse sources. Further, such biologically active cultured media could eventually provide valuable clues to the processes of differentiation.

Thus, according to a further aspect of the present invention, there is provided a method of preparing a stem and/or progenitor cell conditioned medium, the method comprising (a) establishing a stem and/or progenitor cells culture in a bioreactor, as described in detail hereinabove, thereby expanding the stem and/or progenitor cells while at the same time, substantially inhibiting differentiation of the cells, and (b) when a desired stem and/or progenitor cell density is achieved, collecting medium from the bioreactor, thereby obtaining the stem and/or progenitor cell conditioned medium. It will be appreciated that whereas the conditioned medium can be collected from any of the abovementioned bioreactors, the perfused bioreactors such as continuous, direct perfusion, perfused spinner flask bioreactors, and perfuse rotating wall vessel bioreactors are most suitable for collection of conditioned medium, directly from the medium effluent channels. A bioreactor support system disclosed by Gruenberg (PCT Publication No. WO03025158), which makes use of a cell separator module in advance of the medium conditioning stage (oxygenation, nutrition, waste removal, etc), is particularly suited for production of stem and/or progenitor cells conditioned medium.

Determination of the desired cell density within the bioreactor suitable for collection of medium will depend upon the intended use of the conditioned medium. Using standard bioassays, such as proliferation and differentiation assays (specific CD clusters, for example), one of ordinary skill in the art can determine the appropriate bioreactor cell density for preparation of conditioned medium. Similarly, if specific factor or metabolite is desired, the medium can be monitored and removed at the point of greatest concentration.

According to another aspect of the present invention, the ex-vivo expansion of populations of stem cells in a bioreactor, according to the features described hereinabove, can be utilized for expanding a population of renewable stem and/or progenitor cells ex-vivo for transplanting the cells in a recipient.

Transplanting can be by means of direct injection into a specific organ, injection into the bloodstream, intraperitoneal injection, etc. Suitable methods of transplantation can be determined by monitoring the homing of the implanted cells to a desired organ, the expression of desired organ-specific genes or markers, and the function of the organ in the recipient. Methods of cellular therapy, that is, transplanting stem and/or progenitor cells into a recipient are well know in the art (see, for example, the numerous references in the Background section hereinabove). Reisner et al. (U.S. Pat. No. 5,806,529, which is incorporated by reference as if fully set forth by reference herein) teach the transplantation of stem cells and bone marrow cells to cancer patients, following bone marrow ablation. Reisner et al. also teach methods for recipient conditioning, such as immunosuppression, to prevent and/or suppress rejection of the transplanted cells. Such rejection is the greatest obstacle to the successful engraftment of transplanted cells. Slavin (U.S. Pat. No. 6,143,292, which is incorporated by reference as if fully set forth by reference herein) also describes methods of cell transplantation, specifically allogeneic lymphocyte transplantation, for eradication of remnant host tumor cells following bone marrow transplant in cancer patients. For a comprehensive treatment of the subject of cell transplantation, see Prockoll, et al., PNAS 2003, 100: 11917-923.

As described hereinabove, and detailed in the Examples section hereinbelow, prior to implantation the stem and/or progenitor cells are cultured ex-vivo under conditions allowing for cell proliferation and, at the same time, substantially inhibiting differentiation thereof. According to preferred embodiments of the present invention, providing the stem cells with the conditions for ex-vivo cell proliferation comprises providing the cells with nutrients and with cytokines. Preferably, the cytokines are early acting cytokines, such as, but not limited to, stem cell factor, FLT3 ligand, interleukin-1, interleukin-2, interleukin-3, interleukin-6, interleukin-10, interleukin-12, tumor necrosis factor-α and thrombopoietin. It will be appreciated in this respect that novel cytokines are continuously discovered, some of which may find uses in the methods of cell expansion of the present invention.

Late acting cytokines can also be used. These include, for example, granulocyte colony stimulating factor, granulocyte/macrophage colony stimulating factor, erythropoietin, FGF, EGF, NGF, VEGF, LIF, Hepatocyte growth factor and macrophage colony stimulating factor.

The present invention can be used for gene therapy. Gene therapy as used herein refers to the transfer of genetic material (e.g., DNA or RNA) of interest into a host to treat or prevent a genetic or acquired disease or condition or phenotype. The genetic material of interest encodes a product (e.g., a protein, polypeptide, peptide, functional RNA, antisense) whose production in vivo is desired. For example, the genetic material of interest can encode a hormone, receptor, enzyme, polypeptide or peptide of therapeutic value. For review see, in general, the text “Gene Therapy” (Advanced in Pharmacology 40, Academic Press, 1997).

Two basic approaches to gene therapy have evolved: (i) ex-vivo or cellular gene therapy; and (ii) in vivo gene therapy. In ex-vivo gene therapy cells are removed from a patient, and while being cultured are treated in-vitro. Generally, a functional replacement gene is introduced into the cells via an appropriate gene delivery vehicle/method (transfection, transduction, homologous recombination, etc.) and an expression system as needed and then the modified cells are expanded in culture and returned to the host/patient. These genetically re-implanted cells have been shown to express the transfected genetic material in situ.

Hence, in one embodiment of the present invention, the stem and/or progenitor cells are genetically modified cells. In a preferred embodiment, genetically modifying the cells is effected by a vector, which comprises the exogene or transgene, which vector is, for example, a viral vector or a nucleic acid vector. Many viral vectors suitable for use in cellular gene therapy are known, examples are provided hereinbelow. Similarly, a range of nucleic acid vectors can be used to genetically transform the expanded cells of the invention, as is further described below.

Accordingly, the expanded cells of the present invention can be modified to express a gene product. As used herein, the phrase “gene product” refers to proteins, peptides and functional RNA molecules. Generally, the gene product encoded by the nucleic acid molecule is the desired gene product to be supplied to a subject. Examples of such gene products include proteins, peptides, glycoproteins and lipoproteins normally produced by an organ of the recipient subject. For example, gene products which may be supplied by way of gene replacement to defective organs in the pancreas include insulin, amylase, protease, lipase, trypsinogen, chymotrypsinogen, carboxypeptidase, ribonuclease, deoxyribonuclease, triaclyglycerol lipase, phospholipase A₂, elastase, and amylase; gene products normally produced by the liver include blood clotting factors such as blood clotting Factor VIII and Factor IX, UDP glucuronyl transferae, ornithine transcarbanoylase, and cytochrome p450 enzymes, and adenosine deaminase, for the processing of serum adenosine or the endocytosis of low density lipoproteins; gene products produced by the thymus include serum thymic factor, thymic humoral factor, thymopoietin, and thymosin α₁; gene products produced by the digestive tract cells include gastrin, secretin, cholecystokinin, somatostatin, serotinin, and substance P.

Alternatively, the encoded gene product is one, which induces the expression of the desired gene product by the cell (e.g., the introduced genetic material encodes a transcription factor, which induces the transcription of the gene product to be supplied to the subject).

In still another embodiment, the recombinant gene can provide a heterologous protein, e.g., not native to the cell in which it is expressed. For instance, various human MHC components can be provided to non-human cells to support engraftment in a human recipient. Alternatively, the transgene is one, which inhibits the expression or action of a donor MHC gene product.

A nucleic acid molecule introduced into a cell is in a form suitable for expression in the cell of the gene product encoded by the nucleic acid. Accordingly, the nucleic acid molecule includes coding and regulatory sequences required for transcription of a gene (or portion thereof) and, when the gene product is a protein or peptide, translation of the gene acid molecule include promoters, enhancers and polyadenylation signals, as well as sequences necessary for transport of an encoded protein or peptide, for example N-terminal signal sequences for transport of proteins or peptides to the surface of the cell or secretion.

Nucleotide sequences which regulate expression of a gene product (e.g., promoter and enhancer sequences) are selected based upon the type of cell in which the gene product is to be expressed and the desired level of expression of the gene product. For example, a promoter known to confer cell-type specific expression of a gene linked to the promoter can be used. A promoter specific for myoblast gene expression can be linked to a gene of interest to confer muscle-specific expression of that gene product. Muscle-specific regulatory elements, which are known in the art, include upstream regions from the dystrophin gene (Klamut et al., (1989) Mol. Cell Biol. 9: 2396), the creatine kinase gene (Buskin and Hauschka, (1989) Mol. Cell Biol. 9: 2627) and the troponin gene (Mar and Ordahl, (1988) Proc. Natl. Acad. Sci. USA. 85: 6404). Regulatory elements specific for other cell types are known in the art (e.g., the albumin enhancer for liver-specific expression; insulin regulatory elements for pancreatic islet cell-specific expression; various neural cell-specific regulatory elements, including neural dystrophin, neural enolase and A4 amyloid promoters).

Alternatively, a regulatory element, which can direct constitutive expression of a gene in a variety of different cell types, such as a viral regulatory element, can be used. Examples of viral promoters commonly used to drive gene expression include those derived from polyoma virus, Adenovirus 2, cytomegalovirus and Simian Virus 40, and retroviral LTRs.

Alternatively, a regulatory element, which provides inducible expression of a gene linked thereto, can be used. The use of an inducible regulatory element (e.g., an inducible promoter) allows for modulation of the production of the gene product in the cell. Examples of potentially useful inducible regulatory systems for use in eukaryotic cells include hormone-regulated elements (e.g., see Mader, S. and White, J. H. (1993) Proc. Natl. Acad. Sci. USA 90: 5603-5607), synthetic ligand-regulated elements (see, e.g., Spencer, D. M. et al. 1993) Science 262: 1019-1024) and ionizing radiation-regulated elements (e.g., see Manome, Y. Et al. (1993) Biochemistry 32: 10607-10613; Datta, R. et al. (1992) Proc. Natl. Acad. Sci. USA 89: 1014-10153). Additional tissue-specific or inducible regulatory systems, which may be developed, can also be used in accordance with the invention.

There are a number of techniques known in the art for introducing genetic material into a cell that can be applied to modify a cell of the invention.

In one embodiment, the nucleic acid is in the form of a naked nucleic acid molecule. In this situation, the nucleic acid molecule introduced into a cell to be modified consists only of the nucleic acid encoding the gene product and the necessary regulatory elements.

Alternatively, the nucleic acid encoding the gene product (including the necessary regulatory elements) is contained within a plasmid vector. Examples of plasmid expression vectors include CDM8 (Seed, B. (1987) Nature 329: 840) and pMT2PC (Kaufman, et al. (1987) EMBO J. 6: 187-195).

In another embodiment, the nucleic acid molecule to be introduced into a cell is contained within a viral vector. In this situation, the nucleic acid encoding the gene product is inserted into the viral genome (or partial viral genome). The regulatory elements directing the expression of the gene product can be included with the nucleic acid inserted into the viral genome (i.e., linked to the gene inserted into the viral genome) or can be provided by the viral genome itself.

Naked nucleic acids can be introduced into cells using calcium phosphate mediated transfection, DEAE-dextran mediated transfection, electroporation, liposome-mediated transfection, direct injection, and receptor-mediated uptake.

Naked nucleic acid, e.g., DNA, can be introduced into cells by forming a precipitate containing the nucleic acid and calcium phosphate. For example, a HEPES-buffered saline solution can be mixed with a solution containing calcium chloride and nucleic acid to form a precipitate and the precipitate is then incubated with cells. A glycerol or dimethyl sulfoxide shock step can be added to increase the amount of nucleic acid taken up by certain cells. CaPO₄-mediated transfection can be used to stably (or transiently) transfect cells and is only applicable to in vitro modification of cells. Protocols for CaPO₄-mediated transfection can be found in Current Protocols in Molecular Biology, Ausubel, F. M. et al. (eds.) Greene Publishing Associates, (1989), Section 9.1 and in Molecular Cloning: A Laboratory Manual, 2nd Edition, Sambrook et al. Cold Spring Harbor Laboratory Press, (1989), Sections 16.32-16.40 or other standard laboratory manuals.

Naked nucleic acid can be introduced into cells by forming a mixture of the nucleic acid and DEAE-dextran and incubating the mixture with the cells. A dimethylsulfoxide or chloroquine shock step can be added to increase the amount of nucleic acid uptake. DEAE-dextran transfection is only applicable to in vitro modification of cells and can be used to introduce DNA transiently into cells but is not preferred for creating stably transfected cells. Thus, this method can be used for short-term production of a gene product but is not a method of choice for long-term production of a gene product. Protocols for DEAE-dextran-mediated transfection can be found in Current Protocols in Molecular Biology, Ausubel, F. M. et al. (eds.) Greene Publishing Associates (1989), Section 9.2 and in Molecular Cloning: A Laboratory Manual, 2nd Edition, Sambrook et al. Cold Spring Harbor Laboratory Press, (1989), Sections 16.41-16.46 or other standard laboratory manuals.

Naked nucleic acid can also be introduced into cells by incubating the cells and the nucleic acid together in an appropriate buffer and subjecting the cells to a high-voltage electric pulse. The efficiency with which nucleic acid is introduced into cells by electroporation is influenced by the strength of the applied field, the length of the electric pulse, the temperature, the conformation and concentration of the DNA and the ionic composition of the media. Electroporation can be used to stably (or transiently) transfect a wide variety of cell types and is only applicable to in vitro modification of cells. Protocols for electroporating cells can be found in Current Protocols in Molecular Biology, Ausubel F. M. et al. (eds.) Greene Publishing Associates, (1989), Section 9.3 and in Molecular Cloning: A Laboratory Manual, 2nd Edition, Sambrook et al. Cold Spring Harbor Laboratory Press, (1989), Sections 16.54-16.55 or other standard laboratory manuals.

Another method by which naked nucleic acid can be introduced into cells includes liposome-mediated transfection (lipofection). The nucleic acid is mixed with a liposome suspension containing cationic lipids. The DNA/liposome complex is then incubated with cells. Liposome mediated transfection can be used to stably (or transiently) transfect cells in culture in vitro. Protocols can be found in Current Protocols in Molecular Biology, Ausubel F. M. et al. (eds.) Greene Publishing Associates, (1989), Section 9.4 and other standard laboratory manuals. Additionally, gene delivery in vivo has been accomplished using liposomes. See for example Nicolau et al. (1987) Meth. Enz. 149:157-176; Wang and Huang (1987) Proc. Natl. Acad. Sci. USA 84:7851-7855; Brigham et al. (1989) Am. J Med. Sci. 298:278; and Gould-Fogerite et al. (1989) Gene 84:429-438.

Naked nucleic acid can also be introduced into cells by directly injecting the nucleic acid into the cells. For an in vitro culture of cells, DNA can be introduced by microinjection. Since each cell is microinjected individually, this approach is very labor intensive when modifying large numbers of cells. However, a situation wherein microinjection is a method of choice is in the production of transgenic animals (discussed in greater detail below). In this situation, the DNA is stably introduced into a fertilized oocyte, which is then allowed to develop into an animal. The resultant animal contains cells carrying the DNA introduced into the oocyte. Direct injection has also been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo can be used. Such an apparatus is commercially available (e.g., from BioRad).

Naked nucleic acid can be complexed to a cation, such as polylysine, which is coupled to a ligand for a cell-surface receptor to be taken up by receptor-mediated endocytosis (see for example Wu, G. and Wu, C. H. (1988) J BioL Chem. 263: 14621; Wilson et al. (1992) J Biol. Chem. 267: 963-967; and U.S. Pat. No. 5,166,320). Binding of the nucleic acid-ligand complex to the receptor facilitates uptake of the DNA by receptor-mediated endocytosis. Receptors to which a DNA-ligand complex has targeted include the transferrin receptor and the asialoglycoprotein receptor. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88: 8850; Cristiano et al. (1993) Proc. Natl. Acad. Sci. USA 90: 2122-2126). Receptor-mediated DNA uptake can be used to introduce DNA into cells either in vitro or in vivo and, additionally, has the added feature that DNA can be selectively targeted to a particular cell type by use of a ligand which binds to a receptor selectively expressed on a target cell of interest.

Generally, when naked DNA is introduced into cells in culture (e.g., by one of the transfection techniques described above) only a small fraction of cells (about 1 out of 10⁵) typically integrate the transfected DNA into their genomes (i.e., the DNA is maintained in the cell episomally). Thus, in order to identify cells, which have taken up exogenous DNA, it is advantageous to transfect nucleic acid encoding a selectable marker into the cell along with the nucleic acid(s) of interest. Preferred selectable markers include those, which confer resistance to drugs such as G418, hygromycin and methotrexate. Selectable markers may be introduced on the same plasmid as the gene(s) of interest or may be introduced on a separate plasmid.

A preferred approach for introducing nucleic acid encoding a gene product into a cell is by use of a viral vector containing nucleic acid, e.g., a cDNA, encoding the gene product. Infection of cells with a viral vector has the advantage that a large proportion of cells receive the nucleic acid which can obviate the need for selection of cells which have received the nucleic acid. Additionally, molecules encoded within the viral vector, e.g., a cDNA contained in the viral vector, are expressed efficiently in cells which have taken up viral vector nucleic acid and viral vector systems can be used either in vitro or in vivo.

Defective retroviruses are well characterized for use in gene transfer for gene therapy purposes (for review see Miller, A.D. (1990) Blood 76: 271). A recombinant retrovirus can be constructed having a nucleic acid encoding a gene product of interest inserted into the retroviral genome. Additionally, portions of the retroviral genome can be removed to render the retrovirus replication defective. The replication defective retrovirus is then packaged into virions, which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM, which are well known to those skilled in the art. Examples of suitable packaging virus lines include ψCrip, ψCrip, ψ2 and ψAm. Retroviruses have been used to introduce a variety of genes into many different cell types, including epithelial cells endothelial cells, lymphocytes, myoblasts, hepatocytes, bone marrow cells, in vitro and/or in vivo (see for example Eglitis, et al. (1985) Science 230: 1395-1398; Danosand Mulligan (1988) Proc. Natl. Acad. Sci. USA 85: 6460-6464; Wilson et al. (1988) Proc. NatL Acad. Sci USA 85:3014-3018; Armentano et al., (1990) Proc. Natl. Acad. Sci. USA 87: 6141-6145; Huber et al. (1991) Proc. Natl. Acad. Sci. USA 88: 8039-8043; Feri et al. (1991) Proc. Natl. Acad. Sci. USA 88:8377-8381; Chowdhury et al. (1991) Science 254: 1802-1805; van Beusechem et al. (1992) Proc. Natl. Acad. Sci USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89: 6482-6486), hepatocytes (Herz and Gerard (1993) Proc. Natl. Acad. Sci. USA 90: 2812-2816) and muscle cells (Quantin et al. (1992) Proc. Natl. Acad. Sci. USA 89: 2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol 57: 267). Most replication-defective adenoviral vectors currently in use are deleted for all or parts of the viral E1 and E3 genes but retain as much as 80% of the adenoviral genetic material.

Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics In Micro. And Immunol. (1992) 158: 97-129). It is also one of the few viruses that may integrate its DNA into ion-dividing cells, and exhibits a high frequency of stable integration (see for example Flotte et al. (1992) Am. J. Respir. Cell. Mol. Biol. 7: 349-356; Samulski et al. (1989) J. Virol. 63:3822-3828; and McLaughlin et al. (1989) J. Virol. 62: 1963-1973). Vectors containing as little as 300 base pairs of AAV can be packaged and can integrate. Space for exogenous DNA is limited to about 4.5 kb. An AAV vector such as that described in Tratschin et al. (1 985) Mol. Cell. Biol. 5: 3251-3260 can be used to introduce DNA into cells. A variety of nucleic acids have been introduced into different cell types using AAV vectors (see for example Hermonat et al. (1984) Proc. Natl. Acad. Sci. USA 81: 6466-6470; Tratschin et al. (1985) Mol. Cell Biol. 4: 2072-2081; Wondisford et al. (1988) Mol. Endocrinol. 2:32-39; Tratschin et al. (1984) J. Virol. 51: 611-619; and Flotte et al. (1993) J. Biol. Chem. 268: 3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay. If the gene product of interest to be expressed by a cell is not readily assayable, an expression system can first be optimized using a reporter gene linked to the regulatory elements and vector to be used. The reporter gene encodes a gene product, which is easily detectable and, thus, can be used to evaluate efficacy of the system. Standard reporter genes used in the art include genes encoding β-galactosidase, chloramphenicol acetyl transferase, luciferase and human growth hormone.

When the method used to introduce nucleic acid into a population of cells results in modification of a large proportion of the cells and efficient expression of the gene product by the cells (e.g., as is often the case when using a viral expression vector), the modified population of cells may be used without further isolation or subcloning of individual cells within the population. That is, there may be sufficient production of the gene product by the population of cells such that no further cell isolation is needed. Alternatively, it may be desirable to grow a homogenous population of identically modified cells from a single modified cell to isolate cells, which efficiently express the gene product. Such a population of uniform cells can be prepared by isolating a single modified cell by limiting dilution cloning followed by expanding the single cell in culture into a clonal population of cells by standard techniques.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following.

### EXAMPLES

Reference is now made to the following examples, which together with the above descriptions illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization - A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### MATERIALS AND EXPERIMENTAL METHODS

### Cells and Cell Processing for Expansion and Transplantation

Cell Source:

Hematopoietic cells were either hematopoietic stem cells (HSC) or progenitor cells (HPC) from either bone marrow (BM), G-CSF mobilized peripheral blood (MPB) or umbilical cord blood (UCB).

Mesenchymal cells were human mesenchymal stem cells (hMSC) from either bone marrow (BM), G-CSF mobilized peripheral blood (MPB) or umbilical cord blood (UCB).

Endothelial cells were Endothelial Progenitor Cells (EPC, (Rafii et al. 2003)) from either bone marrow (BM), G-CSF mobilized peripheral blood (MPB) or umbilical cord blood (UCB).

Cell Cultures of Human Hematopoietic Stem/Progenitor Cells: Human umbilical cord blood cells were obtained from umbilical cord blood after normal full-term delivery (informed consent was given). MPB, or BM were obtained from donations (informed consent was given). Samples were either used fresh or collected and frozen according to well known cord blood cryopreservation protocol (Rubinstein et al. 1995) within 24 h postpartum for UCB or according to common practice regarding MPB and BM. Prior to cryopreservation, blood was sedimented for 30 minutes on HESPAN Starch (hydroxyethyl starch) to remove most RBC. Prior to their use, the cells were thawed in Dextran buffer (Sigma, St. Louis, Mo., USA) containing 2.5% human serum albumin (HSA) (Bayer Corp. Elkhart, Ind., USA) and processed as described herein below. Following thawing, where indicated, the leukocyte-rich fraction was harvested and layered on Ficoll-Hypaque gradient (1.077 g/mL; Sigma Inc, St Louis Mo., USA), and centrifuged at 400X g for 30 minutes. The mononuclear cell fraction in the interface layer was then collected, washed three times, and re-suspended in phosphate-buffered saline (PBS) (Biological Industries, Bet HaEmek, Israel) containing 0.5% human serum albumin (HSA) (Bayer Corp. Elkhart, Ind., USA). The CD133⁺ cell fraction was purified as follows: Either the mononuclear cell fraction was subjected to two cycles of immuno-magnetic separation using the “MiniMACS CD133 stem cell isolation kit” (Miltenyi Biotec, Auburn, Calif.) or the unfractionated preparation was isolated on the CliniMACS device using CD133⁺ CliniMACS (Miltenyi Biotec, Auburn, Calif.) reagent, accordingly, following the manufacturer's recommendations (in the latter, the Ficoll-Hypaque gradient stage was omitted). The purity of the CD133⁺ population thus obtained was 80-95%, as evaluated by flow cytometry.

Ex Vivo Expansion of CD133⁺ in HSC Conditions: Purified CD133⁺ cells were cultured in culture bags (American Fluoroseal Co. Gaithersburg, Md., USA) at a concentration of 1×10⁴ cells/ml in alpha minimal essential medium (MEMα) supplemented with 10% FCS containing the following human recombinant cytokines: Thrombopoietin (TPO), interleukin-6 (IL-6), FLT-3 ligand and stem cell factor (SCF), each at a final concentration of 50-150 ng/ml (Perpo Tech, Inc., Rocky Hill, N.J., USA), with 5 μM tetraethylenepentamine (TEPA) (Aldrich, Milwaukee, Wis., USA) and incubated at 37° C. in a humidified atmosphere of 5% CO₂ in air. The cultures were topped up weekly with the same volume of fresh medium, TEPA and growth factors during up to three weeks of expansion.

Mesenchymal Stem Cells Isolation and Culture: Mesenchymal stem cell (MSC) cultures were prepared as previous described (Pittenger et al. 1999). Cells that were either collected from surgical aspirates of bone marrow, UCB or PB to prepare ex vivo culture or CD133 + purified cells (see before) were plated at low-density (1.5×10⁴ cells/cm²) and cultured in growth medium containing Dulbecco's Modified Essential Medium (DMEM) with the addition of 10% heat-inactivated fetal calf serum (FCS) (Biological industries, Bet-Haemek, Israel). To generate large number of cells from the primary cultures, the cells were trypsinized and single cell suspensions were re-cultured for 7 days and grown up to 80% confluence and incubated at 37° C. humidified atmosphere with 5% CO₂ for 3 days before the first medium change. The mesenchymal population is isolated based on its ability to adhere to the culture plate (Wakitani et al. 1995; Pereira et al. 1998; Sakai et al. 1999). Following the first medium change, subsequent changes were carried on twice a week. At 90% confluence, the cells were trypsinized (0.25% Trypsin-EDTA, Sigma-Aldrich, St Louis, Mo.) and passaged to 225 cm² flasks at 1:3 ratios. These first passage MSCs are used in all experiments.

In order to assess the percentage of MSCs of the total cells to be used, the polyclonal antibody to the MSCs surface antigen SB-10 (ALCAM) (Santa Cruz Biotechnology, (Bruder et al. 1998)) was used.

Preparation of Endothelial Progenitor Cells: BM, MPB and UCB derived endothelial progenitor cells (EPCs) are prepared as described elsewhere with some modifications (Kawamoto et al. 2003). Either CD133+ or CD31 (+) cells were separated using a Miltenyi Biotec's magnetic cell separation technology (MACS) and suspended in X vivo-15 medium (Biowhittaker, Cambrex BioScience, Verviers, Belgium) supplemented with 1 ng/mL carrier-free human recombinant VEGF (R&D), 0.1 μmol/L atorvastatin (Pfizer Inc, NY, N.Y.), and 20% human serum (Baxter Healthcare, Deerfield, Ill.). Cells were seeded at a density of 6.4×10⁵ cells/mm² at fibronectin-coated dishes (Hoffman LaRoche Ltd., Basel, Switzerland). After 3 days of cultivation, cells were detached with 0.5 mmol/L EDTA, washed twice and resuspended in a final volume of 10 mL X vivo-10 medium. The resulting cell suspension contains a heterogeneous population of progenitor cells.

Ex Vivo Expansion of CD133⁺ Cells in HSC Conditions: Purified CD133⁺ cells were cultured in culture bags (American Fluoroseal Co. Gaithersburg, Md., USA) at a concentration of 2−100×10³ cells/ml in alpha minimal essential medium (MEMα) supplemented with 10% FCS containing the following human recombinant cytokines: Thrombopoietin (TPO), interleukin-6 (IL-6), FLT-3 ligand and stem cell factor (SCF), each at a final concentration of 50-150 ng/ml (Perpo Tech, Inc., Rocky Hill, N.J., USA), with 5 μM tetraethylenepentamine (TEPA) (Aldrich, Milwaukee, Wis., USA) and incubated at 37° C. in a humidified atmosphere of 5% CO₂ in air. The cultures were topped up weekly with the same volume of fresh medium, TEPA and growth factors during up to thirteen weeks of expansion.

Ex Vivo Expansion Under Human Mesenchymal Stem Cell (hMSC-positive) Conditions: Either purified CD133⁺ cells or cells known to be MSC were cultured at concentration of 2−100×10³ cells/ml in either 250 ml tissue culture flasks (T-flask 250) covered with fibronectin and laminin or in tissue culture Teflon bags. The medium contained MEMα with 15% FCS, 2 mM L-glutamine, 25 mM HEPES, 100 μL antibiotics (pen/strep), 1 mM 2-mercaptoethanol and 0.5 μM dexamethasone containing the following human recombinant growth/differentiation factors: bFGF, FGF-1 and FGF-2 (each at 20 ng/ml), LIF, HGF, interleukin-6 (IL-6), OSM, Bone Morphogenetic Protein 6 and 4 (BMP6, BMP4) and stem cell factor (SCF), each at a final concentration of 10-50 ng/ml with 2-15 μM tetraethylenepentamine (TEPA) (Aldrich, Milwaukee, Wis., USA) and incubated at 37° C. in a humidified atmosphere of 5% CO₂ in air. The cultures were topped up weekly with the same volume of fresh medium, TEPA and growth factors up to thirteen weeks of expansion. For the bioreactor experiments the MSC were immobilized on mirocarrier beads (made of Dextran, PGA, Fibrin or Calcium Alginate), as described in detail hereinabove.

Ex Vivo Expansion of EPC in EPC-positive Conditions: Either purified CD133⁺ cells or cells known to be EPCs were cultured at concentration of 2−100×10³ cells/ml in either 250 ml tissue culture flasks (T-flask 250ml) coated with fibronectin and laminin, or in Teflon tissue culture bags. The medium contained MEMα supplemented with 1 ng/mL carrier-free human recombinant VEGF (R&D), 0.1 μmol/L atorvastatin (Pfizer Inc., NY, N.Y.), and 20% human serum (Baxter Healthcare, Deerfield, Ill.), 2 mM L-glutamine, 25 mM HEPES, 100 μL antibiotics (pen/strep), 1 mM 2-mercaptoethanol and containing the following human recombinant growth/differentiation factors: bFGF, FGF-1 and FGF-2 (each at 40 ng/ml), EGF interleukin-6 (IL-6), OSM and stem cell factor (SCF), each at a final concentration of 10-50 ng/ml with 2-15 μM tetraethylenepentamine (TEPA) (Aldrich, Milwaukee, Wis., USA) and incubated at 37° C. in a humidified atmosphere of 5% CO₂ in air. The cultures were topped up weekly with the same volume of fresh medium, TEPA and growth factors up to thirteen weeks of expansion. For the bioreactor experiments the EPC were immobilized on microcarrier beads (made of Dextran, PGA, Fibrin or Calcium Alginate), as described in detail hereinabove.

### Assessing the Potential and Phenotype of Cells

Self-Renewal Potential Evaluations: The self-renewal potential of stem cells was determined in vitro by long-term colony formation. Cells were washed and seeded in a semi-solid methylcellulose medium supplemented with 2 IU/ml erythropoietin (Eprex, Cilage AG Int., Switzerland), stem cell factor and IL-3, both at 20 ng/ml (Perpo Tech, Inc., Rocky Hill, N.J., USA), G-CSF and GM-CSF, both at 10 ng/ml (Perpo Tech, Inc., Rocky Hill, N.J., USA). The resulting colonies were scored after two weeks of incubation at 37° C. in a humidified atmosphere of 5% CO₂ in air. Colonies were classified as blast, mixed, erythroid, myeloid, and megakaryocytic, according to their cellular composition.

Morphological Assessment: In order to characterize the resulting culture populations, aliquots of cells were deposited on a glass slide (cytocentrifuge, Shandon, Runcom, UK), fixed and stained in May-Grunwald and Giemsa stain.

Surface Antigen Analysis: At different time intervals, the cultured cells were harvested, washed with a PBS solution containing 1% BSA and 0.1% sodium azide (Sigma-Aldrich, St Louis, Mo.), and stained, at 4° C. for 60 minutes, with FITC-labeled anti CD₄₅ monoclonal antibody and either PE-labeled anti CD₃₄ (HPCA-2) monoclonal or PE-labeled control mouse Ig (all from Immunoquality Products, the Netherlands). The cells were then washed with the same PBS solution and were analyzed by a flow cytometer, as described hereinafter.

Flow Cytometry Analysis: Cells were analyzed and sorted using FACS-calibur flow cytometer (Becton-Dickinson, Immunofluorometry systems, Mountain View, Calif.). Cells were passed at a rate of 1,000 cells/second through a 70 μm nozzle, using a saline sheath fluid. A 488 nm argon laser beam at 250 mW served as the light source for excitation. Fluorescence emission of ten thousand cells was measured using a logarithmic amplification and analyzed using CellQuest software.

Calculations: Ex vivo expansion of TNC, CD133, CD133+CD34− cells and CFUc are reported either as cumulative numbers; number of cells per ml multiply by the final culture volume, or as fold-expansion; cumulative numbers divided by initial seeding cell number. CFUc frequency is calculated as number of colonies divided by cell number.

Statistics: The following statistical tests were used: The non-parametric test (Wilcoxon Rank Test) was applied for testing differences between the study groups for quantitative parameters. All tests applied were two-tailed, and p value of 5% or less was considered statistically significant. The data was analyzed using the SAS software (SAS Institute).

### Bioreactors

Static Bioreactors-Teflon Culture Bags: VueLife® FEP Teflon bags (American Fluoroseal Corporation, Gaithersburg, Md.) were used, in volumes of 72 or 290 ml. For growth in the Teflon bag, cells are incubated at 37° C. in a humidified atmosphere of 5% CO₂ in air.

Spinner Flask Bioreactors: Perfusion bioreactors such as the Magna-Flex® Spinner Flasks (Wheaton Science Products, Millville, N.J.) and the Double Sidearm Celstir® Spinner Flasks (Wheaton Science Products, Millville, N.J.) were used as flask-type bioreactors. Spinner flask design and function is described in detail hereinabove.

Rotating Wall Vessel-HARV Bioreactor: The High Aspect Rotational Vessel (HARV) bioreactor (Synthecon, Inc. Houston, Tex.) was used as an example of the rotating wall vessel bioreactor. The design and function of the HARV is described in detail hereinabove. The HARV operates in a standard size incubator, so that no external oxygenator source bubbled into the media is required. Reactor vessel sizes are 10 ml and 50 ml, and are disposable and reusable. Medium is perfused into the bioreactor from a 500 ml reservoir.

### Bioreactor Culture System

The culture system consists of a multiplicity of bioreactors connected to the medium source by sterile plastic tubing. The medium is circulated through the bioreactor with the aid of a roller or centrifugal pump (e.g., KOBETM) or a peristaltic pump. Probes to monitor pH, pO₂ and pCO₂ as well as shear stress and temperature are located in line at points immediately before and following the bioreactor(s). Information from these sensors is monitored electronically. In addition, there is a means for obtaining serial samples of the growth medium in order to monitor glucose, electrolytes, cytokines and growth factors and nutrient concentrations. Activities of cytokines and growth factors are measured by conventional bioassays (e.g., colony forming assays or dependent cell line growth assays) or conventional immunoassays.

### Inoculation with Hematopoietic Stem Cells, MSC or EPCs

A number of HSCs/MSCs/EPCs appropriate to the size of the bioreactor, at a concentration of about 2×10³−1×10⁶ cells/mL, were mixed with an equal volume of serum containing or serum-free media and injected into the bioreactor. In case of MSCs/EPCs circulation of the growth medium is interrupted for a period of about 1-4 hours in order to permit the cells to attach to the surface of the bioreactor core or capillaries or after mounting the cells on microcarriers. No attachment occurs with the HSC, and this step is omitted. Thereafter, the circulator pump was engaged and the growth medium pumped through the system at an initial rate determined by the size of the reactor; a typical rate is about 24 mL/min. Gas exchange occurred via silicone tubes (surface area=0.5 m²) within a stainless steel shell, or by a conventional membrane oxygenator. Polarographic O₂ and CO₂ probes and autoclavable pH electrodes monitor O₂ and CO₂ tensions and pH continuously, respectively. Flow rates were adjusted so as to maintain an optimal O₂ tension (a partial pressure of at least about 30-50 mm of Hg-low oxygen concentration (hypoxia) was recently found to favor renewal and proliferation of hematopoietic stem cells) and optimal physiological pH (7.30-7.45).

When an appropriate number of cells was obtained, as determined by oxygen utilization of the system, a second bioreactor was connected to the system, and cells fed directly into this second bioreactor. Thereafter, the second bioreactor is flushed with fresh growth medium and maintained for up-to 5 weeks for cultivation of the desired hematopoietic components.

### Scaffolds and Hydrogels

Several types of hydrogels that portray different characteristics such as porosity, cell-hydrogel interactions and degradation properties were used in this study. Roughly, the scaffolds used for ex vivo expansion of cells and for tissue engineering can be divided into two groups: synthetic and natural polymers. An exhaustive description of scaffold materials, production and use is brought hereinabove.

### EXPERIMENTAL RESULTS

### EXAMPLE I:

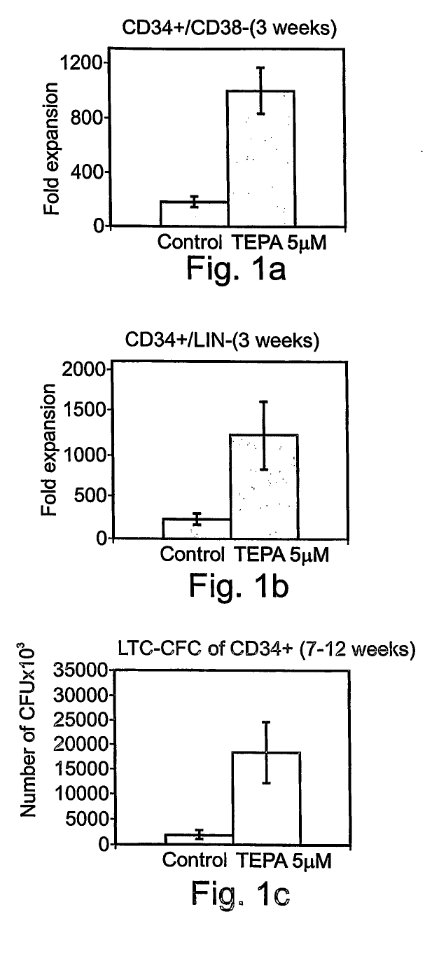

Copper Chelation and Ex Vivo Expansion of HSC in a Gas Permeable Culture Bag: Mononuclear cells (MNC) were collected from either bone marrow (BM), mobilized peripheral blood (MPB) or umbilical cord blood (UCB, as in FIG. 1) and Hematopoietic stem/progenitor cells are isolated by magnetic activated cell sorting (MACS technology, Milteny, Bergisch-Gladbach, GmbH) as described hereinabove. The HSC are then seeded in gas permeable culture bags at concentrations of 1×10⁴ cells/ml in MEM-alpha with 10% Fetal Calf Serum (FCS) containing 50 ng/ml of the following cytokines: SCF, TPO, Flt-3, IL-6 and incubated for at least three weeks in a 5% CO₂ humidified incubator. The culture bags are divided to two groups while the first is supplemented with 5 μM of GC's leading copper chelator tetraethylenepentamine (TEPA, Aldrich, Milwaukee Wis., USA) the other group is not. The culture bags were then replenished once weekly with the same media components. FIG. 1A and B shows the fold expansion of subpopulations of HSC following three weeks of such culture. The two subpopulations CD34⁺/CD38⁻ and CD34⁺/lin⁻ are considered to represent the immature subpopulation of HSC, i.e., the subpopulation that has the major role in self-renewal and proliferation of the HSC. As can be concluded from FIG. 1A and 1B incubation of the cells in the static bioreactor with 5 μM TEPA dramatically increases the fold expansion of these immature subpopulations of hematopoietic stem and/or progenitor cells, indicating the greater long-term potential of HSC cultured in a static bioreactor, according to the methods of the present invention. Furthermore, FIG. 1C shows that in a functional assay, Long Term Culture-Colony Forming Cell (LTC-CFC assay) co-incubation of HSC with 5 μM TEPA increase their numbers dramatically (by at least one order of magnitude) as compared to control cells grown with cytokines, but not with the transition metal chelator (TEPA).

### EXAMPLE II

### Enhanced Ex-Vivo Expansion of Hematopoietic, Mesenchymal and Endothelial Stem Cells Grown With Transition Metal Chelators in Spinner Flask and Rotating Wall Vessel Bioreactors.

As detailed hereinabove, culture in different bioreactor types affords greater opportunity for scaling up of culture volumes, but also requires solution of problems not encountered in simpler, static bioreactors. In order to assess the efficacy of different bioreactor conditions on expansion of stem and/or progenitor cells, HSC, MSC and ESC cultures were expanded in static, spinner flask and rotating wall vessel bioreactors, in the presence of cytokines and transition metal chelator (TEPA).

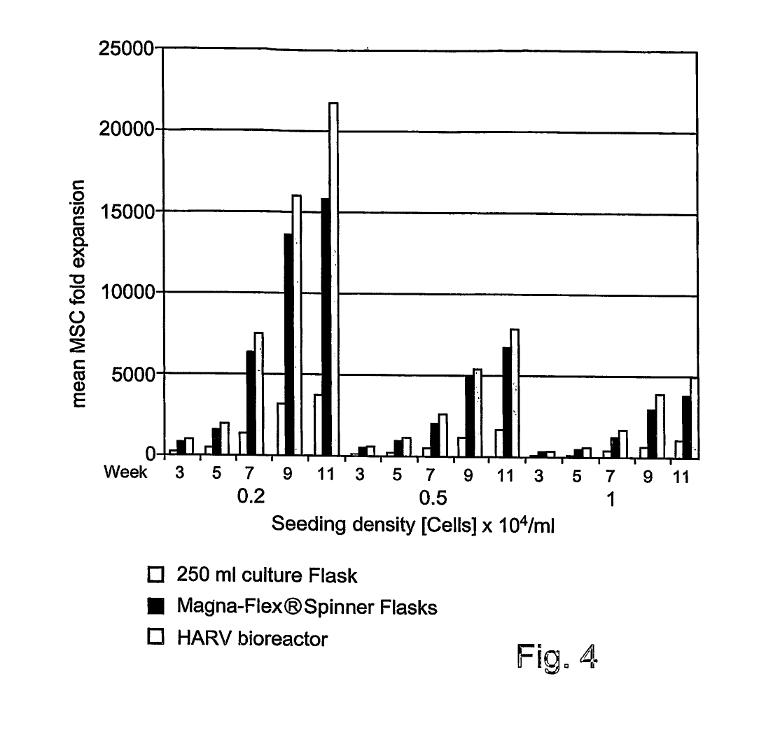

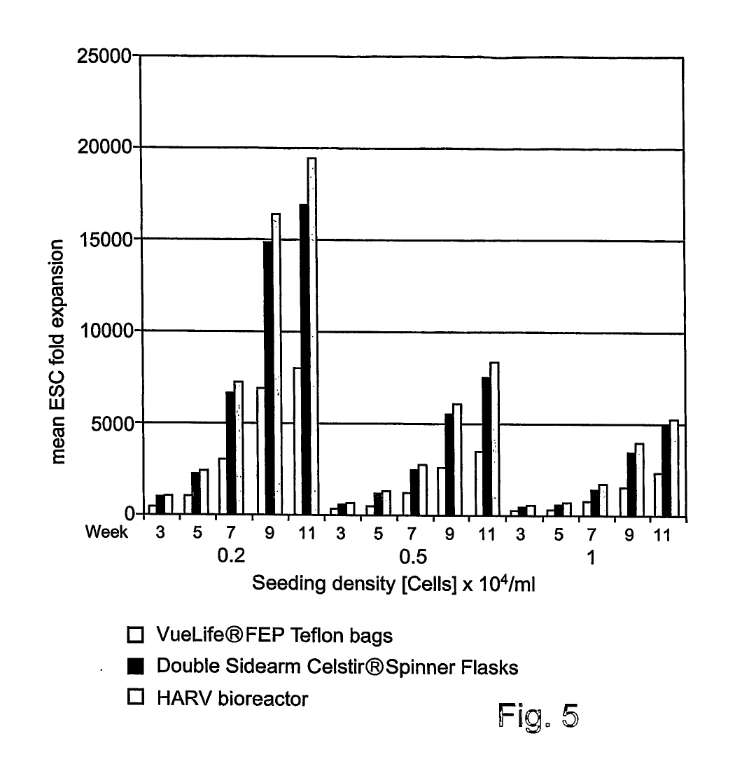

Fold expansion of total nucleated cells cultured with TEPA in the bioreactors, at 3, 5, 7, 9, and 11 weeks of culture, was clearly enhanced by growth conditions in both the spinner flask bioreactor, and the rotating wall vessel bioreactor (HARV) (see FIGS. 3-5), compared with culture in culture bags. Enhanced expansion was observed for cells pre-cultured in conditions favoring HSC development (FIG. 3), MSC development (FIG. 4) and ESC development (FIG. 5). Spinner flask bioreactors produced more efficient expansion of HSC than the HARV bioreactors (FIG. 3), but the HARV bioreactors were more efficient in expanding the MSC and ESC cultures (FIGS. 4 and 5, respectively). In general, lower seeding density (0.2×10⁴ cells/ml) produced the most efficient fold expansion (FIGS. 3-5).

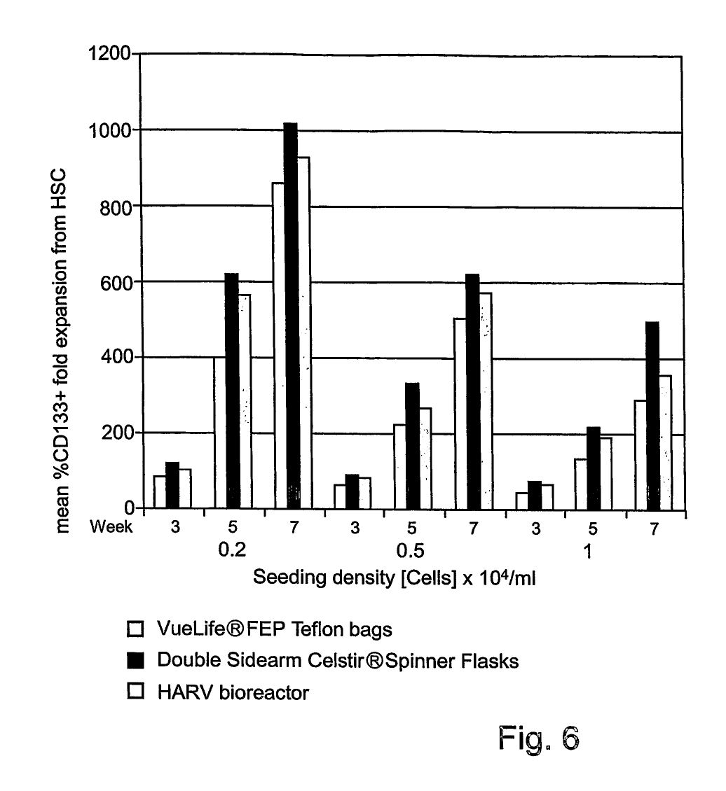

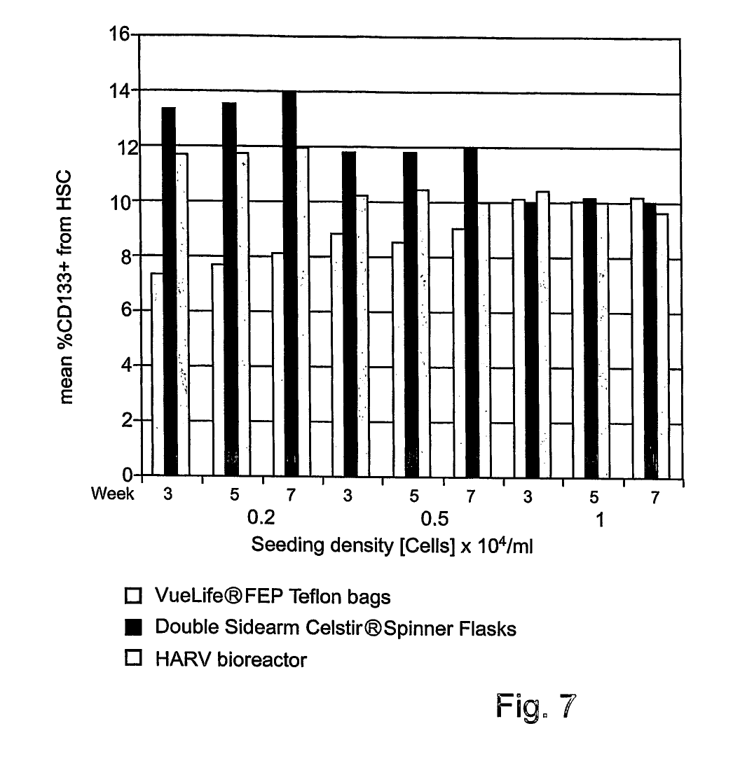

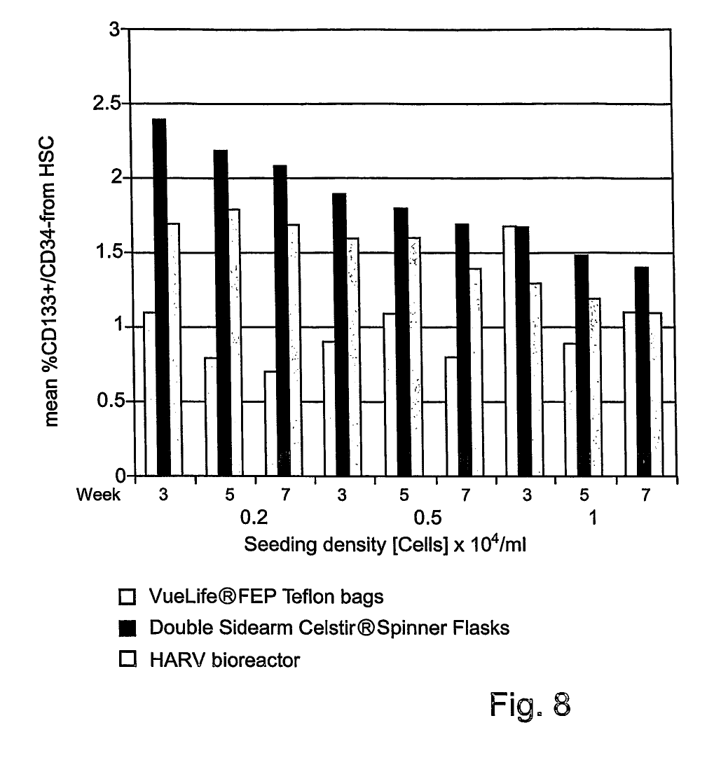

Surprisingly, the bioreactor conditions (including TEPA) were not only favorable for expansion of total nucleated cells, but specifically favorable for expansion of immature and early hematopoietic stem and/or progenitor cells, as indicated by the fold expansion and % of CD133+, and CD133+/CD34− cells detected in the cultures. FIG. 6 shows the mean fold expansion of CD133+ cells from HSC at 3, 5, and 7 weeks culture in the three types of bioreactors. Clearly, culture in the spinner flasks and HARV reactors more efficiently expands the CD133+ fraction, at all seeding densities, compared to culture bags, with a clear advantage for the spinner flask culture. FIGS. 7 and 8, measuring the mean % of CD133+ cells, and CD133+/CD34−0 cells in the bioreactor cultures, respectively, also indicate the strong enhancement of expansion of immature and early hematopoietic stem and/or progenitor cells achieved by culturing in spinner flask or rotating wall vessel (HARV) bioreactors, as compared with static bioreactors (culture bags). Most notably, this enhancement of the mean % of CD133+ and CD133+/CD34− cells is consistent over the 7-week duration of the long term culture in the bioreactors (see FIGS. 7 and 8, 7 weeks), indicating that culture under bioreactor conditions does not impair the self-renewal potential of HSC cultured in the presence of transition metal chelators.

These results show, for the first time, that stem and/or progenitor cells, of diverse lineage, cultured in the presence of cytokines and transition metal chelators in spinner flask and rotating wall bioreactors that afford efficient volume scale-up, show far superior expansion of total nucleated cells, and specifically early stem and/or progenitor cells than cultures grown in static bioreactor (culture bags) conditions. Thus, the use of spinner flasks and rotating wall vessel bioreactors with the culture conditions of the present invention can provide large scale, long term expansion, while inhibiting differentiation, of stem and/or progenitor cells of diverse lineages.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by its accession number was specifically and individually indicated to be incorporated herein by reference. In addition, citation or identification of any reference in this application shall not be construed as an admission that such reference is available as prior art to the present invention.

### REFERENCES CITED

Assady, S., et al. (2001) Diabetes 50 (8): 1691-7.
Ber I, et al. Functional, persistent and extended liver to pancreas transdifferentiation J Biol Chem. 2003
Bonner-Weir, S., et al. (2000). Proc Natl Acad Sci USA 97 (14): 7999-8004.
Brill S, et al. Eur J Cell Biol. 2002 Jan; 81 (1):43-50.
Brill S, et al. Dig Dis Sci. 1999 Feb; 44 (2):364-71.
Deutsch, G et al. (2001) Development 128 (6): 871-81.
Efrat, S. et al. (2001). Curr Opin Investig Drugs 2 (5): 639-42.
Ferber, S., et al. (2000). “Nat Med 6 (5): 568-72.
French, S. W., et al. (2002). Immunol Rev 187: 22-39.
Germain L, et al. Cancer Res. 1988; 48 (17):4909-18.
Hamazaki T, et al. FEBS Lett. 2001; 497 (1):15-9.
Hori, Y., et al. (2002). Proc Natl Acad Sci USA 99 (25): 16105-10.
Jackson, K. A., et al. (2002). J Cell Biochem Suppl 38: 1-6.
Jiang, Y., et al. (2002a). Nature 418 (6893): 41-9.
Jiang, Y., et al. (2002b). Exp Hematol 30 (8): 896-904.
Jones, E. A., et al. (2001). J Anat 198 (Pt 5): 555-9.
Kakinuma S, et al. Stem Cells. 2003; 21 (2):217-27
Kohyama, J., et al. (2001). Differentiation 68 (4-5): 235-44.
Kojima, H., et al. (2003). Nat Med 9 (5): 596-603.
Kojima, H., et al. (2002). Diabetes 51 (5): 1398-408.
Lumelsky, N., et al. (2001). “Science 292 (5520): 1389-94.
Mizuguchi T, et al. J Cell Physiol. 2001; 189 (1):106-19.
Miyazaki M, et al. 2002; 298 (1):24-30.
Odom, D. T., et al. (2004). Science 303 (5662): 1378-81.
Peled, T., et al. (2002). Br. J. Haematol. 116 (3): 655-661.
Petersen B E, et al. Science. 1999; 284 (5417):1168-70.
Petersen B E. Blood Cells Mol Dis. 2001; 27 (3):590-600.
Rambhatla L, et al. Cell Transplant. 2003; 12 (1):1-11.
Ramiya, V. K., et al. (2000). Nat Med 6 (3): 278-82.
Rubinstein, P., et al. (1995). Proc. Natl. Acad. Sci. U.S.A 92 (22): 10119-10122.
Runge D, et al. Biochem Biophys Res Commun. 2000; 269 (1):46-53.
Schwartz R E, et al. J Clin Invest. 2002; 109 (10):1291-302.
Shibata, S., et al. (1997). Lab Anim 31 (2): 163-8.
Soria, B. et al. (2000). Diabetes 49 (2): 157-62.
Soria B, et al. Diabetologia. 2001; 44 (4):407-15.
Strom S C, et al. J Natl Cancer Inst. 1982; 68 (5):771-8.
Suzuki, A., et al. (2002). J Cell Biol 156 (1): 173-84.
Takizawa, Y., et al. (1997). Arch Dermatol Res 289 (4): 213-8.
Tan, C. E. and Moscoso, G. J. (1994). Pathol Int 44 (8): 587-99.
Theise N D, et al. Hepatology. 2000; 31 (1):235-40.
Yamada, T, et al. Stem Cells. 2002; 20 (2):146-54.
Yang, L., et al. (2002). Proc Natl Acad Sci USA 99 (12): 8078-83.
Zalzman, M., et al. (2003). Proc Natl Acad Sci USA.
Abukawa, H., et al. (2003). J Oral Maxillofac Surg 61 (1): 94-100.
Alcorn, M. J., et al. (1996). J Clin Oncol 14 (6): 1839-47.
Altman, G. H., et al. (2002). J Biomech Eng 124 (6): 742-9.
Andrews, R. G., et al. (1994). Curr. Opin. Hematol. 1 (3): 187-196.
Arts, C. H., et al. (2002). Eur J Vasc Endovasc Surg 23 (1): 29-38.
Bachier, C. R., et al. (1999). Exp. Hematol. 27 (4): 615-623.
Bagley, J., et al. (1999). Exp Hematol 27 (3): 496-504.
Banu, N., et al. (2001). Cytokine 13 (6): 349-58.
Barker, J. N. et al. (2002). Curr. Opin. Oncol. 14 (2): 160-164.
Bensinger, W. I., et al. (1996a). Blood 88 (11): 4132-4138.
Bensinger, W. I., et al. (1996b). J. Clin. Oncol. 14 (5): 1447-1456.
Borges, J., et al. (2003). Tissue Eng 9 (3): 441-50.
Briddell, R. A., et al. (1997). J. Hematother. 6 (2): 145-150.
Bruder, S. P., et al. (1998). Clin Orthop (355 Suppl): S247-56.
Brugger, W., et al. (1995). N Engl J Med 333 (5): 283-7.
Carrier, R. L., et al. (1999). Biotechnol Bioeng 64 (5): 580-9.
Cascone, M. G., et al. (2001). J Biomater Sci Polym Ed 12 (3): 267-81.
Cassell, O. C., et al. (2001). Ann NY Acad Sci 944: 429-42.
Chabannon, C., et al. (1999a). Int. J. Oncol. 15 (3): 511-518.
Chabannon, C., et al. (1999b). Hematol. Cell Ther. 41 (2): 78-81.
Chachques, J. C. et al. (2002). J Card Surg 17 (3): 194-200.
Chown, S. R., et al. (1996). Br. J. Haematol. 93 (3): 674-676.
Christensen, R., et al. (2002). 172 (2): 96-104.
Cohen, I., et al. (2000). Biomaterials 21 (21): 2117-23.
Collins, P. C., et al. (1998a). Biotechnol Bioeng 59 (5): 534-43.
Collins, P. C., et al. (1998b). Biotechnol Prog 14 (3): 466-72.
Cotter, T. G., et al. (1994). Immunol Rev 142: 93-112.
Currie, L. J. et al. (2001). Plast Reconstr Surg 108 (6): 1713-26.
Danet, G. H., et al. (2003). J Clin Invest 112 (1): 126-35.
Dar, A., et al. (2002). Biotechnol Bioeng 80 (3): 305-12.
Dybedal, I. and S. E. Jacobsen (1995). Blood 86 (3): 949-57.
Geer, D. J., et al. (2002). Tissue Eng 8 (5): 787-98.
Gehling, U. M., et al. (1997). Exp. Hematol. 25 (11): 1125-1139.
Grassl, E. D., et al. (2002). J Biomed Mater Res 60 (4): 607-12.
Griffith, L. G. and G. Naughton (2002). Science 295 (5557): 1009-14.
Groger, A., et al. (2003). Scand J Plast Reconstr Surg Hand Surg 37 (3): 129-33.
Guerriero, A., L. et al. Blood 1997; 90 (9): 3444-55.
Haisch, A., et al. (2002). Eur Arch Otorhinolaryngol 259 (6): 316-21.
Hildebrand et al. (2001). Ann Biomed Eng 29 (12): 1100-5.
Hoffman, A. S. (2001). Ann NY Acad Sci 944: 62-73.
Hoffman, R., J. et al. (1993). Stem Cells 11 Suppl 2: 76-82.
Hojo, M., et al., Plast Reconstr Surg, 2003, 111 (5): 1638-45.
Huang, Q., et al. Tissue Eng, 2002; 8 (3): 469-82.
Jaroscak, J., et al. Blood, 2003(a).
Jaroscak, J., et al. Blood 2003(b) 101 (12): 5061-7.
Kapelushnik, J., et al. J Pediatr Hematol Oncol, 1998; 20 (3): 257-9.
Karp, J. M., et al. J Craniofac Surg, 2003; 14 (3): 317-23.
Kawamoto, A., et al. Circulation, 2003; 107 (3): 461-8.
Knutsen, G., et al. Tidsskr Nor Laegeforen, 1998; 118 (16): 2493-7.
Kogler, G., et al. Bone Marrow Transplant 1998; 21 Suppl 3: S48-53.
Koller, M. R., et al.,; Biotechnology (NY) 1993(a),11 (3): 358-63.
Koller, M. R., et al. Blood 1993(b); 82 (2): 378-84.
Koller, M. R., et al. Bone Marrow Transplant 1998; 21 (7): 653-63.
Koller, M. R., et al. J Hematother 1995; 4 (3): 159-69.
Kurtzberg, J., et al. N. Engl. J. Med. 1996; 335 (3): 157-166.
LaIuppa, J. A., et al. J Biomed Mater Res, 1997; 36 (3): 347-59.
Laughlin, M. J., et al. N. Engl. J. Med., 2001: 344 (24): 1815-1822.
Leor, J., et al. “Circulation, 2000: 102 (19 Suppl 3): III56-61.
Linenberger, M. L., et al. Exp Hematol 1995;23 (10): 1104-14.
Lisovsky, M., et al. Leukemia, 1996; 10 (6): 1012-8.
Low, H. P., et al. In Vitro Cell Dev Biol Anim. 2001; 37 (3): 141-7.
Mackin, W., et al. Cancer J 2001; 7 Suppl 2: S95-105.
Mandalam, R. K. et al. Mil Med 2002; 167 (2 Suppl): 78-81.
Manosroi, J., et al. Arzneimittelforschung 2002; 52 (1): 60-6.
Mantalaris, A., et al., Biotechnol Prog, 1998; 14 (1): 126-33.
McAdams, T. A., et al. Trends Biotechnol, 1996 (a); 14 (9): 341-9.
McAdams, T. A., et al. Trends Biotechnol 1996 (b); 14 (10): 388-96.
McDowell, C. L. and E. T. Papoutsakis Biotechnol Bioeng 1998; 60 (2): 239-50.
McNiece, I. and R. Briddell Exp. Hematol. 2001; 29 (1): 3-11.
McNiece, I., et al. Blood 2000 (a); 96 (9): 3001-3007.
McNiece, I., et al. Hematol. Cell Ther. 1999; 41 (2): 82-86.
McNiece, I., et al. Exp. Hematol. 2000 (b); 28 (10): 1181-1186.
McNiece, I. K. J. Hematother. Stem Cell Res. 2001; 10 (3): 431-433.
Menasche, P. C R Biol 2002; 325 (6): 731-8.
Murohara, T. Nippon Rinsho 2003; 61 (3): 485-93.
Murray, L. J., et al, Exp Hematol 1999; 27 (6): 1019-28.
Negrin, R. S., et al. Biol. Blood Marrow Transplant. 2000; 6 (3): 262-271.
Neovius, E. B. and G. Kratz Tissue Eng, 2003: 9 (2): 365-9.
Nielsen, L. K. Annu Rev Biomed Eng 1999; 1: 129-52.
Noll, T., , et al. Adv Biochem Eng Biotechnol, 2002; 74: 111-28.
Papadaki, M. IEEE Eng Med Biol Mag 2001; 20 (1): 117, 126.
Patel, S. D., , et al. Biotechnol Prog, 2000; 16 (5): 885-92.
Peled, T., , et al. Br. J. Haematol., 2002; 116 (3): 655-661.
Pereira, R. F., , et al. Proc Natl Acad Sci USA 1998: 95 (3): 1142-7.
Piacibello, W., , et al. Blood, 1997; 89 (8): 2644-53.
Pittenger, M. F., , et al. Science 1999; 284 (5411): 143-7.
Purdy, M. H., , et al. J. Hematother. 1995; 4 (6): 515-525.
Rafii, S. and D. Lyden Nat Med, 2003; 9 (6): 702-12.
Ramsfjell, V., , et al. Blood, 1999; 94 (12): 4093-102.
Richel, D. J., , et al. Bone Marrow Transplant, 2000; 25 (3): 243-249.
Risbud, M. V. and M. Sittinger Trends Biotechnol 2002; 20 (8): 351-6.
Robinson, D., , et al. Cell Transplant 2001; 10(2): 203-8.
Robinson, D., , et al. Clin Orthop 1999; (367 Suppl): S163-75.
Sakai, T., , et al. J Thorac Cardiovasc Surg 1999; 118 (4): 715-24.
Sandstrom, C. E., , et al. Blood 1995; 86 (3): 958-70.
Sandstrom, C. E., , et al. J Hematother 1996; 5 (5): 461-73.
Sardonini, C. A. and Y. J. Wu Biotechnol Prog 1993; 9 (2): 131-7.
Schwartz, R. M., , et al. Proc Natl Acad Sci USA 1991; 88 (15): 6760-4.
Sen, A., , et al. Brain Res Dev Brain Res 2002 (a); 134 (1-2): 103-13.
Sen, A., , et al. Biotechnol Prog 2002 (b) 18 (2): 337-45.
Shapiro, L. and S. Cohen Biomaterials 1997; 18 (8): 583-90.
Shizuru, J. A., , et al. Biol Blood Marrow Transplant 1996; 2 (1): 3-14.
Shpall, E. J., , et al. Blood 2000; 96 (11 (1)): 207a.
Shur, I., , et al., J Cell Biochem 2001; 83 (4): 547-53.
Shur, I., , et al. J Cell Biochem 2002; 87 (1): 51-7.
Sodian, R., , et al. Circulation 2002; 102 (19 Suppl 3): III22-9.
Sodian, R., , et al. Asaio J 2002; 48 (1): 12-6.
Spitzer, R. S., , et al. J Biomed Mater Res 2002; 59 (4): 690-6.
Tock, B., , et al. Haemophilia 1998; 4 (4): 449-55.
Vilquin, J. T., , et al. Arch Mal Coeur Vaiss 2002; 95 (12): 1219-25.
Wagner, J. E., Jr. J. Hematother. 1993; 2 (2): 225-228.
Wagner, J. E., , et al. Blood 1996; 88 (3): 795-802.
Wakitani, S., , et al. Muscle Nerve 1995; 18 (12): 1417-26.
Wechselberger, G., , et al. Plast Reconstr Surg 2002; 110 (1): 123-9.
Williams, S. F., , et al. Int J Biol Macromol 1999; 25 (1-3): 111-21.
Wolff, S. N. Bone Marrow Transplant 2002; 29 (7): 545-52.
Zandstra, P. W., , et al. Biotechnology (NY) 1994; 12 (9): 909-14.

1. A method of ex-vivo expanding stem and/or progenitor cells, while at the same time, substantially inhibiting differentiation of the stem and/or progenitor cells, the method comprising:
(a) obtaining a population of cells comprising stem and/or progenitor cells;
(b) seeding said stem and/or progenitor cells into a bioreactor, and
(c) culturing said stem and/or progenitor cells ex-vivo in said bioreactor under conditions allowing for cell proliferation and, at the same time, culturing said cells under conditions selected from the group consisting of:
(i) conditions reducing expression and/or activity of CD38 in said cells;
(ii) conditions reducing capacity of said cells in responding to signaling pathways involving CD38 in said cells;
(iii) conditions reducing capacity of said cells in responding to retinoic acid, retinoids and/or Vitamin D in said cells;
(iv) conditions reducing capacity of said cells in responding to signaling pathways involving the retinoic acid receptor, the retinoid X receptor and/or the Vitamin D receptor in said cells;
(v) conditions reducing capacity of said cells in responding to signaling pathways involving PI 3-kinase;
(vi) conditions wherein said cells are cultured in the presence of nicotinamide, a nicotinamide analog, a nicotinamide or a nicotinamide analog derivative or a nicotinamide or a nicotinamide analog metabolite;
(vii) conditions wherein said cells are cultured in the presence of a copper chelator;
(viii) conditions wherein said cells are cultured in the presence of a copper chelate;
(ix) conditions wherein said cells are cultured in the presence of a PI 3-kinase inhibitor;
thereby expanding the stem and/or progenitor cells while at the same time, substantially inhibiting differentiation of the stem and/or progenitor cells ex-vivo.

2. The method of claim 1, wherein said stem and/or progenitor cells are derived from a source selected from the group consisting of hematopoietic cells, umbilical cord blood cells, G-CSF mobilized peripheral blood cells, bone marrow cells, hepatic cells, pancreatic cells, intestinal cells, neural cells, oligodendrocyte cells, keratinocytes, skin cells, muscle cells, bone cells, chondrocytes and stroma cells.

3. The method of claim 1, further comprising the step of selecting a population of stem cells enriched for hematopoietic stem cells.

4. The method of claim 3, wherein said selection is affected via CD34.

5. The method of claim 1, further comprising the step of selecting a population of stem cells enriched for early hematopoietic stem/progenitor cells.

6. The method of claim 5, wherein said selection is affected via CD133.

7. The method of claim 1, wherein step (b) is followed by a step comprising selection of stem and/or progenitor cells.

8. The method of claim 7, wherein said selection is affected via CD 133 or CD 34.

9. The method of claim 1, wherein said providing said conditions for cell proliferation is effected by providing the cells with nutrients and cytokines.

10. The method of claim 9, wherein said cytokines are selected from the group consisting of early acting cytokines and late acting cytokines.

11. The method of claim 10, wherein said early acting cytokines are selected from the group consisting of stem cell factor, FLT3 ligand, interleukin-6, thrombopoietin and interleukin-3.

12. The method of claim 10, wherein said late acting cytokines are selected from the group consisting of granulocyte colony stimulating factor, granulocyte/macrophage colony stimulating factor and erythropoietin.

13. The method of claim 10, wherein said late acting cytokine is granulocyte colony stimulating factor.

14. The method of claim 1, wherein said stem and/or progenitor cells are genetically modified cells.

15. The method of claim 1, wherein said inhibitors of PI 3-kinase are wortmannin and/or LY294002.

16. The method of claim 1, wherein said bioreactor is selected from the group consisting of a static bioreactor, a stirred flask bioreactor, a rotating wall vessel bioreactor, a hollow fiber bioreactor and a direct perfusion bioreactor.

17. The method of claim 16, wherein said static bioreactor is selected from the group consisting of well plates, tissue-culture flasks and gas-permeable culture bags.

18. The method of claim 1, wherein said culturing said cells of step (c) is effected in suspension culture.

19. The method of claim 1, wherein said culturing said cells of step (c) is effected on a porous scaffold.

20. The method of claim 19, wherein said porous scaffold is selected from the group consisting of poly (glycolic acid), poly (DL-lactic-co-glycolic acid), alginate, fibronectin, laminin, collagen, hyaluronic acid, Polyhydroxyalkanoate, poly 4 hydroxybutirate (P4HB) and polygluconic acid (PGA).

21. The method of claim 19, wherein said porous scaffold comprises a hydrogel.

22. The method of claim 1, wherein said seeding is static seeding or perfusion seeding.

23. The method of claim 1, wherein said culturing of said cells of steps (b) and (c) is effected without stromal cells or a feeder layer.

24. A conditioned medium isolated from the expanded stem and/or progenitor cell culture of claim 1.

25. A method of preparing a stem and/or progenitor cell conditioned medium, the method comprising:
(a) establishing a stem and/or progenitor cells culture in a bioreactor according to claim 1, thereby expanding the stem and/or progenitor cells while at the same time, substantially inhibiting differentiation of the stem and/or progenitor cells ex-vivo; and
(b) when a desired stem and/or progenitor cell density has been achieved, collecting medium from said bioreactor, thereby obtaining the stem and/or progenitor cell conditioned medium.

26. The stem and/or progenitor cell conditioned medium of claim 25.

27. A method of transplanting ex-vivo expanded stem and/or progenitor cells into a recipient, the method comprising:
(a) obtaining a population of cells comprising stem and/or progenitor cells;
(b) seeding said stem and/or progenitor cells into a bioreactor, and
(c) culturing said stem and/or progenitor cells ex-vivo in said bioreactor under conditions allowing for cell proliferation and, at the same time, culturing said cells under conditions selected from the group consisting of:
(i) conditions reducing expression and/or activity of CD38 in said cells;
(ii) conditions reducing capacity of said cells in responding to signaling pathways involving CD38 in said cells;
(iii) conditions reducing capacity of said cells in responding to retinoic acid, retinoids and/or Vitamin D in said cells;
(iv) conditions reducing capacity of said cells in responding to signaling pathways involving the retinoic acid receptor, the retinoid X receptor and/or the Vitamin D receptor in said cells;
(v) conditions reducing capacity of said cells in responding to signaling pathways involving PI 3-kinase;
(vi) conditions wherein said cells are cultured in the presence of nicotinamide, a nicotinamide analog, a nicotinamide or a nicotinamide analog derivative or a nicotinamide or a nicotinamide analog metabolite;
(vii) conditions wherein said cells are cultured in the presence of a copper chelator;
(viii) conditions wherein said cells are cultured in the presence of a copper chelate;
(ix) conditions wherein said cells are cultured in the presence of a PI 3-kinase inhibitor; and
(d) recovering said expanded stem and/or progenitor cells from said bioreactor, and
(e) transplanting into said recipient said ex-vivo expanded stem and/or progenitor cells produced in steps (b)-(d).

28. The method of claim 27, wherein said stem and/or progenitor cells are derived from a source selected from the group consisting of hematopoietic cells, umbilical cord blood cells, G-CSF mobilized peripheral blood cells, bone marrow cells, hepatic cells, pancreatic cells, intestinal cells, neural cells, oligodendrocyte cells, skin cells, keratinocytes, muscle cells, bone cells, chondrocytes and stroma cells.

29. The method of claim 27, further comprising the step of selecting a population of stem cells enriched for hematopoietic stem cells.

30. The method of claim 29, wherein said selection is affected via CD34.

31. The method of claim 27, further comprising the step of selecting a population of stem cells enriched for early hematopoietic stem/progenitor cells.

32. The method of claim 31, wherein said selection is affected via CD133.

33. The method of claim 27, wherein step (c) is followed by a step comprising selection of stem and/or progenitor cells.

34. The method of claim 33, wherein said selection is affected via CD 133 or CD 34.

35. The method of claim 27, wherein said stem and/or progenitor cells of step (b) are obtained from said recipient.

36. The method of claim 27, wherein said providing said conditions for cell proliferation is effected by providing the cells with nutrients and cytokines.

37. The method of claim 36, wherein said cytokines are selected from the group consisting of early acting cytokines and late acting cytokines.

38. The method of claim 37, wherein said early acting cytokines are selected from the group consisting of stem cell factor, FLT3 ligand, interleukin-6, thrombopoietin and interleukin-3.

39. The method of claim 37, wherein said late acting cytokines are selected from the group consisting of granulocyte colony stimulating factor, granulocyte/macrophage colony stimulating factor and erythropoietin.

40. The method of claim 39, wherein said late acting cytokine is granulocyte colony stimulating factor.

41. The method of claim 27, wherein said stem and/or progenitor cells are genetically modified cells.

42. The method of claim 27, wherein said inhibitors of PI 3-kinase are wortmannin and/or LY294002.

43. The method of claim 27, wherein said bioreactor is selected from the group consisting of a static bioreactor, a stirred flask bioreactor, a rotating wall vessel bioreactor, a hollow fiber bioreactor and a direct perfusion bioreactor.

44. The method of claim 43, wherein said static bioreactor is selected from the group consisting of well plates, tissue-culture flasks and gas-permeable culture bags.

45. The method of claim 27, wherein said culturing said cells of step (c) is effected in suspension culture.

46. The method of claim 27, wherein said culturing said cells of step (c) is effected on a porous scaffold.

47. The method of claim 46, wherein said porous scaffold is selected from the group consisting of poly (glycolic acid), poly (DL-lactic-co-glycolic acid), alginate, fibronectin, laminin, collagen, hyaluronic acid, Polyhydroxyalkanoate, poly 4 hydroxybutirate (P4HB) and polygluconic acid (PGA).

48. The method of claim 41, wherein said porous scaffold comprises a hydrogel.

49. The method of claim 27, wherein said seeding is static seeding or perfusion seeding.

50. The method of claim 27, wherein said culturing of said cells of steps (b) and (c) is effected without stromal cells or a feeder layer.
